SEQUENCE LISTING (147 sequences)

SEQ ID NO: 1 - protein, 11 aa, Artificial Sequence
  Peptide Linker
SGGGGTGGGGS

SEQ ID NO: 2 - protein, 11 aa, Artificial Sequence
  Peptide Linker
SGGGGSGGGGS

SEQ ID NO: 3 - protein, 8 aa, Artificial Sequence
  Hinge linker
DKTHTCAA

SEQ ID NO: 4 - protein, 11 aa, Artificial Sequence
  Hinge linker
DKTHTCPPCPA

SEQ ID NO: 5 - protein, 18 aa, Artificial Sequence
  Hinge linker
DKTHTCPPCPATCPPCPA

SEQ ID NO: 6 - protein, 25 aa, Artificial Sequence
  Hinge linker
DKTHTCPPCPATCPPCPATCPPCPA

SEQ ID NO: 7 - protein, 30 aa, Artificial Sequence
  Hinge linker
DKTHTCPPCPAGKPTLYNSLVMSDTAGTCY

SEQ ID NO: 8 - protein, 31 aa, Artificial Sequence
  Hinge linker
DKTHTCPPCPAGKPTHVNVSVVMAEVDGTCY

SEQ ID NO: 9 - protein, 15 aa, Artificial Sequence
  Hinge linker
DKTHTCCVECPPCPA

SEQ ID NO: 10 - protein, 26 aa, Artificial Sequence
  Hinge linker
DKTHTCPRCPEPKSCDTPPPCPRCPA

SEQ ID NO: 11 - protein, 11 aa, Artificial Sequence
  Hinge linker
DKTHTCPSCPA

SEQ ID NO: 12 - protein, 7 aa, Artificial Sequence
  Flexible linker
SGGGGSE

SEQ ID NO: 13 - protein, 6 aa, Artificial Sequence
  Flexible linker
DKTHTS

SEQ ID NO: 14 - protein, 6 aa, Artificial Sequence
  Flexible linker
SGGGGS

SEQ ID NO: 15 - protein, 11 aa, Artificial Sequence
  Flexible linker
SGGGGSGGGGS

SEQ ID NO: 16 - protein, 16 aa, Artificial Sequence
  Flexible linker
SGGGGSGGGGSGGGGS

SEQ ID NO: 17 - protein, 21 aa, Artificial Sequence
  Flexible linker
SGGGGSGGGGSGGGGSGGGGS

SEQ ID NO: 18 - protein, 26 aa, Artificial Sequence
  Flexible linker
SGGGGSGGGGSGGGGSGGGGSGGGGS

SEQ ID NO: 19 - protein, 11 aa, Artificial Sequence
  Flexible linker
AAAGSGGASAS

SEQ ID NO: 20 - protein, 16 aa, Artificial Sequence
  Flexible linker
  misc_feature (7)..(7): Xaa can be naturally occuring amino acid
AAAGSGXGGGSGASAS

SEQ ID NO: 21 - protein, 21 aa, Artificial Sequence
  Flexible linker
  misc_feature (7)..(7): Xaa can be naturally occuring amino acid
  misc_feature (12)..(12): Xaa can be naturally occuring amino acid
AAAGSGXGGGSXGGGSGASAS

SEQ ID NO: 22 - protein, 26 aa, Artificial Sequence
  Flexible linker
  misc_feature (7)..(7): Xaa can be naturally occuring amino acid
  misc_feature (12)..(12): Xaa can be naturally occuring amino acid
  misc_feature (17)..(17): Xaa can be naturally occuring amino acid
AAAGSGXGGGSXGGGSXGGGSGASAS

SEQ ID NO: 23 - protein, 31 aa, Artificial Sequence
  Flexible linker
  misc_feature (7)..(7): Xaa can be naturally occuring amino acid
  misc_feature (12)..(12): Xaa can be naturally occuring amino acid
  misc_feature (17)..(17): Xaa can be naturally occuring amino acid
  misc_feature (22)..(22): Xaa can be naturally occuring amino acid
AAAGSGXGGGSXGGGSXGGGSXGGGSGASAS

SEQ ID NO: 24 - protein, 13 aa, Artificial Sequence
  Flexible linker
  misc_feature (7)..(7): Xaa can be naturally occuring amino acid
AAAGSGXSGASAS

SEQ ID NO: 25 - protein, 28 aa, Artificial Sequence
  Flexible linker
PGGNRGTTTTRRPATTTGSSPGPTQSHY

SEQ ID NO: 26 - protein, 11 aa, Artificial Sequence
  Flexible linker
ATTTGSSPGPT

SEQ ID NO: 27 - protein, 6 aa, Artificial Sequence
  Flexible linker
ATTTGS

SEQ ID NO: 28 - protein, 21 aa, Artificial Sequence
  Flexible linker
EPSGPISTINSPPSKESHKSP

SEQ ID NO: 29 - protein, 15 aa, Artificial Sequence
  Flexible linker
GTVAAPSVFIFPPSD

SEQ ID NO: 30 - protein, 15 aa, Artificial Sequence
  Flexible linker
GGGGIAPSMVGGGGS

SEQ ID NO: 31 - protein, 15 aa, Artificial Sequence
  Flexible linker
GGGGKVEGAGGGGGS

SEQ ID NO: 32 - protein, 15 aa, Artificial Sequence
  Flexible linker
GGGGSMKSHDGGGGS

SEQ ID NO: 33 - protein, 15 aa, Artificial Sequence
  Flexible linker
GGGGNLITIVGGGGS

SEQ ID NO: 34 - protein, 15 aa, Artificial Sequence
  Flexible linker
GGGGVVPSLPGGGGS

SEQ ID NO: 35 - protein, 12 aa, Artificial Sequence
  Flexible linker
GGEKSIPGGGGS

SEQ ID NO: 36 - protein, 18 aa, Artificial Sequence
  Flexible linker
RPLSYRPPFPFGFPSVRP

SEQ ID NO: 37 - protein, 18 aa, Artificial Sequence
  Flexible linker
YPRSIYIRRRHPSPSLTT

SEQ ID NO: 38 - protein, 18 aa, Artificial Sequence
  Flexible linker
TPSHLSHILPSFGLPTFN

SEQ ID NO: 39 - protein, 18 aa, Artificial Sequence
  Flexible linker
RPVSPFTFPRLSNSWLPA

SEQ ID NO: 40 - protein, 18 aa, Artificial Sequence
  Flexible linker
SPAAHFPRSIPRPGPIRT

SEQ ID NO: 41 - protein, 18 aa, Artificial Sequence
  Flexible linker
APGPSAPSHRSLPSRAFG

SEQ ID NO: 42 - protein, 18 aa, Artificial Sequence
  Flexible linker
PRNSIHFLHPLLVAPLGA

SEQ ID NO: 43 - protein, 18 aa, Artificial Sequence
  Flexible linker
MPSLSGVLQVRYLSPPDL

SEQ ID NO: 44 - protein, 18 aa, Artificial Sequence
  Flexible linker
SPQYPSPLTLTLPPHPSL

SEQ ID NO: 45 - protein, 18 aa, Artificial Sequence
  Flexible linker
NPSLNPPSYLHRAPSRIS

SEQ ID NO: 46 - protein, 17 aa, Artificial Sequence
  Flexible linker
LPWRTSLLPSLPLRRRP

SEQ ID NO: 47 - protein, 18 aa, Artificial Sequence
  Flexible linker
PPLFAKGPVGLLSRSFPP

SEQ ID NO: 48 - protein, 18 aa, Artificial Sequence
  Flexible linker
VPPAPVVSLRSAHARPPY

SEQ ID NO: 49 - protein, 17 aa, Artificial Sequence
  Flexible linker
LRPTPPRVRSYTCCPTP

SEQ ID NO: 50 - protein, 18 aa, Artificial Sequence
  Flexible linker
PNVAHVLPLLTVPWDNLR

SEQ ID NO: 51 - protein, 18 aa, Artificial Sequence
  Flexible linker
CNPLLPLCARSPAVRTFP

SEQ ID NO: 52 - protein, 11 aa, Artificial Sequence
  rigid linker
GAPAPAAPAPA

SEQ ID NO: 53 - protein, 4 aa, Artificial Sequence
  rigid linker
PPPP

SEQ ID NO: 54 - protein, 11 aa, Artificial Sequence
  albumin binding peptide
DLCLRDWGCLW

SEQ ID NO: 55 - protein, 11 aa, Artificial Sequence
  albumin binding peptide
DICLPRWGCLW

SEQ ID NO: 56 - protein, 15 aa, Artificial Sequence
  albumin binding peptide
MEDICLPRWGCLWGD

SEQ ID NO: 57 - protein, 20 aa, Artificial Sequence
  albumin binding peptide
QRLMEDICLPRWGCLWEDDE

SEQ ID NO: 58 - protein, 20 aa, Artificial Sequence
  albumin binding peptide
QGLIGDICLPRWGCLWGRSV

SEQ ID NO: 59 - protein, 21 aa, Artificial Sequence
  albumin binding peptide
QGLIGDICLPRWGCLWGRSVK

SEQ ID NO: 60 - protein, 15 aa, Artificial Sequence
  albumin binding peptide
EDICLPRWGCLWEDD

SEQ ID NO: 61 - protein, 18 aa, Artificial Sequence
  albumin binding peptide
RLMEDICLPRWGCLWEDD

SEQ ID NO: 62 - protein, 16 aa, Artificial Sequence
  albumin binding peptide
MEDICLPRWGCLWEDD

SEQ ID NO: 63 - protein, 15 aa, Artificial Sequence
  albumin binding peptide
MEDICLPRWGCLWED

SEQ ID NO: 64 - protein, 18 aa, Artificial Sequence
  albumin binding peptide
RLMEDICLARWGCLWEDD

SEQ ID NO: 65 - protein, 20 aa, Artificial Sequence
  albumin binding peptide
EVRSFCTRWPAEKSCKPLRG

SEQ ID NO: 66 - protein, 20 aa, Artificial Sequence
  albumin binding peptide
RAPESFVCYWETICFERSEQ

SEQ ID NO: 67 - protein, 11 aa, Artificial Sequence
  albumin binding peptide
EMCYFPGICWM

SEQ ID NO: 68 - protein, 20 aa, Artificial Sequence
  albumin binding peptide
GGGGSGGGGSGGGGSGGGGS

SEQ ID NO: 69 - protein, 16 aa, Artificial Sequence
  flexible linker
SGGGGSGGGGSGGGGS

SEQ ID NO: 70 - protein, 16 aa, Artificial Sequence
  flexible linker
SGGGGSGGGGTGGGGS

SEQ ID NO: 71 - protein, 10 aa, Artificial Sequence
  CDR sequences of antibody 496g3 - CDRH1
GFTFSDYNMA

SEQ ID NO: 72 - protein, 17 aa, Artificial Sequence
  CDR sequences of antibody 496g3 - CDRH2
TITYEGRNTYYRDSVKG

SEQ ID NO: 73 - protein, 16 aa, Artificial Sequence
  CDR sequences of antibody 496g3 - CDRH3
PPQYYEGSIYRLWFAH

SEQ ID NO: 74 - protein, 11 aa, Artificial Sequence
  CDR sequences of antibody 496g3 - CDRL1
RADESVRTLMH

SEQ ID NO: 75 - protein, 7 aa, Artificial Sequence
  CDR sequences of antibody 496g3 - CDRL2
LVSNSEI

SEQ ID NO: 76 - protein, 9 aa, Artificial Sequence
  CDR sequences of antibody 496g3 - CDRL3
QQTWSDPWT

SEQ ID NO: 77 - protein, 107 aa, Artificial Sequence
  Light Chain variable region of antibody 496g3
AIQLTQSPSSLSASVGDRVTITCRADESVRTLMHWYQQKPGKAPKLLIYLVSNSEIGVPDRFSGSGSGTDFRLTISSLQPEDFATYYCQQTWSDPWTFGQGTKVEIK

SEQ ID NO: 78 - protein, 125 aa, Artificial Sequence
  Heavy Chain variable region of antibody 496g3
EVQLVESGGGLVQPGGSLRLSCAASGFTFSDYNMAWVRQAPGKGLEWVATITYEGRNTYYRDSVKGRFTISRDNAKNSLYLQMNSLRAEDTAVYYCASPPQYYEGSIYRLWFAHWGQGTLVTVSS

SEQ ID NO: 79 - DNA, 327 bp, Artificial Sequence
  DNA encoding Light Chain variable region of antibody 496g3
gcgatccagctgacgcaatcgccgtcctcgctgtcagcgagcgtgggcgatagggtcacgatcacctgtcgggccgatgaatcggtccgcactctgatgcattggtatcagcaaaagccgggaaaggccccgaagctgctcatctacctggtgagcaattccgagatcggagtcccggaccgcttcagcggttcgggcagcggaaccgacttccgcctgactatttcctcgctgcaacccgaggacttcgctacttactactgccagcagacctggtcagatccgtggactttcgggcagggaaccaaagtggaaatcaagcggact

SEQ ID NO: 80 - DNA, 375 bp, Artificial Sequence
  DNA encoding Heavy Chain variable region of antibody 496g3
gaagtgcagctggtggaatccggcggaggactggtgcagcccggtggatcattgagactttcgtgtgcagcatcaggctttactttctcggactacaatatggcatgggtgcgccaagcgcctggtaaaggactggaatgggtcgcgactatcacttatgaggggcggaatacttactatagggatagcgtcaaaggacgctttaccatctcacgggacaacgctaagaactccctgtacctccaaatgaactcactcagggcagaagatacggccgtgtactactgtgcctcgcctccgcagtactacgaagggtccatctaccgcctttggttcgcccattggggacagggaacgctggtgactgtgtcctcg

SEQ ID NO: 81 - protein, 214 aa, Artificial Sequence
  Light chain (VL-CL) of antibody 496g3
AIQLTQSPSSLSASVGDRVTITCRADESVRTLMHWYQQKPGKAPKLLIYLVSNSEIGVPDRFSGSGSGTDFRLTISSLQPEDFATYYCQQTWSDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 228 aa, Artificial Sequence
  Heavy chain (VH-CH1) of antibody 496g3
EVQLVESGGGLVQPGGSLRLSCAASGFTFSDYNMAWVRQAPGKGLEWVATITYEGRNTYYRDSVKGRFTISRDNAKNSLYLQMNSLRAEDTAVYYCASPPQYYEGSIYRLWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 83 - DNA, 642 bp, Artificial Sequence
  DNA encoding light chain of antibody 496g3 (no signal sequence)
gcgatccagctgacgcaatcgccgtcctcgctgtcagcgagcgtgggcgatagggtcacgatcacctgtcgggccgatgaatcggtccgcactctgatgcattggtatcagcaaaagccgggaaaggccccgaagctgctcatctacctggtgagcaattccgagatcggagtcccggaccgcttcagcggttcgggcagcggaaccgacttccgcctgactatttcctcgctgcaacccgaggacttcgctacttactactgccagcagacctggtcagatccgtggactttcgggcagggaaccaaagtggaaatcaagcggactgtcgccgcaccttcggtgttcatctttccaccgtcagacgaacaacttaaaagcggaactgcgtcggtggtgtgcctccttaacaacttttaccctagagaagccaaggtccagtggaaggtggacaatgcccttcaaagcggaaacagccaggagtccgtgaccgagcaggactcaaaagattcgacttatagcttgtcgtccacgctcaccctgagcaaagcagactacgaaaagcacaaggtgtacgcttgcgaagtgacccaccaaggcttgtcgagccccgtgaccaaatccttcaaccggggagaatgc

SEQ ID NO: 84 - DNA, 684 bp, Artificial Sequence
  DNA encoding heavy chain of antibody 496g3 (no signal sequence)
gaagtgcagctggtggaatccggcggaggactggtgcagcccggtggatcattgagactttcgtgtgcagcatcaggctttactttctcggactacaatatggcatgggtgcgccaagcgcctggtaaaggactggaatgggtcgcgactatcacttatgaggggcggaatacttactatagggatagcgtcaaaggacgctttaccatctcacgggacaacgctaagaactccctgtacctccaaatgaactcactcagggcagaagatacggccgtgtactactgtgcctcgcctccgcagtactacgaagggtccatctaccgcctttggttcgcccattggggacagggaacgctggtgactgtgtcctcggcttcgaccaaggggccctcggtgttccctctggcgcctagctccaagagcacttcaggcggaacggctgccctcggatgcctggtcaaggactacttcccagagcccgtgaccgtgtcatggaacagcggagctctgactagcggagtgcacacctttccggcggtgctgcaaagctcaggcctgtactcgctctcctcagtggtcactgtcccgtcctcctcgctggggactcaaacgtacatctgcaacgtcaatcacaaaccgtcaaataccaaagtcgacaagaaggtcgagcctaagtcgtgc

SEQ ID NO: 85 - protein, 10 aa, Artificial Sequence
  CDR sequences of antibody 2109 CDRH1
GYTFTDNYIH

SEQ ID NO: 86 - protein, 17 aa, Artificial Sequence
  CDR sequences of antibody 2109 CDRH2
YINPSSAYAHYNEKFKT

SEQ ID NO: 87 - protein, 10 aa, Artificial Sequence
  CDR sequences of antibody 2109 CDRH3
RYYSAMPFAY

SEQ ID NO: 88 - protein, 11 aa, Artificial Sequence
  CDR sequences of antibody 2109 CDRL1
RASEDIYSGLA

SEQ ID NO: 89 - protein, 7 aa, Artificial Sequence
  CDR sequences of antibody 2109 CDRL2
DSSTLHT

SEQ ID NO: 90 - protein, 9 aa, Artificial Sequence
  CDR sequences of antibody 2109 CDRL3
QQNYDFPLT

SEQ ID NO: 91 - protein, 109 aa, Artificial Sequence
  Light Chain variable region of antibody 2109 gL18
DIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGQGTKLEIKRT

SEQ ID NO: 92 - protein, 119 aa, Artificial Sequence
  Heavy Chain variable region of antibody 2109 gH2
EVQLVESGGGLVKPGGSLRLSCAASGYTFTDNYIHWVRQAPGKGLEWIGYINPSSAYAHYNEKFKTRFTISVDKAKNSAYLQMNSLRAEDTAVYYCTRRYYSAMPFAYWGQGTLVTVSS

SEQ ID NO: 93 - DNA, 327 bp, Artificial Sequence
  DNA encoding Light Chain variable region of antibody 2109 gL18
gatatacagatgacccaatcaccaagctctctgagtgcttccgttggcgatcgcgttacaattacctgccgagctagcgaggatatatactcaggactggcctggtaccagcaaaagcctggcaaagtgcctaagctcctgatctacgactccagtaccctgcacactggtgtgccaagccgctttagcggaactggatctggaaccgactatacactgacgatttcctcactgcaaccggaagacgtggcaacctacttctgtcagcaaaactacgacttccccttgacgtttgggcaagggacaaagctggagatcaaacgtacc

SEQ ID NO: 94 - DNA, 357 bp, Artificial Sequence
  DNA encoding Heavy Chain variable region of antibody 2109 gH2
gaagttcaactggtcgaaagcggaggtgggctcgtgaaacctggcggatctctgcgattgtcatgtgctgcaagcggctacacgtttaccgataactatatccactgggtgcgacaagcaccagggaagggactggaatggattggatatattaacccgagctccgcctacgcacactacaacgagaaattcaagacccgattcaccatctccgtggacaaagccaagaactccgcttacctgcaaatgaactctctgcgggccgaagacactgccgtgtattactgcacccgccgatactatagcgctatgccctttgcctactggggacaagggacactggtcactgtctcaagt

SEQ ID NO: 95 - protein, 109 aa, Artificial Sequence
  Light Chain variable region of antibody 2109 gL18
DIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGCGTKLEIKRT

SEQ ID NO: 96 - protein, 119 aa, Artificial Sequence
  Heavy Chain variable region of antibody 2109 gH2
EVQLVESGGGLVKPGGSLRLSCAASGYTFTDNYIHWVRQAPGKCLEWIGYINPSSAYAHYNEKFKTRFTISVDKAKNSAYLQMNSLRAEDTAVYYCTRRYYSAMPFAYWGQGTLVTVSS

SEQ ID NO: 97 - DNA, 327 bp, Artificial Sequence
  DNA encoding Light Chain variable region of antibody 2109 gL18
gatatccagatgacccagtcgccgtccagcctctccgcctccgtgggagacagagtgacgatcacttgcagagcatcagaggacatctactctggccttgcttggtatcagcagaagccgggaaaggtgcccaaactgctcatctatgactcctcgaccctccacacgggagtgccatcgcgcttcagcgggaccggatctgggaccgactacaccctgaccatttcatcgctccagccggaggatgttgccacttacttctgccaacagaattacgacttcccacttacttttggatgtggcactaagctcgaaatcaagcgcacc

SEQ ID NO: 98 - DNA, 357 bp, Artificial Sequence
  DNA encoding Heavy Chain variable region of antibody 2109 gH2
gaagtgcagttggtggagtcggggggagggttggtgaagccaggaggatcattgcggttgtcatgtgcggcttcgggctacactttcactgacaattacattcactgggtgcgacaagcaccagggaagtgcctcgaatggattggctacatcaacccgtcaagcgcatacgcccattacaacgaaaagttcaagacccggttcaccatctccgtggataaggcgaaaaacagcgcgtaccttcagatgaactccctgcgggccgaggataccgccgtttactactgcactagacggtactacagcgccatgccgttcgcgtactggggacaaggcactctggtcaccgtgtcgtcg

SEQ ID NO: 99 - protein, 248 aa, Artificial Sequence
  scFv (VH-VL) of antibody 2109 gH2gL18
EVQLVESGGGLVKPGGSLRLSCAASGYTFTDNYIHWVRQAPGKGLEWIGYINPSSAYAHYNEKFKTRFTISVDKAKNSAYLQMNSLRAEDTAVYYCTRRYYSAMPFAYWGQGTLVTVSSGGGGSGGGGSGGGGSGGGGSDIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGQGTKLEIKRT

SEQ ID NO: 100 - DNA, 744 bp, Artificial Sequence
  DNA encoding scFv (VH-VL) of antibody 2109 gH2gL18
gaagttcaactggtcgaaagcggaggtgggctcgtgaaacctggcggatctctgcgattgtcatgtgctgcaagcggctacacgtttaccgataactatatccactgggtgcgacaagcaccagggaagggactggaatggattggatatattaacccgagctccgcctacgcacactacaacgagaaattcaagacccgattcaccatctccgtggacaaagccaagaactccgcttacctgcaaatgaactctctgcgggccgaagacactgccgtgtattactgcacccgccgatactatagcgctatgccctttgcctactggggacaagggacactggtcactgtctcaagtggaggtggcggttctggcggtggcggttccggtggcggtggatcgggaggtggcggttctgatatacagatgacccaatcaccaagctctctgagtgcttccgttggcgatcgcgttacaattacctgccgagctagcgaggatatatactcaggactggcctggtaccagcaaaagcctggcaaagtgcctaagctcctgatctacgactccagtaccctgcacactggtgtgccaagccgctttagcggaactggatctggaaccgactatacactgacgatttcctcactgcaaccggaagacgtggcaacctacttctgtcagcaaaactacgacttccccttgacgtttgggcaagggacaaagctggagatcaaacgtacc

SEQ ID NO: 101 - protein, 248 aa, Artificial Sequence
  dsscFv (VH-VL) of antibody 2109 gH2gL18
EVQLVESGGGLVKPGGSLRLSCAASGYTFTDNYIHWVRQAPGKCLEWIGYINPSSAYAHYNEKFKTRFTISVDKAKNSAYLQMNSLRAEDTAVYYCTRRYYSAMPFAYWGQGTLVTVSSGGGGSGGGGSGGGGSGGGGSDIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGCGTKLEIKRT

SEQ ID NO: 102 - DNA, 744 bp, Artificial Sequence
  DNA encoding dsscFv (VH-VL) of antibody 2109 gH2gL18
gaagtgcagttggtggagtcggggggagggttggtgaagccaggaggatcattgcggttgtcatgtgcggcttcgggctacactttcactgacaattacattcactgggtgcgacaagcaccagggaagtgcctcgaatggattggctacatcaacccgtcaagcgcatacgcccattacaacgaaaagttcaagacccggttcaccatctccgtggataaggcgaaaaacagcgcgtaccttcagatgaactccctgcgggccgaggataccgccgtttactactgcactagacggtactacagcgccatgccgttcgcgtactggggacaaggcactctggtcaccgtgtcgtcgggaggaggaggctcgggtggaggcggatcgggtggcggagggagcggcggaggcggttcggatatccagatgacccagtcgccgtccagcctctccgcctccgtgggagacagagtgacgatcacttgcagagcatcagaggacatctactctggccttgcttggtatcagcagaagccgggaaaggtgcccaaactgctcatctatgactcctcgaccctccacacgggagtgccatcgcgcttcagcgggaccggatctgggaccgactacaccctgaccatttcatcgctccagccggaggatgttgccacttacttctgccaacagaattacgacttcccacttacttttggatgtggcactaagctcgaaatcaagcgcacc

SEQ ID NO: 103 - protein, 10 aa, Artificial Sequence
  CDR sequences of antibody 645 CDRH1
GIDLSNYAIN

SEQ ID NO: 104 - protein, 16 aa, Artificial Sequence
  CDR sequences of antibody 645 CDRH2
IIWASGTTFYATWAKG

SEQ ID NO: 105 - protein, 13 aa, Artificial Sequence
  CDR sequences of antibody 645 CDRH3
TVPGYSTAPYFDL

SEQ ID NO: 106 - protein, 12 aa, Artificial Sequence
  CDR sequences of antibody 645 CDRL1
QSSPSVWSNFLS

SEQ ID NO: 107 - protein, 7 aa, Artificial Sequence
  CDR sequences of antibody 645 CDRL2
EASKLTS

SEQ ID NO: 108 - protein, 11 aa, Artificial Sequence
  CDR sequences of antibody 645 CDRL3
GGGYSSISDTT

SEQ ID NO: 109 - protein, 112 aa, Artificial Sequence
  Light Chain variable region of antibody 645
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGQGTKVEIKRT

SEQ ID NO: 110 - protein, 121 aa, Artificial Sequence
  Heavy Chain variable region of antibody 645
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 111 - DNA, 336 bp, Artificial Sequence
  DNA encoding Light Chain variable region of antibody 645
gatatccagatgacccagagtccaagcagtgtttccgccagcgtaggcgatcgtgtgactattacctgtcagtcctctccgagcgtttggtccaacttcctgagctggtaccagcagaaaccgggtaaagccccgaaactgctgatctacgaggcgtctaaactgacctctggtgtaccgtcccgtttctctggctctggctctggtacggacttcactctgaccatctcctctctgcagccggaagactttgcaacgtactactgcggtggtggttactcttccatctctgacaccacgttcggtggaggcaccaaagttgaaatcaaacgtacg

SEQ ID NO: 112 - DNA, 363 bp, Artificial Sequence
  DNA encoding Heavy Chain variable region of antibody 645
gaggttcagctgctggagtctggaggcgggcttgtccagcctggagggagcctgcgtctctcttgtgcagtaagcggcatcgacctgtccaactacgcgattaactgggtacgtcaggcaccgggtaaaggtctggaatggatcggcatcatctgggcctctggtacgaccttctacgctacttgggccaaaggtcgtttcaccatctcccgtgacaactctaaaaacaccgtgtacctgcagatgaactctctgcgtgcggaagacactgcggtttactattgcgcgcgtaccgttccgggctattctactgcaccgtacttcgacctgtggggtcagggtactctggttaccgtctcgagt

SEQ ID NO: 113 - protein, 112 aa, Artificial Sequence
  Light Chain variable region of antibody 645
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 114 - protein, 121 aa, Artificial Sequence
  Heavy Chain variable region of antibody 645
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 115 - DNA, 336 bp, Artificial Sequence
  DNA encoding Light Chain variable region of antibody 645
gacatccagatgacgcagtcaccatcgtccgtgtcagcatccgtgggagacagagtgaccattacttgtcagtcctcgccctcagtctggtcgaattttctgtcgtggtatcaacaaaagccagggaaagccccaaagctgctgatctacgaggccagcaaactcacttcgggagtccctagcagattctccggctcgggatcgggcaccgatttcaccctcaccattagctcactccaaccagaggattttgccacctactactgcggcggtggctacagctcaatctcagataccactttcggatgcggtactaaggtcgagattaagcgcact

SEQ ID NO: 116 - DNA, 363 bp, Artificial Sequence
  DNA encoding Heavy Chain variable region of antibody 645
gaagtgcagctcttggaatcgggtggaggactggtgcagccgggaggttccctgaggctgagctgtgctgtgtccggcatcgacctttcaaactacgccatcaattgggtgaggcaggcgccaggaaaatgtctcgaatggatcggtatcatctgggctagcggaactaccttctatgcgacgtgggccaagggacggttcactatctcgcgcgataacagcaagaacaccgtgtacctccagatgaacagcctccgggctgaggacactgcagtctattactgcgcgagaacggtgccgggctactccactgcaccgtacttcgacttgtggggacagggaactcttgtgaccgtcagctcg

SEQ ID NO: 117 - protein, 253 aa, Artificial Sequence
  scFv (VH-VL) of antibody 645
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGQGTKVEIKRT

SEQ ID NO: 118 - DNA, 759 bp, Artificial Sequence
  DNA encoding scFv (VH-VL) of antibody 645
gaggttcagctgctggagtctggaggcgggcttgtccagcctggagggagcctgcgtctctcttgtgcagtaagcggcatcgacctgtccaactacgcgattaactgggtacgtcaggcaccgggtaaaggtctggaatggatcggcatcatctgggcctctggtacgaccttctacgctacttgggccaaaggtcgtttcaccatctcccgtgacaactctaaaaacaccgtgtacctgcagatgaactctctgcgtgcggaagacactgcggtttactattgcgcgcgtaccgttccgggctattctactgcaccgtacttcgacctgtggggtcagggtactctggttaccgtctcgagtggaggtggcggttctggcggtggcggttccggtggcggtggatcgggaggtggcggttctgatatccagatgacccagagtccaagcagtgtttccgccagcgtaggcgatcgtgtgactattacctgtcagtcctctccgagcgtttggtccaacttcctgagctggtaccagcagaaaccgggtaaagccccgaaactgctgatctacgaggcgtctaaactgacctctggtgtaccgtcccgtttctctggctctggctctggtacggacttcactctgaccatctcctctctgcagccggaagactttgcaacgtactactgcggtggtggttactcttccatctctgacaccacgttcggtggaggcaccaaagttgaaatcaaacgtacg

SEQ ID NO: 119 - protein, 253 aa, Artificial Sequence
  dsscFv (VH-VL) of antibody 645
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 120 - DNA, 759 bp, Artificial Sequence
  DNA encoding dsscFv (VH-VL) of antibody 645
gaagtgcagctcttggaatcgggtggaggactggtgcagccgggaggttccctgaggctgagctgtgctgtgtccggcatcgacctttcaaactacgccatcaattgggtgaggcaggcgccaggaaaatgtctcgaatggatcggtatcatctgggctagcggaactaccttctatgcgacgtgggccaagggacggttcactatctcgcgcgataacagcaagaacaccgtgtacctccagatgaacagcctccgggctgaggacactgcagtctattactgcgcgagaacggtgccgggctactccactgcaccgtacttcgacttgtggggacagggaactcttgtgaccgtcagctcgggaggaggaggttcgggcggaggtgggtcgggaggaggtggaagcggaggaggcggatcggacatccagatgacgcagtcaccatcgtccgtgtcagcatccgtgggagacagagtgaccattacttgtcagtcctcgccctcagtctggtcgaattttctgtcgtggtatcaacaaaagccagggaaagccccaaagctgctgatctacgaggccagcaaactcacttcgggagtccctagcagattctccggctcgggatcgggcaccgatttcaccctcaccattagctcactccaaccagaggattttgccacctactactgcggcggtggctacagctcaatctcagataccactttcggatgcggtactaaggtcgagattaagcgcact

SEQ ID NO: 121 - protein, 487 aa, Artificial Sequence
  496.g3 HC-2109VHVL
EVQLVESGGGLVQPGGSLRLSCAASGFTFSDYNMAWVRQAPGKGLEWVATITYEGRNTYYRDSVKGRFTISRDNAKNSLYLQMNSLRAEDTAVYYCASPPQYYEGSIYRLWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGSEVQLVESGGGLVKPGGSLRLSCAASGYTFTDNYIHWVRQAPGKGLEWIGYINPSSAYAHYNEKFKTRFTISVDKAKNSAYLQMNSLRAEDTAVYYCTRRYYSAMPFAYWGQGTLVTVSSGGGGSGGGGSGGGGSGGGGSDIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGQGTKLEIKRT

SEQ ID NO: 122 - DNA, 1461 bp, Artificial Sequence
  DNA encoding 496.g3 HC-2109VHVL
gaagtgcagctggtggaatccggcggaggactggtgcagcccggtggatcattgagactttcgtgtgcagcatcaggctttactttctcggactacaatatggcatgggtgcgccaagcgcctggtaaaggactggaatgggtcgcgactatcacttatgaggggcggaatacttactatagggatagcgtcaaaggacgctttaccatctcacgggacaacgctaagaactccctgtacctccaaatgaactcactcagggcagaagatacggccgtgtactactgtgcctcgcctccgcagtactacgaagggtccatctaccgcctttggttcgcccattggggacagggaacgctggtgactgtgtcctcggcttcgaccaaggggccctcggtgttccctctggcgcctagctccaagagcacttcaggcggaacggctgccctcggatgcctggtcaaggactacttcccagagcccgtgaccgtgtcatggaacagcggagctctgactagcggagtgcacacctttccggcggtgctgcaaagctcaggcctgtactcgctctcctcagtggtcactgtcccgtcctcctcgctggggactcaaacgtacatctgcaacgtcaatcacaaaccgtcaaataccaaagtcgacaagaaggtcgagcctaagtcgtgcagcggaggaggtggatccggcggtggaggtagcgaagttcaactggtcgaaagcggaggtgggctcgtgaaacctggcggatctctgcgattgtcatgtgctgcaagcggctacacgtttaccgataactatatccactgggtgcgacaagcaccagggaagggactggaatggattggatatattaacccgagctccgcctacgcacactacaacgagaaattcaagacccgattcaccatctccgtggacaaagccaagaactccgcttacctgcaaatgaactctctgcgggccgaagacactgccgtgtattactgcacccgccgatactatagcgctatgccctttgcctactggggacaagggacactggtcactgtctcaagtggaggtggcggttctggcggtggcggttccggtggcggtggatcgggaggtggcggttctgatatacagatgacccaatcaccaagctctctgagtgcttccgttggcgatcgcgttacaattacctgccgagctagcgaggatatatactcaggactggcctggtaccagcaaaagcctggcaaagtgcctaagctcctgatctacgactccagtaccctgcacactggtgtgccaagccgctttagcggaactggatctggaaccgactatacactgacgatttcctcactgcaaccggaagacgtggcaacctacttctgtcagcaaaactacgacttccccttgacgtttgggcaagggacaaagctggagatcaaacgtacc

SEQ ID NO: 123 - protein, 487 aa, Artificial Sequence
  496.g3 HC-2109VHVL
EVQLVESGGGLVQPGGSLRLSCAASGFTFSDYNMAWVRQAPGKGLEWVATITYEGRNTYYRDSVKGRFTISRDNAKNSLYLQMNSLRAEDTAVYYCASPPQYYEGSIYRLWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGTGGGGSEVQLVESGGGLVKPGGSLRLSCAASGYTFTDNYIHWVRQAPGKGLEWIGYINPSSAYAHYNEKFKTRFTISVDKAKNSAYLQMNSLRAEDTAVYYCTRRYYSAMPFAYWGQGTLVTVSSGGGGSGGGGSGGGGSGGGGSDIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGQGTKLEIKRT

SEQ ID NO: 124 - DNA, 1461 bp, Artificial Sequence
  DNA encoding 496.g3 HC-2109VHVL
gaagtgcagctggtggaatccggcggaggactggtgcagcccggtggatcattgagactttcgtgtgcagcatcaggctttactttctcggactacaatatggcatgggtgcgccaagcgcctggtaaaggactggaatgggtcgcgactatcacttatgaggggcggaatacttactatagggatagcgtcaaaggacgctttaccatctcacgggacaacgctaagaactccctgtacctccaaatgaactcactcagggcagaagatacggccgtgtactactgtgcctcgcctccgcagtactacgaagggtccatctaccgcctttggttcgcccattggggacagggaacgctggtgactgtgtcctcggcttcgaccaaggggccctcggtgttccctctggcgcctagctccaagagcacttcaggcggaacggctgccctcggatgcctggtcaaggactacttcccagagcccgtgaccgtgtcatggaacagcggagctctgactagcggagtgcacacctttccggcggtgctgcaaagctcaggcctgtactcgctctcctcagtggtcactgtcccgtcctcctcgctggggactcaaacgtacatctgcaacgtcaatcacaaaccgtcaaataccaaagtcgacaagaaggtcgagcctaagtcgtgcagtggaggtgggggcaccggaggtggcggttcagaagttcaactggtcgaaagcggaggtgggctcgtgaaacctggcggatctctgcgattgtcatgtgctgcaagcggctacacgtttaccgataactatatccactgggtgcgacaagcaccagggaagggactggaatggattggatatattaacccgagctccgcctacgcacactacaacgagaaattcaagacccgattcaccatctccgtggacaaagccaagaactccgcttacctgcaaatgaactctctgcgggccgaagacactgccgtgtattactgcacccgccgatactatagcgctatgccctttgcctactggggacaagggacactggtcactgtctcaagtggaggtggcggttctggcggtggcggttccggtggcggtggatcgggaggtggcggttctgatatacagatgacccaatcaccaagctctctgagtgcttccgttggcgatcgcgttacaattacctgccgagctagcgaggatatatactcaggactggcctggtaccagcaaaagcctggcaaagtgcctaagctcctgatctacgactccagtaccctgcacactggtgtgccaagccgctttagcggaactggatctggaaccgactatacactgacgatttcctcactgcaaccggaagacgtggcaacctacttctgtcagcaaaactacgacttccccttgacgtttgggcaagggacaaagctggagatcaaacgtacc

SEQ ID NO: 125 - protein, 487 aa, Artificial Sequence
  496.g3 HC-2109dsVHVL
EVQLVESGGGLVQPGGSLRLSCAASGFTFSDYNMAWVRQAPGKGLEWVATITYEGRNTYYRDSVKGRFTISRDNAKNSLYLQMNSLRAEDTAVYYCASPPQYYEGSIYRLWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGSEVQLVESGGGLVKPGGSLRLSCAASGYTFTDNYIHWVRQAPGKCLEWIGYINPSSAYAHYNEKFKTRFTISVDKAKNSAYLQMNSLRAEDTAVYYCTRRYYSAMPFAYWGQGTLVTVSSGGGGSGGGGSGGGGSGGGGSDIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGCGTKLEIKRT

SEQ ID NO: 126 - DNA, 1461 bp, Artificial Sequence
  DNA encoding 496.g3 HC-2109dsVHVL
gaagtgcagctggtggaatccggcggaggactggtgcagcccggtggatcattgagactttcgtgtgcagcatcaggctttactttctcggactacaatatggcatgggtgcgccaagcgcctggtaaaggactggaatgggtcgcgactatcacttatgaggggcggaatacttactatagggatagcgtcaaaggacgctttaccatctcacgggacaacgctaagaactccctgtacctccaaatgaactcactcagggcagaagatacggccgtgtactactgtgcctcgcctccgcagtactacgaagggtccatctaccgcctttggttcgcccattggggacagggaacgctggtgactgtgtcctcggcttcgaccaaggggccctcggtgttccctctggcgcctagctccaagagcacttcaggcggaacggctgccctcggatgcctggtcaaggactacttcccagagcccgtgaccgtgtcatggaacagcggagctctgactagcggagtgcacacctttccggcggtgctgcaaagctcaggcctgtactcgctctcctcagtggtcactgtcccgtcctcctcgctggggactcaaacgtacatctgcaacgtcaatcacaaaccgtcaaataccaaagtcgacaagaaggtcgagcctaagtcgtgcagcggaggaggtggatccggcggtggaggtagcgaagtgcagttggtggagtcggggggagggttggtgaagccaggaggatcattgcggttgtcatgtgcggcttcgggctacactttcactgacaattacattcactgggtgcgacaagcaccagggaagtgcctcgaatggattggctacatcaacccgtcaagcgcatacgcccattacaacgaaaagttcaagacccggttcaccatctccgtggataaggcgaaaaacagcgcgtaccttcagatgaactccctgcgggccgaggataccgccgtttactactgcactagacggtactacagcgccatgccgttcgcgtactggggacaaggcactctggtcaccgtgtcgtcgggaggaggaggctcgggtggaggcggatcgggtggcggagggagcggcggaggcggttcggatatccagatgacccagtcgccgtccagcctctccgcctccgtgggagacagagtgacgatcacttgcagagcatcagaggacatctactctggccttgcttggtatcagcagaagccgggaaaggtgcccaaactgctcatctatgactcctcgaccctccacacgggagtgccatcgcgcttcagcgggaccggatctgggaccgactacaccctgaccatttcatcgctccagccggaggatgttgccacttacttctgccaacagaattacgacttcccacttacttttggatgtggcactaagctcgaaatcaagcgcacc

SEQ ID NO: 127 - protein, 487 aa, Artificial Sequence
  496.g3 HC-2109dsVHVL
EVQLVESGGGLVQPGGSLRLSCAASGFTFSDYNMAWVRQAPGKGLEWVATITYEGRNTYYRDSVKGRFTISRDNAKNSLYLQMNSLRAEDTAVYYCASPPQYYEGSIYRLWFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGTGGGGSEVQLVESGGGLVKPGGSLRLSCAASGYTFTDNYIHWVRQAPGKCLEWIGYINPSSAYAHYNEKFKTRFTISVDKAKNSAYLQMNSLRAEDTAVYYCTRRYYSAMPFAYWGQGTLVTVSSGGGGSGGGGSGGGGSGGGGSDIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGCGTKLEIKRT

SEQ ID NO: 128 - DNA, 1461 bp, Artificial Sequence
  DNA encoding 496.g3 HC-2109dsVHVL
gaagtgcagctggtggaatccggcggaggactggtgcagcccggtggatcattgagactttcgtgtgcagcatcaggctttactttctcggactacaatatggcatgggtgcgccaagcgcctggtaaaggactggaatgggtcgcgactatcacttatgaggggcggaatacttactatagggatagcgtcaaaggacgctttaccatctcacgggacaacgctaagaactccctgtacctccaaatgaactcactcagggcagaagatacggccgtgtactactgtgcctcgcctccgcagtactacgaagggtccatctaccgcctttggttcgcccattggggacagggaacgctggtgactgtgtcctcggcttcgaccaaggggccctcggtgttccctctggcgcctagctccaagagcacttcaggcggaacggctgccctcggatgcctggtcaaggactacttcccagagcccgtgaccgtgtcatggaacagcggagctctgactagcggagtgcacacctttccggcggtgctgcaaagctcaggcctgtactcgctctcctcagtggtcactgtcccgtcctcctcgctggggactcaaacgtacatctgcaacgtcaatcacaaaccgtcaaataccaaagtcgacaagaaggtcgagcctaagtcgtgcagtggaggtgggggcaccggaggtggcggttcagaagtgcagttggtggagtcggggggagggttggtgaagccaggaggatcattgcggttgtcatgtgcggcttcgggctacactttcactgacaattacattcactgggtgcgacaagcaccagggaagtgcctcgaatggattggctacatcaacccgtcaagcgcatacgcccattacaacgaaaagttcaagacccggttcaccatctccgtggataaggcgaaaaacagcgcgtaccttcagatgaactccctgcgggccgaggataccgccgtttactactgcactagacggtactacagcgccatgccgttcgcgtactggggacaaggcactctggtcaccgtgtcgtcgggaggaggaggctcgggtggaggcggatcgggtggcggagggagcggcggaggcggttcggatatccagatgacccagtcgccgtccagcctctccgcctccgtgggagacagagtgacgatcacttgcagagcatcagaggacatctactctggccttgcttggtatcagcagaagccgggaaaggtgcccaaactgctcatctatgactcctcgaccctccacacgggagtgccatcgcgcttcagcgggaccggatctgggaccgactacaccctgaccatttcatcgctccagccggaggatgttgccacttacttctgccaacagaattacgacttcccacttacttttggatgtggcactaagctcgaaatcaagcgcacc

SEQ ID NO: 129 - protein, 478 aa, Artificial Sequence
  496.g3 LC-645VHVL
AIQLTQSPSSLSASVGDRVTITCRADESVRTLMHWYQQKPGKAPKLLIYLVSNSEIGVPDRFSGSGSGTDFRLTISSLQPEDFATYYCQQTWSDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGQGTKVEIKRT

SEQ ID NO: 130 - DNA, 1434 bp, Artificial Sequence
  DNA encoding 496.g3 LC-645VHVL
gcgatccagctgacgcaatcgccgtcctcgctgtcagcgagcgtgggcgatagggtcacgatcacctgtcgggccgatgaatcggtccgcactctgatgcattggtatcagcaaaagccgggaaaggccccgaagctgctcatctacctggtgagcaattccgagatcggagtcccggaccgcttcagcggttcgggcagcggaaccgacttccgcctgactatttcctcgctgcaacccgaggacttcgctacttactactgccagcagacctggtcagatccgtggactttcgggcagggaaccaaagtggaaatcaagcggactgtcgccgcaccttcggtgttcatctttccaccgtcagacgaacaacttaaaagcggaactgcgtcggtggtgtgcctccttaacaacttttaccctagagaagccaaggtccagtggaaggtggacaatgcccttcaaagcggaaacagccaggagtccgtgaccgagcaggactcaaaagattcgacttatagcttgtcgtccacgctcaccctgagcaaagcagactacgaaaagcacaaggtgtacgcttgcgaagtgacccaccaaggcttgtcgagccccgtgaccaaatccttcaaccggggagaatgcagcggtggcggaggctccggaggaggaggatcagaggttcagctgctggagtctggaggcgggcttgtccagcctggagggagcctgcgtctctcttgtgcagtaagcggcatcgacctgtccaactacgcgattaactgggtacgtcaggcaccgggtaaaggtctggaatggatcggcatcatctgggcctctggtacgaccttctacgctacttgggccaaaggtcgtttcaccatctcccgtgacaactctaaaaacaccgtgtacctgcagatgaactctctgcgtgcggaagacactgcggtttactattgcgcgcgtaccgttccgggctattctactgcaccgtacttcgacctgtggggtcagggtactctggttaccgtctcgagtggaggtggcggttctggcggtggcggttccggtggcggtggatcgggaggtggcggttctgatatccagatgacccagagtccaagcagtgtttccgccagcgtaggcgatcgtgtgactattacctgtcagtcctctccgagcgtttggtccaacttcctgagctggtaccagcagaaaccgggtaaagccccgaaactgctgatctacgaggcgtctaaactgacctctggtgtaccgtcccgtttctctggctctggctctggtacggacttcactctgaccatctcctctctgcagccggaagactttgcaacgtactactgcggtggtggttactcttccatctctgacaccacgttcggtggaggcaccaaagttgaaatcaaacgtacg

SEQ ID NO: 131 - protein, 478 aa, Artificial Sequence
  496.g3 LC-645dsVHVL
AIQLTQSPSSLSASVGDRVTITCRADESVRTLMHWYQQKPGKAPKLLIYLVSNSEIGVPDRFSGSGSGTDFRLTISSLQPEDFATYYCQQTWSDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 132 - DNA, 1434 bp, Artificial Sequence
  DNA encoding 496.g3 LC-645dsVHVL
gcgatccagctgacgcaatcgccgtcctcgctgtcagcgagcgtgggcgatagggtcacgatcacctgtcgggccgatgaatcggtccgcactctgatgcattggtatcagcaaaagccgggaaaggccccgaagctgctcatctacctggtgagcaattccgagatcggagtcccggaccgcttcagcggttcgggcagcggaaccgacttccgcctgactatttcctcgctgcaacccgaggacttcgctacttactactgccagcagacctggtcagatccgtggactttcgggcagggaaccaaagtggaaatcaagcggactgtcgccgcaccttcggtgttcatctttccaccgtcagacgaacaacttaaaagcggaactgcgtcggtggtgtgcctccttaacaacttttaccctagagaagccaaggtccagtggaaggtggacaatgcccttcaaagcggaaacagccaggagtccgtgaccgagcaggactcaaaagattcgacttatagcttgtcgtccacgctcaccctgagcaaagcagactacgaaaagcacaaggtgtacgcttgcgaagtgacccaccaaggcttgtcgagccccgtgaccaaatccttcaaccggggagaatgcagcggtggcggaggctccggaggaggaggatcagaagtgcagctcttggaatcgggtggaggactggtgcagccgggaggttccctgaggctgagctgtgctgtgtccggcatcgacctttcaaactacgccatcaattgggtgaggcaggcgccaggaaaatgtctcgaatggatcggtatcatctgggctagcggaactaccttctatgcgacgtgggccaagggacggttcactatctcgcgcgataacagcaagaacaccgtgtacctccagatgaacagcctccgggctgaggacactgcagtctattactgcgcgagaacggtgccgggctactccactgcaccgtacttcgacttgtggggacagggaactcttgtgaccgtcagctcgggaggaggaggttcgggcggaggtgggtcgggaggaggtggaagcggaggaggcggatcggacatccagatgacgcagtcaccatcgtccgtgtcagcatccgtgggagacagagtgaccattacttgtcagtcctcgccctcagtctggtcgaattttctgtcgtggtatcaacaaaagccagggaaagccccaaagctgctgatctacgaggccagcaaactcacttcgggagtccctagcagattctccggctcgggatcgggcaccgatttcaccctcaccattagctcactccaaccagaggattttgccacctactactgcggcggtggctacagctcaatctcagataccactttcggatgcggtactaaggtcgagattaagcgcact

SEQ ID NO: 133 - protein, 119 aa, Artificial Sequence
  Rat variable heavy chain sequence of antibody 2109
EVQLHQSGAALVKPGASVKLSCKTSGYTFTDNYIHWVKQSPGKSLEWIGYINPSSAYAHYNEKFKTKATLTVDKSTNTAYMELSRLTSEDSATYFCTRRYYSAMPFAYWGQGTLVTVSS

SEQ ID NO: 134 - protein, 113 aa, Artificial Sequence
  human germline acceptor framework VH3 sequence 1-3 3-21 with JH4
EVQLVESGGGLVKPGGSLRLSCAASGFTFSSYSMNWVRQAPGKGLEWVSSISSSTSYIYYADSVKGRFTISRDNAKNSLYLQMNSLRAEDTAVYYCARYFDYWGQGTLVTVSS

SEQ ID NO: 135 - protein, 119 aa, Artificial Sequence
  Heavy Chain variable region of antibody 2109 gH1
EVQLVESGGGLVKPGGSLRLSCATSGYTFTDNYIHWVRQAPGKGLEWIGYINPSSAYAHYNEKFKTRFTISVDKAKNSAYLQMNSLRAEDTAVYYCTRRYYSAMPFAYWGQGTLVTVSS

SEQ ID NO: 136 - protein, 11 aa, Artificial Sequence
  CDR sequences of antibody 2109 CDRL1
RASEDIYNGLA

SEQ ID NO: 137 - protein, 7 aa, Artificial Sequence
  CDR sequences of antibody 2109 CDRL2
NSNTLHT

SEQ ID NO: 138 - protein, 7 aa, Artificial Sequence
  CDR sequences of antibody 2109 CDRL2
NSSTLHT

SEQ ID NO: 139 - protein, 7 aa, Artificial Sequence
  CDR sequences of antibody 2109 CDRL2
DSNTLHT

SEQ ID NO: 140 - protein, 107 aa, Artificial Sequence
  Light Chain variable region of rat antibody 2109
DIVMTQSPASLSASLGETVTIECRASEDIYNGLAWYQQKPGKSPHLLIYNSNTLHTGVPSRFSGTGSGTQYSLKINSLQSEDVATYFCQQNYDFPLTFGSGTKLELK

SEQ ID NO: 141 - protein, 107 aa, Artificial Sequence
  Human VK1 2-1(U) A20 JK2 acceptor framework
DIQMTQSPSSLSASVGDRVTITCRASQGISNYLAWYQQKPGKVPKLLIYAASTLQSGVPSRFSGSGSGTDFTLTISSLQPEDVATYYCQKYNSAPYTFGQGTKLEIK

SEQ ID NO: 142 - protein, 107 aa, Artificial Sequence
  Light Chain variable region of antibody 2109 gL1
DIQMTQSPSSLSASVGDRVTITCRASEDIYNGLAWYQQKPGKVPKLLIYNSNTLHTGVPSRFSGTGSGTDYTLTISSLQPEDVATYFCQQNYDFPLTFGQGTKLEIK

SEQ ID NO: 143 - DNA, 1461 bp, Artificial Sequence
  DNA encoding 496.g3 HC-2109dsVHVL
gaggtgcagctcgtcgaaagcggcggaggactcgtgcagcctggaggctcattgcggttgtcatgtgccgccagcggcttcaccttttccgactataacatggcctgggtgcgccaagctcctggaaaggggctggaatgggtcgccaccatcacttacgagggccggaacacctactaccgcgattccgtgaaggggcggttcactatttcgcgggacaacgccaagaactcgctgtaccttcaaatgaactccctccgcgcggaagataccgccgtgtattactgcgcctcaccaccgcaatactacgagggaagcatctacagactctggttcgcccattggggccagggaactttggtgaccgtcagctcagcctccactaagggccccagcgtgttccctcttgctccgtcctccaagtccacttctggcggcactgcagccttggggtgcctcgtgaaggattacttccccgaacccgtgaccgtgtcctggaactccggagccctgacctcgggagtgcacaccttcccagcggtgctccagtcctccggcctgtactcactctcatcggtcgtgaccgtgccgtccagctccctgggaacccagacttacatttgcaacgtgaaccacaagccatccaacaccaaggtggacaagaaggtggagcctaagagctgttcgggaggaggcggttctggtggcggaggatcggaggtgcagcttgtggagtccggtggaggccttgtgaaacctggcggaagcctgaggctgtcatgtgccgcctccggatacaccttcactgacaactacatccactgggtgcgccaagctccgggaaagtgcctggaatggattggctacattaacccctcctccgcctacgcccactacaacgaaaagttcaagacccggttcaccatctccgtcgacaaggccaagaatagcgcctatctgcaaatgaacagcctccgcgcggaagataccgcggtgtactactgcactcggagatactacagcgccatgccgttcgcgtactggggacagggcaccttggtcaccgtgtcaagcggaggaggaggctccggtggtggaggatcggggggaggaggaagcggagggggtggttcagatattcagatgactcagtccccttcgtccctgagcgcgtcagtgggcgacagggtcaccattacctgtcgggcttccgaggacatctactcaggactcgcctggtatcaacagaagccgggaaaggtcccgaagctcctgatctacgactcgtcgaccctgcacactggcgtgccatcccgattttcgggaacgggctcagggactgactacactctgaccatttcaagcctgcagcctgaggatgtggccacttacttctgccaacaaaactacgacttcccccttaccttcggctgcgggactaagctggagatcaagcggact

SEQ ID NO: 144 - DNA, 1480 bp, Artificial Sequence
  DNA encoding 496.g3 HC-2109dsVHVL
gaagtgcagctggtggaatctggcggcggactggtgcagcctggcggatctctgagactgtcttgtgccgcctccggcttcaccttctccgactacaacatggcctgggtgcgacaggctcctggcaagggactggaatgggtggccacaatcacctacgagggccggaacacctactaccgggactctgtgaagggccggttcaccatctctcgggacaacgccaagaactccctgtacctgcagatgaacagcctgcgggccgaggacaccgccgtgtactactgtgctagcccccctcagtactatgagggctccatctaccggctgtggttcgcccattggggccagggcacactcgtgaccgtgtcctctgcttccaccaagggcccctccgtgtttcctctggccccttccagcaagtccacctctggcggaacagccgctctgggctgcctcgtgaaggactacttccccgagcccgtgacagtgtcttggaactctggcgccctgacctccggcgtgcacacctttccagctgtgctgcagtcctccggcctgtactccctgtcctccgtcgtgactgtgccctccagctctctgggcacccagacctacatctgcaacgtgaaccacaagccctccaacaccaaggtggacaagaaggtggaacccaagtcctgcagcggaggcggtggttcaggcggcggaggatctgaggtgcagctggtggaaagtgggggaggcctcgtgaaaccaggcggcagtctgagactgagctgcgccgcttctggctacacctttaccgacaactacatccactgggtgcgccaggccccagggaaatgcctggaatggatcggctacatcaacccctccagcgcctacgcccactacaacgagaagttcaagacccgcttcaccatcagcgtggacaaggctaagaatagcgcctacctgcagatgaactccctgagagctgaggataccgctgtgtattattgcacccggcggtactactccgccatgccctttgcttactggggacagggaaccctcgtgacagtgtctagcggcggaggcggaagtggtggtggtggatctgggggaggcggttctggtggtggcggctctgatatccagatgacccagtcccccagctccctgtctgcctctgtgggcgacagagtgaccatcacctgtcgggcctccgaggacatctactctggcctggcctggtatcagcagaaacccggcaaggtgcccaagctgctgatctacgactcctccaccctgcacaccggcgtgccctctagattttccggcaccggctctggcaccgactataccctgaccatctccagcctgcagcctgaggacgtggccacctacttttgccagcagaactacgacttccctctgaccttcggctgcggcaccaagctggaaatcaagcggacctgatagctcgaggctatca

SEQ ID NO: 145 - DNA, 1434 bp, Artificial Sequence
  DNA encoding 496.g3 LC-645dsVHVL
gcaatccaactgacccagagcccctcctccctctccgcctccgtgggagacagagtgaccatcacttgccgggccgatgagtccgtgagaactctcatgcactggtatcagcaaaagccagggaaagctccgaagctgctgatctacctggtgtcaaattccgagattggcgtgcctgaccggttcagcgggagcggatcaggcaccgacttcagactcacgatctcatccctgcaaccggaagattttgccacgtactactgccaacagacctggtccgacccctggacattcggtcagggcaccaaggtcgaaatcaagaggactgtggccgctccttccgtgtttatcttccctccgtccgacgaacagctgaaatccggaaccgccagcgtcgtgtgcctgctgaacaatttctacccgcgcgaagccaaggtccagtggaaggtcgacaacgccctccagtccggaaactcccaggagtcggtgaccgaacaggattcgaaggactcgacctactccctgtcctcgactttgaccttgtccaaggccgattacgagaagcataaggtctacgcctgcgaagtgacccatcaggggctgtcatcccccgtgaccaagagcttcaaccgcggggagtgctcgggaggaggaggctcaggaggagggggttccgaagtgcagctgctggaatccggaggaggtctggtgcagccagggggttctctgcggctttcctgcgctgtcagcgggatcgacctctccaactacgccatcaattgggtgcgacaagctccgggaaagtgcctggagtggattggaatcatttgggccagcggcactaccttctacgcgacttgggcgaagggtcgcttcaccatctcccgggacaattcgaagaacaccgtgtacttgcagatgaacagcctgagagccgaggatacggccgtgtactactgtgcgaggactgtgcctggctactcgaccgccccctacttcgatctttggggacagggcacactggtcaccgtgtcatccggtggaggaggatcggggggaggaggttccggaggcggtggtagcggaggcggaggttccgacatccaaatgacccagtccccgtcgagcgtgtccgcctcagtcggggacagagtgaccatcacatgccagtcctcgccctcggtctggtccaacttcctgtcctggtatcagcagaaacccggaaaggcccccaagctgctcatctacgaagcctccaagctgaccagcggggtgccttcgaggttctctggctcgggatcaggcaccgactttaccctcacgatttcgagcctgcaaccggaggatttcgcaacttactactgcggcggtggttacagctccattagcgacaccaccttcggctgcgggaccaaggtcgagatcaagcgcact

SEQ ID NO: 146 - DNA, 1447 bp, Artificial Sequence
  DNA encoding 496.g3 LC-645dsVHVL
gccatccagctgacccagagcccttccagcctgtctgcctctgtgggcgacagagtgaccatcacctgtcgggccgatgagtccgtgcggaccctgatgcactggtatcagcagaagcccggcaaggcccccaagctgctgatctacctggtgtccaactccgagatcggcgtgcccgacagattctccggctctggctctggcaccgacttccggctgaccatctctagcctgcagcccgaggacttcgccacctactactgccagcagacttggagcgacccctggacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgttctggcggcggaggatctgggggaggcggatctgaagtgcagctgctggaatctggcggaggcctggtgcagcctggcggatctctgagactgtcctgtgccgtgtccggcatcgacctgtccaactacgccatcaactgggtgcgacaggcccctggcaagtgcctggaatggatcggcatcatctgggcctccggcaccaccttctacgccacctgggctaagggccggttcaccatctcccgggacaactccaagaacaccgtgtacctgcagatgaactccctgcgggccgaggacaccgccgtgtactactgtgctagaaccgtgcccggctactccaccgccccttactttgatctgtggggccagggaaccctcgtgaccgtgtctagcggaggcggaggtagtggcggtggtggaagtggcggagggggtagtggtggcgggggatctgatattcagatgacccagtccccctcctccgtgtccgcttccgtgggagatcgcgtgacaatcacatgccagtcctccccctctgtgtggtccaacttcctgtcttggtatcagcagaaacctgggaaggctcctaaactgctgatctatgaggcctccaagctgacctccggcgtgccctctagattctctggcagcggcagcggaaccgactttaccctgaccatcagctccctgcagcctgaagattttgctacctattactgcggtggcggctacagctccatctccgataccaccttcggctgcggaacaaaagtggaaatcaaacgcacctgatagaagcttg

SEQ ID NO: 147 - protein, 107 aa, Artificial Sequence
  Ligh Chain Variable region of antibody CA2109 gL18
DIQMTQSPSSLSASVGDRVTITCRASEDIYSGLAWYQQKPGKVPKLLIYDSSTLHTGVPSRFSGTGSGTDYTLTISSLQPEDVATYFCQQNYDFPLTFGQGTKLEIK

(19) United States
(12) Patent Application Publication
(10) Pub. No.: US 2020/0277366 A1
(43) Pub. Date: Sep. 3, 2020

(54) MULTI-SPECIFIC ANTIBODY MOLECULES HAVING SPECIFICITY FOR TNF-ALPHA, IL-17A and IL-17F

(71) Applicant: UCB BIOPHRMA SPRL, Brussels (BE)

(72) Inventors: Ralph ADAMS, Slough (GB); Pallavi BHATTA, Slough (GB); Emma DAVE, Slough (GB); Sam Philip HEYWOOD, Slough (GB); David Taul HUMPHERYS, Slough (GB); Diane MARSHALL, Slough (GB); Stevan Graham SHAW, Sloug (GB); Daniel John LIGHTWOOD, Slough (GB)

(73) Assignee: UCB Biopharma SPRL, Brussels (BE)

(21) Appl. No.: 16/063,108

(22) PCT Filed: Dec. 14, 2016
(86) PCT No.: PCT/EP2016/080979

§ 371 Date: Jun. 15, 2018

(30) Foreign Application Priority Data
Dec. 18, 2015 (GB) 1522391.0

(51) Int. Cl. C07K 16/24; C12N 15/63

(52) U.S. Cl. CPC C07K 16/241; C07K 16/244; C12N 15/63; C07K 2317/31; C07K 2317/76; A61K 2039/505; C07K 2317/622; C07K 2317/624; C07K 2317/55; C07K 2317/565; C07K 2317/92

(57) ABSTRACT

The invention relates to multi-specific antibody molecules having specificity for TNF alpha, IL-17A and IL-17F, therapeutic uses of the antibody molecules and methods for producing said antibody molecules.

### FIELD OF INVENTION

The present disclosure relates to multi-specific antibody molecules having specificity for human TNF-alpha, human IL-17A and human IL-17F. The present invention also relates to therapeutic uses of the antibody molecules and methods for producing them.

### BACKGROUND OF INVENTION

The role of TNFa as a key driver of disease in rheumatoid arthritis (RA) patients is well established. Indeed, anti-TNFa antibodies have transformed patient care. However, despite this success, there is still a significant unmet medical need as many patients do not achieve clinical remission. Therefore the next clinical ambition for the treatment of RA is to address this unmet clinical need and provide a treatment that significantly improves the proportion of patients that achieve clinical remission. Recent studies have highlighted that there appears to be an enhancement of the Th17/IL-17 biological pathway in RA patients. It has been shown that there is an increase in the percentage of Th17 cells in the peripheral blood of RA patients compared to healthy volunteers and that this is increased further after treatment with anti-TNFa (Alzabin et al., 2012; Chen et al., 2011; Aerts et al., 2010). This enhancement towards a complementary biological pathway after anti-TNFa treatment may partly explain why many patients have only partial responses to therapy or why some patients relapse despite initial responses to treatment.

There is now a body of literature providing evidence for a role of IL-17 in the pathogenesis of RA. The IL-17 family of cytokines consists of 6 members based on structure similarities, with a molecular mass of 23-36 kDa and a dimer structure. The founding member IL-17A (often still referred to in the literature as simply IL-17) shares 16%-50% amino acid sequence identity with other members: IL-17B, IL-17C, IL-17D, IL-17E (also known as IL-25) and IL-17F. IL-17A and IL-17F share the greatest homology (50%) and bind to the same receptor complex thus shared biological activities have been noted between these 2 cytokines. In addition, IL-17A and IL-17F exist not only as homodimers, but also as an IL-17A/F heterodimer. IL-17E (IL-25) has the least similarity with IL-17A. Of significance and relevance to the biological activity of IL-17A and IL-17F is the finding that they share the same IL-17RA/IL-17RC receptor complex, with IL-17A having greatest affinity for IL-17RA, whereas IL-17F binds more strongly to IL-17RC. The other family member to utilise IL-17RA is IL-17E, which signals via the IL-17RA/IL-17RB receptor complex.

IL-17A and IL-17F are produced by the Th17 subset of CD4+ T cells. In addition, other T cell subsets produce IL-17A and IL-17F including cytotoxic CD8+ T cells (Tc17), gdT cells and NK T cells. Other cell populations reported to secrete IL-17A include neutrophils, monocytes, NK cells, lymphoid tissue inducer-like (LTi-like) cells, intestinal paneth cells and even B cells and mast cells. In addition, epithelial cells have been reported to secrete IL-17F.

The cell types which respond to IL-17 cytokines are reflected by the expression of the different receptors. IL-17RA is ubiquitously expressed, with particularly high levels in haematopoietic tissues whereas IL-17RC is more highly expressed in non-immune cells of joints, liver, kidney, thyroid and prostate. This differential expression could explain differences in IL-17A and IL-17F biological activity as cells expressing high levels of IL-17RC could be more responsive to IL-17F whereas cells with higher expression of IL17-RA than IL-17RC may respond more readily to IL-17A. Specific cell types that are responsive to IL-17A and F include fibroblasts, epithelial cells, keratinocytes, synoviocytes and endothelial cells with IL-17A also reported to act on T and B cells and macrophages.

IL-17A and IL-17F are inducers of proinflammatory cytokines, chemokines and matrix metalloproteinases (including IL-6, IL-8 and MMP-13) from fibroblasts, endothelial cells and epithelial cells. IL-17F has often been reported to be less active than IL-17A, however when IL-17F is in combination with TNFa increased biological responses are noted (Zrioual et al., 2009). This additive or synergistic biological activity with TNFa is noted with both IL-17A and IL-17F and may be due to increasing mRNA stability. The expression of both IL-17A and IL-17F has been shown to be increased in RA patients (Zrioual et al., 2009) with the contribution of T cells and IL-17 in RA pathogenesis now well published in the literature (Hot & Miossec 2011; Truchetet et al., 2013). Work with human synovial and bone explants has demonstrated IL-17A to increase cartilage and bone degradation (Chabaud et al., 2001). Further studies in the collagen induced arthritis (CIA) model in mice has also demonstrated that over expression of IL-17A induces synovial inflammation and joint destruction and CIA has been shown to be inhibited by IL-17A blockade and in IL-17-deficient mice (Lubberts 2001 & 2004; Nakae 2003).

A treatment whereby both the TNFa and the IL-17 biological pathways are simultaneously blocked therefore has the potential to significantly improve response rates and address the existing unmet need in RA patients and in the treatment of other pathological disorders mediated by TNF-alpha and IL-17A and/or IL-17F.

WO2014/044758 (Covagen AG) discloses a fusion construct capable of inhibiting glycosylated IL-17A and binding TNF-alpha. WO2013/063110 (Abbvie Inc.) discloses a multivalent DVD-Ig binding protein capable of binding TNF and IL-17. WO2014/137961 (Eli Lilly and Company) discloses anti-TNF and anti-IL-17A bispecific antibodies. However, these molecules may only ever achieve limited efficacy as IL-17F is not neutralised and, as noted above, IL-17F confers similar biological activities to IL-17A, thus only partial inhibition of the IL-17 biological pathway can be achieved with these therapeutic molecules. Inhibition of both IL-17A and IL-17F in combination with inhibition of TNF-alpha may provide improved efficacy over TNF-alpha and IL-17A only blockers.

The present inventors have developed a novel multi-specific antibody, which is capable of inhibiting TNF-alpha, IL-17A and IL-17F which will provide a new treatment option for patients.

### SUMMARY OF THE DISCLOSURE

The present invention provides a multi-specific antibody molecule capable of binding TNF-alpha, IL-17A and IL-17F, in particular, comprising a binding domain specific to human TNF-alpha and a binding domain specific to human IL-17A and human IL-17F, wherein the antibody molecule is capable of neutralising the biological activity of human TNF-alpha, human IL-17A and human IL-17F. In one embodiment, the multi-specific antibody molecule is tri-specific and further comprises a binding domain specific to human serum albumin.

### DETAILED DESCRIPTION OF THE INVENTION

“Antigen binding site” or “binding site” as employed herein refers to a portion of the molecule, which comprises a part or the whole of one or more variable domains, for example a part or the whole of a pair of variable domains, that interact specifically with the target antigen.

“Binding domain” as employed herein refers to a portion of the molecule, which comprises one or more variable domains, for example a pair of variable domains VH and VL, that interact specifically with the target antigen and optionally one or more constant domains for example a CH1 domain and/or a CL domain, either kappa or lambda. A binding domain may comprise a single domain antibody. In one embodiment, each binding domain is monovalent. Preferably each binding domain comprises no more than one VH and one VL.

“Specifically” as employed herein is intended to refer to a binding site or binding domain that only recognises the antigen to which it is specific or a binding site or binding domain that has significantly higher binding affinity to the antigen to which is specific compared to the affinity to antigens to which it is non-specific, for example 5, 6, 7, 8, 9, 10 times higher binding affinity.

“Multi-specific antibody” as employed herein refers to an antibody molecule as described herein which has two or more binding domains, for example two or three binding domains.

In one embodiment the construct is a bi-specific antibody.

“Bi-specific antibody” as employed herein refers to an antibody molecule with two antigen binding sites, wherein one binding site binds human TNF-alpha and the other binding site binds human IL-17A and human IL-17F.

In one embodiment the antibody construct is a tri-specific antibody.

“Tri-specific antibody” as employed herein refers to an antibody molecule with three antigen binding sites.

In one embodiment of the present invention, there is provided a multi-specific antibody molecule capable of binding to human TNF-alpha, human IL-17A and human IL-17F, wherein the antibody molecule is capable of neutralising the biological activity of human TNF-alpha, human IL-17A and human IL-17F.

In one embodiment, the multi-specific antibody molecule comprises a first binding domain specific to human TNF-alpha, a second binding domain specific to human IL-17A and a third binding domain specific to human IL-17F.

In an alternative embodiment, the multi-specific antibody molecule comprises a first binding domain specific to human TNF-alpha and a second binding domain specific to both human IL-17A and human IL-17F.

In one embodiment the antibody molecule comprises three binding domains and two binding domains bind the same antigen, including binding the same epitope or different epitopes on the same antigen, and the third binding domain binds a different (distinct) antigen. In one example a multi-specific antibody molecule binds the antigens human TNF-alpha, human IL-17A and human IL-17F wherein two binding domains bind human TNF-alpha and the third binding domain binds human IL-17A and human IL-17F. In another example, a multi-specific antibody molecule binds the antigens human TNF-alpha, human IL-17A and human IL-17F wherein two binding domains are each capable of binding human IL-17A and human IL-17F and the third binding domain binds human TNF-alpha.

The antibody molecule according to the present invention in one embodiment comprises no more than one binding domain which is specific to human TNF-alpha and no more than one binding domain which is specific to human IL-17A and human IL-17F. Accordingly, in this embodiment, the antibody molecule is monovalent for binding to human TNF-alpha and monovalent for binding to human IL-17A and human IL-17F.

In one embodiment the multi-specific antibody molecule of the invention comprises three binding domains which independently bind three different antigens. In one embodiment, the multi-specific antibody molecule binds the antigens human TNF-alpha, human IL-17A and human IL-17F wherein the first binding domain binds human TNF-alpha, the second binding domain binds human IL-17A and the third binding domain binds human IL-17F.

In another embodiment, the multi-specific antibody molecule binds the antigens human TNF-alpha, human IL-17A, human IL-17F and human serum albumin, wherein the first binding domain binds human IL-17A and human IL-17F, the second binding domain binds human TNF-alpha and the third binding domain binds an antigen capable of extending the half-life of the antibody molecule. The third binding domain which binds an antigen capable of extending the half-life of the antibody molecule may bind to a human serum carrier protein, a circulating immunoglobulin molecule, or CD35/CR1.

As used herein, “serum carrier proteins” include thyroxine-binding protein, transthyretin, α1-acid glycoprotein, transferrin, fibrinogen and albumin, or a fragment of any thereof.

As used herein, a “circulating immunoglobulin molecule” includes IgG1, IgG2, IgG3, IgG4, sIgA, IgM and IgD, or a fragment of any thereof.

CD35/CR1 is a protein present on red blood cells which have a half-life of 36 days (normal range of 28 to 47 days; Lanaro et al., 1971, Cancer, 28(3):658-661).

In a specific embodiment, the multi-specific antibody molecule comprises or consists of three binding domains, wherein the first binding domain is specific to human TNF-alpha, the second binding domain is specific to human IL-17A and human IL-17F and the third binding domain is specific to human serum albumin.

The antibody molecule according to the present invention in one embodiment comprises no more than one binding domain which is specific to human TNF-alpha, no more than one binding domain which is specific to human IL-17A and human IL-17F and no more than one binding domain which is specific to a human serum carrier protein, for example human serum albumin. Accordingly, in this embodiment, the antibody molecule is monovalent for binding to human TNF-alpha, monovalent for binding to human IL-17A and human IL-17F and monovalent for binding to a human serum carrier protein, for example human serum albumin.

Antibody molecules comprising multiple binding domains to the same target antigen, wherein said target antigen is a homomultimer, for example human TNF-alpha trimer or human IL-17 dimer, may be more likely to form large antibody and antigen complexes in vivo. In the embodiment of the present invention wherein the antibody molecule comprises no more than one binding domain to each target antigen, the antibody may advantageously have lower propensity to form large antibody and antigen complexes in vivo compared to those antibody molecules comprising multiple binding domains to the same target antigen.

The present invention provides an improved multi-specific antibody which is capable of binding to both IL-17A and IL-17F with high affinity. In particular, the multi-specific antibody of the present invention is capable of specifically binding to both IL-17A and IL-17F. Specifically binding means that the antibodies have a greater affinity for IL-17A and IL-17F polypeptides (including the IL-17A/IL-17F heterodimer) than for other polypeptides and in one embodiment the antibody does not bind to other isoforms of IL-17. The multi-specific antibody of the present invention is capable of specifically binding to the IL-17A homodimer and the IL-17F homodimer. Preferably the multi-specific antibody of the present invention also binds the IL-17A/IL-17F heterodimer.

Preferably, the multi-specific antibody of the present invention neutralises the activity of both IL-17A and IL-17F. In one embodiment the multi-specific antibody of the present invention also neutralises the activity of the IL-17A/IL-17F heterodimer. The multi-specific antibodies of the present invention therefore have the advantageous property that they can inhibit the biological activity of both IL-17A and IL-17F.

As used herein, the term ‘neutralising antibody’ describes an antibody that is capable of neutralising the biological signalling activity of both IL-17A and IL-17F for example by blocking binding of IL-17A and IL-17F to one or more of their receptors and by blocking binding of the IL-17A/IL-17F heterodimer to one or more of its receptors. It will be appreciated that the term ‘neutralising’ as used herein refers to a reduction in biological signalling activity which may be partial or complete. Further, it will be appreciated that the extent of neutralisation of IL-17A and IL-17F activity by the antibody may be the same or different. In one embodiment the extent of neutralisation of the activity of the IL-17A/IL-17F heterodimer may be the same or different as the extent of neutralisation of IL-17A or IL-17F activity. Suitable assays for determining neutralisation are known in the art and certain of such assays are provided in the Examples herein.

Preferably the IL-17A and IL-17F polypeptides are human. In one embodiment the antibody also binds cynomolgus IL-17A and IL-17F.

The multi-specific antibody is also advantageously capable of binding to TNF-alpha with high affinity. In particular, the multi-specific antibody of the present invention is capable of specifically binding to TNF-alpha.

Preferably, the multi-specific antibody of the present invention neutralises the activity of TNF-alpha, for example by blocking binding of TNF-alpha to one or more receptors of TNF-alpha. It will be appreciated that the term ‘neutralising’ as used herein refers to a reduction in biological signalling activity which may be partial or complete. Preferably the TNF-alpha polypeptide is human. In one embodiment the antibody also binds cynomolgus TNF-alpha. Suitable assays for determining neutralisation are known in the art and certain of such assays are provided in the Examples herein.

Accordingly, the present invention also provides the use of such antibodies in the treatment of and/or prophylaxis of a disease mediated by TNF-alpha and IL-17A and/or IL-17F such as autoimmune or inflammatory disease or cancer.

Binding affinity (KD) may be measured by standard assay, for example surface plasmon resonance, such as BIAcore.

In one embodiment the multi-specific antibody molecule has a binding affinity for human TNF-alpha of 200 pM or less, for example 100 pM or better, 50 pM or better, 20 pM or better, 15 pM or better or 12 pM or better. In one embodiment the multi-specific antibody molecule has a binding affinity for human TNF-alpha in the range of 50 pM to 1 pM, 20 pM to 1 pM, 15 pM to 1 pM or 12 pM to 1 pM. In one embodiment, the multi-specific antibody molecule has a binding affinity for human TNF-alpha in the range of 15 pM to 5 pM or 12 pM to 11 pM.

In one embodiment the multi-specific antibody molecule has a binding affinity for human for human IL-17A of 200 pM or less, for example 100 pM or better, 50 pM or better, 20 pM or better, 10 pM or better, 8 pM or better, 5 pM or better or 2 pM or better. In one embodiment the multi-specific antibody molecule has a binding affinity for human IL-17A in the range of 50 pM to 1 pM, 20 pM to 1 pM, 10 pM to 1 pM, 8 pM to 1 pM, 5 pM to 1 pM or 2 pM to 1 pM.

In one embodiment the multi-specific antibody molecule has a binding affinity for human IL-17F of 200 pM or less, for example 100 pM or better, 50 pM or better, 20 pM or better, 15 pM or better or 10 pM or better. In one embodiment the multi-specific antibody molecule has a binding affinity for human IL-17F in the range of 50 pM to 1 pM, 20 pM to 1 pM, 15 pM to 1 pM or 10 pM to 1 pM. In one embodiment, the multi-specific antibody molecule has a binding affinity for human IL-17F in the range of 10 pM to 5 pM or 8 pM to 7 pM.

In one embodiment the multi-specific antibody molecule has a binding affinity for human serum albumin of 3 nM or better, 2 nM or better, 1.9 nM or better or 1.8 nM or better. In one embodiment the multi-specific antibody molecule has a binding affinity for human serum albumin in the range of 3 nM to 1 pM, 2 nM to 1 nM, 2 nM to 1.5 nM or 1.8 nM to 1.7 nM.

In one aspect of the present invention, each binding domain comprises two antibody variable domains, preferably a VH/VL pair. In one aspect each binding domain comprises no more than two antibody variable domains.

The multispecific antibody molecule of the present invention may have any suitable antibody format which is capable of binding two or more antigens, for example three antigens, as described above.

In one aspect, the antibody molecule format is selected from diabody, scdiabody, triabody, tandem scFv, FabFv, Fab′Fv, FabdsFv, Fab-scFv, Fab-dsscFv, Fab-(dsscFv)2, FabFvFv, FabFvFc, diFab, diFab′, tribody, tandem scFv-Fc, scFv-Fc-scFv, scdiabody-Fc, scdiabody-CH3, Ig-scFv, scFv-Ig, V-Ig, Ig-V, Duobody and DVD-Ig. In one example the antibody molecule has the format illustrated in FIG. 8 as 2xdsscFv and described in WO2015/197772.

In one embodiment, the binding domain which is specific to human TNF-alpha and the binding domain which is specific to IL-17A and human IL-17F are independently selected from a Fab, scFv, Fv, dsFv and dsscFv.

In one embodiment of the present invention, the antibody molecule does not comprise a CH2 domain and/or a CH3 domain. An antibody molecule which lacks a CH2 domain and/or a CH3 domain, also referred to as a Fc domain, is advantageous where no functional properties attributed to the Fc domain, such as complement binding, are required.

The presence of an Fc domain, particularly an active Fc domain such as an IgG1 isotype Fc, in antibody therapeutics can lead to interactions with pro-inflammatory proteins such as FcGR and complement in vivo. Accordingly, in the embodiment wherein the antibody molecule of the present invention does not comprise an Fc domain, the antibody may have fewer interactions with pro-inflammatory proteins such as FcGR and complement in vivo compared to antibody molecules containing an Fc domain.

Antibodies comprising both multiple binding domains to the same target antigen, wherein said target antigen is a homomultimer, and Fc domains may combine the propensity to form large antibody:antigen complexes with multiple active Fc domains to form large immune complexes.

Large immune complexes may be inappropriately deposited in vivo leading to immune system activation. In the embodiment of the present invention wherein the antibody comprises no more than one binding domain to each target antigen and does not comprise an Fc domain, the antibody may have a reduced ability to form large immune complexes and therefore a lower likelihood of inappropriate deposition and immune activation in vivo.

In one aspect of the present invention, the multi-specific antibody molecule is provided as a dimer comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} represents a heavy chain variable domain;
CH₁ represents a domain of a heavy chain constant region, for example domain 1 thereof;
X represents a bond or linker;
Y represents a bond or linker;
V₁ represents a dsFv, a sdAb, a scFv or a dsscFv;
V_{L1} represents a light chain variable domain;
C_{L} represents a domain from a light chain constant region, such as Ckappa;
V₂ represents dsFv, a sdAb, a scFv or a dsscFv.

In one embodiment V_{H1} and V_{L1} together form a binding domain specific to a first antigen selected from human TNF-alpha, human IL-17A and human IL-17F. In one embodiment, VH1 and VL1 together form a binding domain specific to human IL-17A and human IL-17F.

In one embodiment V₁ comprises a binding domain specific to a second antigen selected from human TNF-alpha, human IL-17A and human IL-17F. In one embodiment, V1 comprises a binding domain specific to human IL-17A and human IL-17F.

In one embodiment V₂ comprises a binding domain specific to a second or third antigen selected from human TNF-alpha, human IL-17A and human IL-17F. In one embodiment, V2 comprises a binding domain specific to human IL-17A and human IL-17F.

In one embodiment, VH1 and VL1 comprise a binding domain specific to human IL-17A and human IL-17F and V1 and/or V2 comprise a binding domain specific to human TNF-alpha. In the embodiment wherein V1 and V2 comprise a binding domain specific to human TNF-alpha, V1 and V2 may bind the same or a different epitope of human TNF-alpha.

In one embodiment, VH1 and VL1 comprise a binding domain specific to human TNF-alpha and V1 and/or V2 comprise a binding domain specific to human IL-17A and human IL-17F. In the embodiment wherein V1 and V2 comprise a binding domain specific to human IL-17A and human IL-17F, V1 and V2 may bind the same or a different epitope of human IL-17A and human IL-17F.

In one embodiment, the antibody molecule comprises or consists of the polypeptide chains as defined in formula (I) and formula (II) and comprises no more than one binding domain which is specific to human TNF-alpha and no more than one binding domain which is specific to human IL-17A and human IL-17F.

In one aspect of the present invention, the multi-specific antibody molecule is capable of binding to a further antigen and comprises a binding domain specific to a serum carrier protein, a circulating immunoglobulin molecule, or CD35/CR1, for example for providing an extended half-life to the antibody molecule.

In one embodiment, the antigen of interest for which V_{H1}/V_{L1} has specificity is a serum carrier protein, such as a human serum carrier, such as human serum albumin.

In one embodiment, the antigen of interest for which V₁ has specificity is a serum carrier protein, such as a human serum carrier, such as human serum albumin.

In one embodiment, the antigen of interest for which V₂ has specificity is a serum carrier protein, such as a human serum carrier, such as human serum albumin.

In one embodiment only one of V_{H1}/V_{L1}, V₁ or V₂ has specificity for a serum carrier protein, such as a human serum carrier, such as human serum albumin.

Accordingly, in one embodiment, the antibody molecule comprises or consists of the polypeptide chains as defined in formula (I) and formula (II) above, wherein:
a. VH1 and VL1 comprise the binding domain specific to human IL-17A and human IL-17F; and
b. V1 comprises the binding domain specific to human TNF-alpha and V2 comprises a binding domain specific to a serum carrier protein, for example human serum albumin; or V2 comprises the binding domain specific to human TNF-alpha and V1 comprises a binding domain specific to a serum carrier protein, for example human serum albumin.

In one embodiment, the antibody molecule comprises or consists of the polypeptide chains as defined in formula (I) and formula (II) and comprises no more than one binding domain which is specific to human TNF-alpha, no more than one binding domain which is specific to human IL-17A and human IL-17F and no more than one binding domain which is specific to a serum carrier protein, for example human serum albumin.

The V_{H1}-CH₁ portion together with the V_{L}-C_{L} portion form a functional Fab or Fab′ fragment.

V_{H1} represents a variable domain, for example a heavy chain variable domain. In one embodiment V_{H1} represents a heavy chain variable domain. In one embodiment V_{H1} is a chimeric variable domain, that is to say it comprises components derived from at least two species, for example a human framework and non-human CDRs. In one embodiment V_{H1} is humanised. In one embodiment the V_{H1} is human.

V_{L1} represents a variable domain, for example a light chain variable domain. In one embodiment V_{L1} represents a light chain variable domain. In one embodiment V_{L1} is a chimeric variable domain, that is to say it comprises components derived from at least two species, for example a human framework and non-human CDRs. In one embodiment V_{L1} is humanised. In one embodiment V_{L1} is humanised. In one embodiment the V_{L} is human.

Generally V_{H1} and V_{L1} together form an antigen binding domain. In one embodiment V_{H1} and V_{L1} form a cognate pair.

“Cognate pair” as employed herein refers to a pair of variable domains from a single antibody, which was generated in vivo, i.e. the naturally occurring pairing of the variable domains isolated from a host. A cognate pair is therefore a V_{H} and V_{L} pair. In one example the cognate pair bind the antigen co-operatively.

“Variable region” as employed herein refers to the region in an antibody chain comprising the CDRs and a framework, in particular a suitable framework.

Variable regions for use in the present disclosure will generally be derived from an antibody, which may be generated by any method known in the art.

“Derived from” as employed herein refers to the fact that the sequence employed or a sequence highly similar to the sequence employed was obtained from the original genetic material, such as the light or heavy chain of an antibody.

“Highly similar” as employed herein is intended to refer to an amino acid sequence which over its full length is 95% similar or more, such as 96, 97, 98 or 99% similar.

Variable regions for use in the present invention, as described herein above for V_{H1} and V_{L1} may be from any suitable source and may be for example, fully human or humanised.

In one embodiment the CH₁ domain is a naturally occurring domain 1 from an antibody heavy chain or a derivative thereof.

In one embodiment the C_{L} fragment, in the light chain, is a constant kappa sequence or a constant lambda sequence or a derivative thereof.

A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

In one embodiment one or more natural or engineered inter chain (i.e. inter light and heavy chain) disulphide bonds are present in the functional Fab or Fab′ fragment.

In one embodiment a “natural” disulfide bond is present between a CH₁ and C_{L} in the polypeptide chains of Formula (I) and (II).

When the C_{L} domain is derived from either Kappa or Lambda the natural position for a bond forming cysteine is 214 in human cKappa and cLambda (Kabat numbering 4^{th} edition 1987).

The exact location of the disulfide bond forming cysteine in CH₁ depends on the particular domain actually employed. Thus, for example in human gamma-1 the natural position of the disulfide bond is located at position 233 (Kabat numbering 4^{th} edition 1987). The position of the bond forming cysteine for other human isotypes such as gamma 2, 3, 4, IgM and IgD are known, for example position 127 for human IgM, IgE, IgG2, IgG3, IgG4 and 128 of the heavy chain of human IgD and IgA2B.

Optionally there may be a disulfide bond between the VH and V_{L} of the polypeptides of formula I and II.

In one embodiment the multi-specific antibody according to the disclosure has a disulfide bond in a position equivalent or corresponding to that naturally occurring between CH₁ and C_{L}.

In one embodiment a constant region comprising CH₁ and a constant region such as C_{L} has a disulfide bond which is in a non-naturally occurring position. This may be engineered into the molecule by introducing cysteine(s) into the amino acid chain at the position or positions required. This non-natural disulfide bond is in addition to or as an alternative to the natural disulfide bond present between CH₁ and C_{L}. The cysteine(s) in natural positions can be replaced by an amino acid such as serine which is incapable on forming a disulfide bridge.

Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, N Y, 1989; Ausbel et al., Current Protocols in Molecular Biology, Greene Publishing & Wiley-Interscience, N Y, 1993). Site-directed mutagenesis kits are commercially available, e.g. QuikChange® Site-Directed Mutagenesis kit (Stratagene, La Jolla, Calif.). Cassette mutagenesis can be performed based on Wells et al., 1985, Gene, 34:315-323. Alternatively, mutants can be made by total gene synthesis by annealing, ligation and PCR amplification and cloning of overlapping oligonucleotides.

In one embodiment a disulfide bond between CH₁ and C_{L} is completely absent, for example the interchain cysteines may be replaced by another amino acid, such as serine. Thus in one embodiment there are no interchain disulphide bonds in the functional Fab fragment of the molecule. Disclosures such as WO2005/003170, incorporated herein by reference, describe how to provide Fab fragments without an inter chain disulphide bond.

The antibody molecule as defined above comprising or consisting of the polypeptide chains as defined in formula (I) and formula (II) in one embodiment does not comprise a CH2 domain and/or a CH3 domain.

V₁ represents a dsFv, a sdAb, a scFv, or a dsscFv, for example a dsFv, scFv or a dsscFv.

V₂ represents a dsFv, a sdAb, a scFv, or a dsscFv, for example a dsFv, scFv or a dsscFv.

“Single chain variable fragment” or “scFv” as employed herein refers to a single chain variable fragment comprising or consisting of a heavy chain variable domain (V_{H}) and a light chain variable domain (V_{L}) which is stabilised by a peptide linker between the V_{H} and V_{L} variable domains. The V_{H} and V_{L} variable domains may be in any suitable orientation, for example the C-terminus of V_{H} may be linked to the N-terminus of V_{L} or the C-terminus of V_{L} may be linked to the N-terminus of V_{H}.

“Disulphide-stabilised single chain variable fragment” or “dsscFv” as employed herein refers to a single chain variable fragment which is stabilised by a peptide linker between the V_{H} and V_{L} variable domain and also includes an inter-domain disulphide bond between V_{H} and V_{L}.

“Disulphide-stabilised variable fragment” or “dsFv” as employed herein refers to a single chain variable fragment which does not include a peptide linker between the V_{H} and V_{L} variable domains and is instead stabilised by an interdomain disulphide bond between V_{H} and V_{L}.

“Single domain antibody” or “sdAb” as employed herein refers to an antibody fragment consisting of a single monomeric variable antibody domain, such as V_{H} or V_{L} or VHH.

In one embodiment, V₁ and V₂ are both dsFv. When both V₁ and V₂ are dsFv, either the V_{H} or the V_{L} variable domains are the same for V₁ and V₂. In one embodiment, V₁ and V₂ have the same V_{H} variable domain. In another embodiment, V₁ and V₂ have the same V_{L} variable domain.

In one embodiment the V_{H} and V_{L} variable domains are the same for V₁ and V₂. The latter allows for cross-linking which may be desirable for some targets.

In one embodiment V₁ is a dsFv and V₂ is a scFv. In one embodiment V₁ is a scFv and V₂ is a dsFv. In one embodiment V₁ is a dsscFv and V₂ is a dsFv. In one embodiment V₁ is a dsFv and V₂ is a dsscFv. In one embodiment V₁ is a dsscFv and V₂ is a scFv. In one embodiment V₁ is a scFv and V₂ is a dsscFv. In one embodiment, V₁ is not a scFv. In one embodiment, V₂ is not a scFv. In one embodiment, both V₁ and V₂ are not scFv.

In the embodiment wherein V₁ and/or V₂ are a dsFv or a dsscFv, the light chain and heavy chain variable domains of V₁ and/or the light chain and heavy chain variable domains of V₂ are linked by a disulfide bond between two engineered cysteine residues. The disulfide bond between the variable domains V_{H} and V_{L} of V₁ and/or V₂ is between two of the residues listed below (unless the context indicates otherwise Kabat numbering is employed in the list below). Wherever reference is made to Kabat numbering the relevant reference is Kabat et al., 1987, in Sequences of Proteins of Immunological Interest, US Department of Health and Human Services, NIH, USA.

In one embodiment the disulfide bond is in a position selected from the group comprising:
V_{H}37+V_{L}95, see for example Protein Science 6, 781-788 Zhu et al (1997);
V_{H}44+V_{L}100, see for example; Biochemistry 33 5451-5459 Reiter et al (1994); or Journal of Biological Chemistry Vol. 269 No. 28 pp. 18327-18331 Reiter et al (1994); or Protein Engineering, vol. 10 no. 12 pp. 1453-1459 Rajagopal et al (1997);
V_{H}44+V_{L}105, see for example J Biochem. 118, 825-831 Luo et al (1995);
V_{H}45+V_{L}87, see for example Protein Science 6, 781-788 Zhu et al (1997);
V_{H}55+V_{L}101, see for example FEBS Letters 377 135-139 Young et al (1995);
V_{H}100+V_{L}50, see for example Biochemistry 29 1362-1367 Glockshuber et al (1990);
V_{H}100b+V_{L}49;
V_{H}98+V_{L} 46, see for example Protein Science 6, 781-788 Zhu et al (1997);
V_{H}101+V_{L}46;
V_{H}105+V_{L}43, see for example; Proc. Natl. Acad. Sci. USA Vol. 90 pp. 7538-7⁵⁴² Brinkmann et al (1993); or Proteins 19, 35-47 Jung et al (1994),
V_{H}106+V_{L}57, see for example FEBS Letters 377 135-139 Young et al (1995) wherein the V_{H} and V_{L} values are independently selected within a given V₁ or V₂.

The amino acid pairs listed above are in the positions conducive to replacement by cysteines such that disulfide bonds can be formed. Cysteines can be engineered into these desired positions by known techniques. In one embodiment therefore an engineered cysteine according to the present disclosure refers to where the naturally occurring residue at a given amino acid position has been replaced with a cysteine residue.

Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, N Y, 1989; Ausbel et al., Current Protocols in Molecular Biology, Greene Publishing & Wiley-Interscience, N Y, 1993). Site-directed mutagenesis kits are commercially available, e.g. QuikChange® Site-Directed Mutagenesis kit (Stratagen, La Jolla, Calif.). Cassette mutagenesis can be performed based on Wells et al., 1985, Gene, 34:315-323. Alternatively, mutants can be made by total gene synthesis by annealing, ligation and PCR amplification and cloning of overlapping oligonucleotides.

Accordingly, in one embodiment when V₁ and/or V₂ are a dsFv or a dsscFv, the variable domains V_{H} and V_{L} of V₁ and/or the variable domains V_{H} and V_{L} of V₂ may be linked by a disulfide bond between two cysteine residues, wherein the position of the pair of cysteine residues is selected from the group consisting of: V_{H}37 and V_{L}95, V_{H}44 and V_{L}100, V_{H}44 and V_{L}105, V_{H}45 and V_{L}87, VH100 and V_{L}50, V_{H}100b and V_{L}49, V_{H}98 and V_{L}46, V_{H}101 and V_{L}46, V_{H}105 and V_{L}43 and V_{H}106 and V_{L}57.

In one embodiment when V₁ and/or V₂ are a dsFv or a dsscFv, the variable domains V_{H} and V_{L} of V₁ and/or the variable domains V_{H} and V_{L} of V₂ may be linked by a disulfide bond between two cysteine residues, one in V_{H} and one in V_{L}, which are outside of the CDRs wherein the position of the pair of cysteine residues is selected from the group consisting of V_{H}37 and V_{L}95, V_{H}44 and V_{L}100, V_{H}44 and V_{L}105, V_{H}45 and V_{L}87, V_{H}100 and V_{L}50, V_{H}98 and V_{L}46, V_{H}105 and V_{L}43 and V_{H}106 and V_{L}57.

In one embodiment when V₁ is a dsFv or a dsscFv, the variable domains V_{H} and V_{L} of V₁ are linked by a disulphide bond between two engineered cysteine residues, one at position V_{H}44 and the other at V_{L}100.

In one embodiment when V₂ is a dsFv or a dsscFv, the variable domains V_{H} and V_{L} of V₂ are linked by a disulphide bond between two engineered cysteine residues, one at position V_{H}44 and the other at V_{L}100.

In one embodiment when V₁ and V₂ is a dsFv or a dsscFv, the variable domains V_{H} and V_{L} of V₁ and V₂ are linked by a disulphide bond between two engineered cysteine residues, one at position V_{H}44 and the other at V_{L}100.

In one embodiment, when V1 and V2 are both dsscFv the variable domains V_{H} and V_{L} of V₁ and V₂ are linked by a disulphide bond between two engineered cysteine residues, one at position V_{H}44 and the other at V_{L}100.

In one embodiment when V₁ is a dsFv, a dsscFv, or a scFv, the V_{H} domain of V₁ is attached to X, for example through a peptide bond.

In one embodiment when V₁ is a dsFv, a dsscFv, or a scFv, the V_{L} domain of V₁ is attached to X, for example through a peptide bond.

In one embodiment when V₂ is a dsFv, a dsscFv, or a scFv, the V_{H} domain of V₂ is attached to Y, for example through a peptide bond.

In one embodiment when V₂ is a dsFv, a dsscFv, or a scFv, the V_{L} domain of V₂ is attached to Y, for example through a peptide bond.

The skilled person will appreciate that when V₁ and/or V₂ represents a dsFv, the multi-specific antibody will comprise a third polypeptide encoding the corresponding free V_{H} or V_{L} domain which is not attached to X or Y. When V₁ and V₂ are both a dsFv then the “free variable domain” (i.e. the domain linked to via a disulphide bond to the remainder of the polypeptide) will be common to both chains. Thus whilst the actual variable domain fused or linked via X or Y to the polypeptide may be different in each polypeptide chain the free variable domains paired therewith will generally be identical to each other.

In one embodiment X is a bond.

In one embodiment Y is a bond.

In one embodiment both X and Y are bonds.

In one embodiment X is a linker, preferably a peptide linker, for example a suitable peptide for connecting the portions CH₁ and V₁.

In one embodiment Y is a linker, preferably a peptide linker, for example a suitable peptide for connecting the portions C_{L} and V₂.

In one embodiment both X and Y are linkers. In one embodiment both X and Y are peptide linkers.

The term “peptide linker” as used herein refers to a peptide comprised of amino acids. A range of suitable peptide linkers will be known to the person of skill in the art.

In one embodiment the X and/or Y peptide linker is 50 amino acids in length or less, for example 20 amino acids or less, such as about 15 amino acids or less, such as 9, 10, 11, 12, 13 or 14 amino acids in length.

In one embodiment the X and/or Y linker is selected from a sequence shown in sequence 1 to 67.

In one embodiment the X and/or Y linker is selected from a sequence shown in SEQ ID NO: 1 or SEQ ID NO: 2.

(SEQ ID NO: 1)
In one embodiment X has the sequence SGGGGTGGGGS.
(SEQ ID NO: 1)
In one embodiment Y has the sequence SGGGGTGGGGS.
(SEQ ID NO: 2)
In one embodiment X has the sequence SGGGGSGGGGS.
(SEQ ID NO: 2)
In one embodiment Y has the sequence SGGGGSGGGGS.

In one embodiment X has the sequence given in SEQ ID NO:1 and Y has the sequence given in SEQ ID NO:2.

In one embodiment X has the sequence given in SEQ ID NO:2 and Y has the sequence given in SEQ ID NO: 1.

In one embodiment X has the sequence given in SEQ ID NO: 2 and Y has the sequence given in SEQ ID NO:2.

In one embodiment X has the sequence given in SEQ ID NO:69 or 70. In one embodiment Y has the sequence given in SEQ ID NO:69 or 70. In one embodiment X has the sequence given in SEQ ID NO:69 and Y has the sequence given in SEQ ID NO:70.

Suitable linker sequences for X and/or Y are also provided in Tables 1 and 2 below.

TABLE 1
Hinge linker sequences

| SEQ ID NO: | SEQUENCE |
|---|---|
| 3 | DKTHTCAA |
| 4 | DKTHTCPPCPA |
| 5 | DKTHTCPPCPATCPPCPA |
| 6 | DKTHTCPPCPATCPPCPATCPPCPA |
| 7 | DKTHTCPPCPAGKPTLYNSLVMSDTAGTCY |
| 8 | DKTHTCPPCPAGKPTHVNVSVVMAEVDGTCY |
| 9 | DKTHTCCVECPPCPA |
| 10 | DKTHTCPRCPEPKSCDTPPPCPRCPA |
| 11 | DKTHTCPSCPA |

TABLE 2
Flexible linker sequences

| SEQ ID NO: | SEQUENCE |
|---|---|
| 12 | SGGGGSE |
| 13 | DKTHTS |
| 14 | (S)GGGGS |
| 15 | (S)GGGGSGGGGS |
| 16 | (S)GGGGSGGGGSGGGGS |
| 17 | (S)GGGGSGGGGSGGGGSGGGGS |
| 18 | (S)GGGGSGGGGSGGGGSGGGGSGGGGS |
| 19 | AAAGSG-GASAS |
| 20 | AAAGSG-XGGGS-GASAS |
| 21 | AAAGSG-XGGGSXGGGS -GASAS |
| 22 | AAAGSG- XGGGSXGGGSXGGGS -GASAS |
| 23 | AAAGSG- XGGGSXGGGSXGGGSXGGGS-GASAS |
| 24 | AAAGSG-XS-GASAS |
| 25 | PGGNRGTTTTRRPATTTGSSPGPTQSHY |
| 26 | ATTTGSSPGPT |
| 27 | ATTTGS |
| — | GS |
| 28 | EPSGPISTINSPPSKESHKSP |
| 29 | GTVAAPSVFIFPPSD |
| 30 | GGGGIAPSMVGGGGS |
| 31 | GGGGKVEGAGGGGGS |
| 32 | GGGGSMKSHDGGGGS |
| 33 | GGGGNLITIVGGGGS |
| 34 | GGGGVVPSLPGGGGS |
| 35 | GGEKSIPGGGGS |
| 36 | RPLSYRPPFPFGFPSVRP |
| 37 | YPRSIYIRRRHPSPSLTT |
| 38 | TPSHLSHILPSFGLPTFN |
| 39 | RPVSPFTFPRLSNSWLPA |
| 40 | SPAAHFPRSIPRPGPIRT |
| 41 | APGPSAPSHRSLPSRAFG |
| 42 | PRNSIHFLHPLLVAPLGA |
| 43 | MPSLSGVLQVRYLSPPDL |
| 44 | SPQYPSPLTLTLPPHPSL |
| 45 | NPSLNPPSYLHRAPSRIS |
| 46 | LPWRTSLLPSLPLRRRP |
| 47 | PPLFAKGPVGLLSRSFPP |
| 48 | VPPAPVVSLRSAHARPPY |
| 49 | LRPTPPRVRSYTCCPTP- |
| 50 | PNVAHVLPLLTVPWDNLR |
| 51 | CNPLLPLCARSPAVRTFP |

(S) is optional in sequences 14 to 18.

Examples of rigid linkers include the peptide sequences GAPAPAAPAPA (SEQ ID NO: 52), PPPP (SEQ ID NO: 53) and PPP.

In one embodiment the peptide linker is an albumin binding peptide.

Examples of albumin binding peptides are provided in WO2007/106120 and include:

TABLE 3

| SEQ ID NO: | SEQUENCE |
|---|---|
| 54 | DLCLRDWGCLW |
| 55 | DICLPRWGCLW |
| 56 | MEDICLPRWGCLWGD |
| 57 | QRLMEDICLPRWGCLWEDDE |
| 58 | QGLIGDICLPRWGCLWGRSV |
| 59 | QGLIGDICLPRWGCLWGRSVK |
| 60 | EDICLPRWGCLWEDD |
| 61 | RLMEDICLPRWGCLWEDD |
| 62 | MEDICLPRWGCLWEDD |
| 63 | MEDICLPRWGCLWED |
| 64 | RLMEDICLARWGCLWEDD |
| 65 | EVRSFCTRWPAEKSCKPLRG |
| 66 | RAPESFVCYWETICFERSEQ |
| 67 | EMCYFPGICWM |

Advantageously use of albumin binding peptides as a linker may increase the half-life of the multi-specific antibody molecule.

In one embodiment at least one of V₁ and V₂ is a scFv or dsscFv. For example, V₁ is a scFv or dsscFv and V₂ is a scFv or dsscFv.

In one embodiment, V₁ is a dsscFv and V₂ is a dsscFv.

In the embodiments wherein V1 is a scFv or dsscFv, V1 comprises or consists of a polypeptide chain of:
a. formula (III): VH2-Z1-VL2 and VH2 is attached to X, for example through a peptide bond; or
b. formula (IV): VL2-Z1-VH2 and VL2 is attached to X, for example through a peptide bond;
   wherein VH2 represents a heavy chain variable domain, Z1 represents a linker, for example a peptide linker and VL2 represents a light chain variable domain.

In the embodiments wherein V2 is a scFv or dsscFv, V2 comprises or consists of a polypeptide chain of:
a. formula (V): VH3-Z2-VL3 and VH3 is attached to Y, for example through a peptide bond; or
b. formula (VI): VL3-Z2-VH3 and VL3 is attached to Y, for example through a peptide bond;
   wherein VH3 represents a heavy chain variable domain, Z2 represents a linker, for example a peptide linker and VL3 represents a light chain variable domain.

In one embodiment the peptide linker Z1 and/or Z2 in the scFv or dsscFv is in range about 12 to 25 amino acids in length, such as 15 to 20 amino acids.

In one embodiment when V₁ is a scFv or a dsscFv, the linker Z1 connecting the variable domains V_{H} and V_{L} of V₁ has the sequence GGGGSGGGGSGGGGSGGGGS (SEQ ID NO: 68).

In one embodiment when V₂ is a scFv or a dsscFv, the linker Z2 connecting the variable domains V_{H} and V_{L} of V₂ has the sequence GGGGSGGGGSGGGGSGGGGS (SEQ ID NO: 68).

In one embodiment when V₁ is a scFv or a dsscFv, the linker Z1 connecting the variable domains V_{H} and V_{L} of V₁ has the sequence SGGGGSGGGGSGGGGS (SEQ ID NO: 69).

In one embodiment when V₂ is a scFv or a dsscFv, the linker Z2 connecting the variable domains V_{H} and V_{L} of V₂ has the sequence SGGGGSGGGGSGGGGS (SEQ ID NO: 69)

In one embodiment when V₁ is a scFv or a dsscFv, the linker Z1 connecting the variable domains V_{H} and V_{L} of V₁ has the sequence SGGGGSGGGGTGGGGS (SEQ ID NO: 70).

In one embodiment when V₂ is a scFv or a dsscFv, the linker Z2 connecting the variable domains V_{H} and V_{L} of V₂ has the sequence SGGGGSGGGGTGGGGS SEQ ID NO: 70).

Accordingly, in one aspect there is provided a multi-specific antibody molecule, comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} represents a heavy chain variable domain;
CH₁ represents a domain of a heavy chain constant region, for example domain 1 thereof;
X represents a linker having the sequence given in SEQ ID NO: 2;
Y represents a linker having the sequence given in SEQ ID NO: 2;
V₁ represents a dsscFv comprising or consisting of a polypeptide chain of formula (III): VH2-Z1-VL2 wherein VH2 represents a heavy chain variable domain, Z represents a linker having the sequence given in SEQ ID NO: 68, VL2 represents a light chain variable domain, VH2 is attached to X, for example through a peptide bond, and the variable domains VH2 and VL2 of V₁ are linked by a disulphide bond between two engineered cysteine residues, one at position VH44 and the other at VL100;
V_{L1} represents a light chain variable domain;
C_{L} represents a domain from a light chain constant region, such as Ckappa;
V₂ represents a dsscFv comprising or consisting of a polypeptide chain of formula (V): VH3-Z2-VL3 wherein VH3 represents a heavy chain variable domain, Z2 represents a linker having the sequence given in SEQ ID NO: 68, VL3 represents a light chain variable domain, VH3 is attached to Y, for example through a peptide bond, and the variable domains VH3 and VL3 of V2 are linked by a disulphide bond between two engineered cysteine residues, one at position VH44 and the other at VL100;
wherein VH1 and VL1 comprise a binding domain specific to human IL-17A and human IL-17F; and V1 comprises a binding domain specific to human TNF-alpha and V2 comprises a binding domain specific to human serum albumin; or V2 comprises the binding domain specific to human TNF-alpha and V1 comprises a binding domain specific to human serum albumin.

The present invention provides a multi-specific antibody molecule as defined above, wherein the binding domain specific for human TNF-alpha comprises at least one of a CDR having the sequence given in SEQ ID NO:85 for CDRH1, a CDR having the sequence given in SEQ ID NO:86 for CDRH2 and a CDR having the sequence given in SEQ ID NO:87 for CDRH3. In one embodiment, the binding domain specific for human TNF-alpha comprises 3 heavy chain CDRs having the sequence given in SEQ ID NO:85 for CDRH1, SEQ ID NO:86 for CDRH2 and SEQ ID NO:87 for CDRH3. In one embodiment, the binding domain specific for human TNF-alpha comprises 3 heavy chain CDRs and the sequence of CDRH1 has at least 60% identity or similarity to the sequence given in SEQ ID NO: 85, the sequence of CDRH2 has at least 60% identity or similarity to the sequence given in SEQ ID NO:86 and the sequence of CDRH3 has at least 60% identity or similarity to the sequence given in SEQ ID NO:87.

The present invention provides a multi-specific antibody molecule as defined above, wherein the binding domain specific for human TNF-alpha comprises at least one of a CDR having the sequence given in SEQ ID NO: 88 or SEQ ID NO: 136 for CDRL1, a CDR having the sequence given in SEQ ID NO:89, SEQ ID NO: 137, SEQ ID NO: 138 or SEQ ID NO: 139 for CDRL2 and a CDR having the sequence given in SEQ ID NO:90 for CDRL3. In one embodiment, the binding domain specific for human TNF-alpha comprises 3 light chain CDRs having the sequence given in SEQ ID NO:88 or SEQ ID NO: 136 for CDRL1, SEQ ID NO:89, SEQ ID NO: 137, SEQ ID NO: 138 or SEQ ID NO: 139 for CDRL2 and SEQ ID NO:90 for CDRL3. In one embodiment, the binding domain specific for human TNF-alpha comprises 3 light chain CDRs and the sequence of CDRL1 has at least 60% identity or similarity to the sequence given in SEQ ID NO:88 or SEQ ID NO: 136, the sequence of CDRL2 has at least 60% identity or similarity to the sequence given in SEQ ID NO:89, SEQ ID NO: 137, SEQ ID NO: 138 or SEQ ID NO: 139 and the sequence of CDRL3 has at least 60% identity or similarity to the sequence given in SEQ ID NO:90.

The present invention provides a multi-specific antibody molecule as defined above, wherein the binding domain specific for human TNF-alpha comprises 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 for CDRL1, SEQ ID NO:89 for CDRL2 and SEQ ID NO:90 for CDRL3.

The present invention provides a multi-specific antibody molecule as defined above, wherein the binding domain specific for human TNF-alpha comprises 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:136 for CDRL1, SEQ ID NO: 89, SEQ ID NO:137, SEQ ID NO: 138 or SEQ ID NO: 139 for CDRL2 and SEQ ID NO:90 for CDRL3.

The present invention provides a multi-specific antibody molecule as defined above, wherein the binding domain specific for human TNF-alpha comprises a heavy chain variable domain comprising the sequence given in SEQ ID NO:92 or SEQ ID NO:96; a sequence having at least 80% identity or similarity to the sequence given in SEQ ID NO:92 or SEQ ID NO:96; or a sequence having at least 80% identity or similarity to the sequence given in SEQ ID NO: 92 or SEQ ID NO:96 wherein CDRH1, CDRH2 and CDRH2 have the sequences given in SEQ ID NO:85 for CDRH1, SEQ ID NO:86 for CDRH2 and SEQ ID NO:87 for CDRH3.

In one example the binding domain specific for human TNF-alpha is humanised. In a humanised antibody of the present invention, the acceptor heavy and light chains do not necessarily need to be derived from the same antibody and may, if desired, comprise composite chains having framework regions derived from different chains.

In one example, one such suitable framework region for the heavy chain of the TNF-alpha binding domain of the present invention is derived from the human VH3 1-3 3-21 JH4 acceptor framework.

Accordingly, in one example the binding domain specific for TNF-alpha comprises a heavy chain variable domain comprising one or more CDRs selected from the sequence given in SEQ ID NO: 85 for CDR-H1, the sequence given in SEQ ID NO: 86 for CDR-H2 and the sequence given in SEQ ID NO: 87 for CDRH3, wherein the heavy chain framework region is derived from the human acceptor framework VH3 1-3 3-21 JH4.

In a humanised antibody of the present invention, the framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

Thus in one embodiment 1, 2, 3, 4, 5, 6, 7, 8, 9 or 10 residues in the heavy and/or light chain framework are replaced with an alternative amino acid residue.

Accordingly, in one example the binding domain specific for TNF-alpha comprises a heavy chain variable domain, wherein at least one of the residues at positions 24, 48, 49 71, 73, 78 and 93 of the variable domain of the heavy chain (Kabat numbering) are donor residues, see for example the sequence given in SEQ ID NO: 92 or SEQ ID NO:96.

In one embodiment residue 24 of the heavy chain variable domain is replaced with an alternative amino acid, for example threonine.

In one embodiment residue 48 of the heavy chain variable domain is replaced with an alternative amino acid, for example isoleucine.

In one embodiment residue 49 of the heavy chain variable domain is replaced with an alternative amino acid, for example glycine.

In one embodiment residue 71 of the heavy chain variable domain is replaced with an alternative amino acid, for example valine.

In one embodiment residue 73 of the heavy chain variable domain is replaced with an alternative amino acid, for example lysine.

In one embodiment residue 78 of the heavy chain variable domain is replaced with an alternative amino acid, for example alanine.

In one embodiment residue 93 of the heavy chain variable domain is replaced with an alternative amino acid, for example threonine.

In one embodiment residue 48 is isoleucine, residue 49 is glycine, 71 is valine, 73 is lysine, 78 is alanine and residue 93 is threonine in the humanised anti-TNF alpha heavy chain variable domain according to the present disclosure.

Accordingly, in one example there is provided a humanised TNF-alpha binding domain, wherein at least the residues at each of positions 48, 49, 71, 73, 78 and 93 of the variable domain of the heavy chain (Kabat numbering) are donor residues, see for example the sequence given in SEQ ID NO:92 or SEQ ID NO:96.

In one example there is provided a humanised TNF-alpha binding domain, wherein at least the residues at each of positions 24, 48, 49, 71, 73, 78 and 93 of the variable domain of the heavy chain (Kabat numbering) are donor residues.

In one example, a suitable framework region for the light chain of the humanised TNF-alpha binding domain of the present invention is derived from the human acceptor framework VK1 2-1(U) A20 JK2.

Accordingly, in one example the binding domain specific for TNF-alpha comprises a light chain variable domain comprising the sequence given in SEQ ID NO: 88 or 136 for CDR-L1, the sequence given in SEQ ID NO: 89, 137, 138 or 139 for CDR-L2 and the sequence given in SEQ ID NO: 90 for CDRL3, wherein the light chain framework region is derived from the human acceptor framework VK1 2-1(U) A20 JK2.

In one example the binding domain specific for TNF-alpha comprises a humanised light chain variable domain wherein one or more of the residues at positions 65, 71 and 87 of the variable domain of the light chain (Kabat numbering) are donor residues, see for example the sequence given in SEQ ID NO: 91 or SEQ ID NO:95.

In one example there is provided a humanised TNF-alpha binding domain, wherein at least the residues at each of positions 65, 71 and 87 of the variable domain of the light chain (Kabat numbering) are donor residues, see for example the sequence given in SEQ ID NO: 91 or 95.

In one embodiment residue 65 of the light chain variable domain is replaced with an alternative amino acid, for example threonine.

In one embodiment residue 71 of the light chain variable domain is replaced with an alternative amino acid, for example tyrosine.

In one embodiment residue 87 of the light chain variable domain is replaced with an alternative amino acid, for example phenylalanine.

In one embodiment residue 65 is threonine, residue 71 is tyrosine and residue 87 is phenylalanine in the humanised anti-TNF-alpha light chain variable region according to the present disclosure.

The present invention provides a multi-specific antibody molecule as defined above, wherein the binding domain specific for human TNF-alpha comprises a light chain variable domain comprising the sequence given in SEQ ID NO:91 or SEQ ID NO:95; a sequence having at least 80% identity or similarity to the sequence given in SEQ ID NO:91 or SEQ ID NO:95; or a sequence having at least 80% identity or similarity to the sequence given in SEQ ID NO:91 or SEQ ID NO:95 wherein CDRL1, CDRL2 and CDRL3 have the sequences given in SEQ ID NO:88 for CDRL1, SEQ ID NO:89 for CDRL2 and SEQ ID NO:90 for CDRL3.

The present invention provides a multi-specific antibody molecule as defined above, wherein the binding domain specific for human TNF-alpha comprises a light chain variable domain comprising the sequence given in SEQ ID NO:91 and a heavy chain variable domain comprising the sequence given in SEQ ID NO:92.

The present invention provides a multi-specific antibody molecule as defined above, wherein the binding domain specific for human TNF-alpha comprises a light chain variable domain comprising the sequence given in SEQ ID NO: 147 and a heavy chain variable domain comprising the sequence given in SEQ ID NO:92.

The present invention provides a multi-specific antibody molecule as defined above, wherein the binding domain specific for human TNF-alpha comprises a light chain variable domain comprising the sequence given in SEQ ID NO:95 and a heavy chain variable domain comprising the sequence given in SEQ ID NO:96.

The present invention provides a multi-specific antibody molecule as defined above, wherein the antibody comprises a dsscFv specific for human TNF-alpha and the dsscFv comprises the sequence given in SEQ ID NO:101; a sequence having at least 80% identity or similarity to the sequence given in SEQ ID NO: 101; or a sequence having at least 80% identity or similarity to the sequence given in SEQ ID NO:101 wherein CDRH1, CDRH2 and CDRH3 have the sequences given in SEQ ID NO:85 for CDRH1, SEQ ID NO:86 for CDRH2 and SEQ ID NO:87 for CDRH3 and CDRL1, CDRL2 and CDRL3 have the sequences given in SEQ ID NO:88 for CDRL1, SEQ ID NO:89 for CDRL2 and SEQ ID NO:90 for CDRL3.

The present invention provides a multi-specific antibody molecule as defined above, wherein the antibody comprises a scFv specific for human TNF-alpha and the scFv comprises the sequence given in SEQ ID NO:99; a sequence having at least 80% identity or similarity to the sequence given in SEQ ID NO:99; or a sequence having at least 80% identity or similarity to the sequence given in SEQ ID NO:99 wherein CDRH1, CDRH2 and CDRH3 have the sequences given in SEQ ID NO:85 for CDRH1, SEQ ID NO:86 for CDRH2 and SEQ ID NO:87 for CDRH3 and CDRL1, CDRL2 and CDRL3 have the sequences given in SEQ ID NO:88 for CDRL1, SEQ ID NO:89 for CDRL2 and SEQ ID NO:90 for CDRL3.

In the aspect of the present invention, wherein the antibody molecule comprises or consists of the polypeptide chains of formula (I) and formula (II), as defined above, the antibody molecule may comprise 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 or 136 for CDRL1, SEQ ID NO: 89 or 137 or 138 or 139 for CDRL2 and SEQ ID NO:90 for CDRL3, are in the position V_{H1}/V_{L1} in the antibody molecule. In one embodiment the 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 or 136 for CDRL1, SEQ ID NO: 89 or 137 or 138 or 139 for CDRL2 and SEQ ID NO:90 for CDRL3 are in the position V₁ in the constructs of the present disclosure. In one embodiment the 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 or 136 for CDRL1, SEQ ID NO: 89 or 137 or 138 or 139 for CDRL2 and SEQ ID NO:90 for CDRL3 are in the position V₂ in the constructs of the present disclosure. In one embodiment the 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 or 136 for CDRL1, SEQ ID NO: 89 or 137 or 138 or 139 for CDRL2 and SEQ ID NO:90 for CDRL3 are in the position V₁ and V₂ in the constructs of the present disclosure. In one embodiment the 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 or 136 for CDRL1, SEQ ID NO: 89 or 137 or 138 or 139 for CDRL2 and SEQ ID NO:90 for CDRL3 are in the position V_{H1}/V_{L1} and V₁ in the constructs of the present disclosure. In one embodiment the 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 or 136 for CDRL1, SEQ ID NO: 89 or 137 or 138 or 139 for CDRL2 and SEQ ID NO:90 for CDRL3 are in the position V_{H1}/V_{L1} and V₂ in the constructs of the present disclosure.

In one embodiment the TNF-alpha binding site in an antibody molecule comprising the polypeptide chains of formula (I) and formula (II) of the present disclosure comprises a heavy variable domain selected from SEQ ID NO: 92 and SEQ ID NO: 96 and a light chain variable domain selected from SEQ ID NO: 91 and SEQ ID NO: 95, in particular SEQ ID NO: 92 and 91 or SEQ ID NO: 96 and 95 for the heavy and light chain respectively. In one embodiment these domains are in the position V_{H1}/V_{L1} in the constructs of the present disclosure. In one embodiment these variable domains are in the position V₁. In one embodiment these variable domains are in the position V₂. In one embodiment these variable domains are in the position V₁ and V₂. In one embodiment these variable domains are in the position V_{H1}/V_{L1} and V₁ in the constructs of the present disclosure. In one embodiment these variable domains are in the position V_{H1}/V_{L1} and V₂ in the constructs of the present disclosure. When the variable domains are in two locations in the constructs of the present disclosure the same pair of variable domains may be in each location or two different pairs of variable domains may be employed.

In one embodiment the TNF-alpha binding site in an antibody molecule comprising the polypeptide chains of formula (I) and formula (II) of the present disclosure comprises a dsscFv specific for human TNF-alpha and the dsscFv comprises the sequence given in SEQ ID NO: 101. In one embodiment the dsscFv comprising the sequence given in SEQ ID NO:101 is in the position V₁ and/or V₂. In one embodiment the dsscFv comprising the sequence given in SEQ ID NO:101 is in the position V₁. In one embodiment the dsscFv comprising the sequence given in SEQ ID NO:101 is in the position V₂.

The present invention provides a multi-specific antibody molecule as defined above, wherein the binding domain specific for human IL-17A and human IL-17F comprises 3 heavy chain CDRs having the sequence given in SEQ ID NO: 71 for CDRH1, SEQ ID NO: 72 for CDRH2 and SEQ ID NO: 73 for CDRH3. In one embodiment, the binding domain specific for human IL-17A and human IL-17F comprises 3 heavy chain CDRs and the sequence of CDRH1 has at least 60% identity or similarity to the sequence given in SEQ ID NO: 71, the sequence of CDRH2 has at least 60% identity or similarity to the sequence given in SEQ ID NO: 72 and the sequence of CDRH3 has at least 60% identity or similarity to the sequence given in SEQ ID NO: 73.

The present invention provides a multi-specific antibody molecule as defined above, wherein the binding domain specific for human IL-17A and human IL-17F comprises 3 light chain CDRs having the sequence given in SEQ ID NO:74 for CDRL1, SEQ ID NO:75 for CDRL2 and SEQ ID NO:76 for CDRL3. In one embodiment, the binding domain specific for human IL-17A and human IL-17F additionally comprises 3 light chain CDRs and the sequence of CDRL1 has at least 60% identity or similarity to the sequence given in SEQ ID NO: 74, the sequence of CDRL2 has at least 60% identity or similarity to the sequence given in SEQ ID NO: 75 and the sequence of CDRL3 has at least 60% identity or similarity to the sequence given in SEQ ID NO: 76.

The present invention provides a multi-specific antibody molecule as defined above, wherein the binding domain specific for human IL-17A and human IL-17F comprises 3 heavy chain CDRs having the sequence given in SEQ ID NO: 71 for CDRH1, SEQ ID NO: 72 for CDRH2 and SEQ ID NO: 73 for CDRH3 and 3 light chain CDRs having the sequence given in SEQ ID NO:74 for CDRL1, SEQ ID NO:75 for CDRL2 and SEQ ID NO:76 for CDRL3.

The present invention provides a multi-specific antibody molecule as defined above, wherein the binding domain specific for human IL-17A and human IL-17F comprises a heavy chain variable domain comprising the sequence given in SEQ ID NO:78; a sequence having at least 80% identity or similarity to the sequence given in SEQ ID NO:78; or a sequence having at least 80% identity or similarity to the sequence given in SEQ ID NO:78 wherein CDRH1, CDRH2 and CDRH3 have the sequences given in SEQ ID NO: 71 for CDRH1, SEQ ID NO: 72 for CDRH2 and SEQ ID NO: 73 for CDRH3.

The present invention provides a multi-specific antibody molecule as defined above, wherein the binding domain specific for human IL-17A and human IL-17F comprises a light chain variable domain comprising the sequence given in SEQ ID NO:77; a sequence having at least 80% identity or similarity to the sequence given in SEQ ID NO:77; or a sequence having at least 80% identity or similarity to the sequence given in SEQ ID NO:77 wherein CDRL1, CDRL2 and CDRL3 have the sequences given in SEQ ID NO:74 for CDRL1, SEQ ID NO:75 for CDRL2 and SEQ ID NO:76 for CDRL3.

The present invention provides a multi-specific antibody molecule as defined above, wherein the binding domain specific for human IL-17A and human IL-17F comprises a heavy chain variable domain comprising the sequence given in SEQ ID NO:78 and a light chain variable domain comprising the sequence given in SEQ ID NO:77.

The present invention provides a multi-specific antibody molecule as defined above, wherein the binding domain specific for human Il-17A and human IL-17F comprises a heavy chain comprising a heavy chain variable domain and heavy chain constant domain comprising the sequence given in SEQ ID NO:82; a sequence having at least 80% identity or similarity to the sequence given in SEQ ID NO:82; or a sequence having at least 80% identity or similarity to the sequence given in SEQ ID NO:82 wherein CDRH1, CDRH2 and CDRH3 have the sequences given in SEQ ID NO: 71 for CDRH1, SEQ ID NO: 72 for CDRH2 and SEQ ID NO: 73 for CDRH3.

The present invention provides a multi-specific antibody molecule as defined above, wherein the binding domain specific for human Il-17A and human IL-17F comprises a light chain comprising a light chain variable domain and light chain constant domain comprising the sequence given in SEQ ID NO:81; a sequence having at least 80% identity or similarity to the sequence given in SEQ ID NO:81; or a sequence having at least 80% identity or similarity to the sequence given in SEQ ID NO:81 wherein CDRL1, CDRL2 and CDRL3 have the sequences given in SEQ ID NO:74 for CDRL1, SEQ ID NO:75 for CDRL2 and SEQ ID NO:76 for CDRL3.

The present invention provides a multi-specific antibody molecule as defined above, wherein the binding domain specific for human IL-17A and human IL-17F comprises a heavy chain having the sequence given in SEQ ID NO: 82 and a light chain having the sequence given in SEQ ID NO:81.

In the aspect of the present invention, wherein the antibody molecule comprises or consists of the polypeptide chains of formula (I) and formula (II), as defined above, the antibody molecule may comprise 3 heavy chain CDRs having the sequence given in SEQ ID NO:71 for CDRH1, SEQ ID NO:72 for CDRH2 and SEQ ID NO:73 for CDRH3 and 3 light chain CDRs having the sequence given in SEQ ID NO:74 for CDRL1, SEQ ID NO: 75 for CDRL2 and SEQ ID NO:76 for CDRL3, are in the position V_{H1}/V_{L1} in the antibody molecule. In one embodiment the 3 heavy chain CDRs having the sequence given in SEQ ID NO:71 for CDRH1, SEQ ID NO:72 for CDRH2 and SEQ ID NO:73 for CDRH3 and 3 light chain CDRs having the sequence given in SEQ ID NO:74 for CDRL1, SEQ ID NO: 75 for CDRL2 and SEQ ID NO:76 for CDRL3 are in the position V₁ in the constructs of the present disclosure. In one embodiment the 3 heavy chain CDRs having the sequence given in SEQ ID NO:71 for CDRH1, SEQ ID NO:72 for CDRH2 and SEQ ID NO:73 for CDRH3 and 3 light chain CDRs having the sequence given in SEQ ID NO:74 for CDRL1, SEQ ID NO: 75 for CDRL2 and SEQ ID NO:76 for CDRL3 are in the position V₂ in the constructs of the present disclosure. In one embodiment the 3 heavy chain CDRs having the sequence given in SEQ ID NO:71 for CDRH1, SEQ ID NO:72 for CDRH2 and SEQ ID NO:73 for CDRH3 and 3 light chain CDRs having the sequence given in SEQ ID NO:74 for CDRL1, SEQ ID NO: 75 for CDRL2 and SEQ ID NO:76 for CDRL3 are in the position V₁ and V₂ in the constructs of the present disclosure. In one embodiment the 3 heavy chain CDRs having the sequence given in SEQ ID NO:71 for CDRH1, SEQ ID NO:72 for CDRH2 and SEQ ID NO:73 for CDRH3 and 3 light chain CDRs having the sequence given in SEQ ID NO:74 for CDRL1, SEQ ID NO: 75 for CDRL2 and SEQ ID NO:76 for CDRL3 are in the position V_{H1}/V_{L1} and V₁ in the constructs of the present disclosure. In one embodiment the 3 heavy chain CDRs having the sequence given in SEQ ID NO:71 for CDRH1, SEQ ID NO:72 for CDRH2 and SEQ ID NO:73 for CDRH3 and 3 light chain CDRs having the sequence given in SEQ ID NO:74 for CDRL1, SEQ ID NO: 75 for CDRL2 and SEQ ID NO:76 for CDRL3 are in the position V_{H1}/V_{L1} and V₂ in the constructs of the present disclosure.

In one embodiment the IL-17A and IL-17F binding site in an antibody molecule comprising the polypeptide chains of formula (I) and formula (II) of the present disclosure comprises a heavy variable domain given in SEQ ID NO: 78 and a light chain variable domain given in SEQ ID NO: 77. In one embodiment these domains are in the position V_{H1}/V_{L1} in the constructs of the present disclosure. In one embodiment these variable domains are in the position V₁. In one embodiment these variable domains are in the position V₂. In one embodiment these variable domains are in the position V₁ and V₂. In one embodiment these variable domains are in the position V_{H1}/V_{L1} and V₁ in the constructs of the present disclosure. In one embodiment these variable domains are in the position V_{H1}/V_{L1} and V₂ in the constructs of the present disclosure.

In one embodiment the IL-17A and IL-17F binding site in an antibody molecule comprising the polypeptide chains of formula (I) and formula (II) of the present disclosure comprises a heavy chain having the sequence given in SEQ ID NO: 82 in the position V_{H1} and a light chain having the sequence given in SEQ ID NO: 81 in the position V_{L1}.

The present invention provides a multi-specific antibody molecule as defined above, wherein the antibody molecule comprises a binding domain specific for human serum albumin which comprises 3 heavy chain CDRs having the sequence given in SEQ ID NO: 103 for CDRH1, SEQ ID NO: 104 for CDRH2 and SEQ ID NO: 105 for CDRH3. In one embodiment, the binding domain specific for human serum albumin comprises 3 heavy chain CDRs and the sequence of CDRH1 has at least 60% identity or similarity to the sequence given in SEQ ID NO: 103, the sequence of CDRH2 has at least 60% identity or similarity to the sequence given in SEQ ID NO: 104 and the sequence of CDRH3 has at least 60% identity or similarity to the sequence given in SEQ ID NO: 105.

The present invention provides a multi-specific antibody molecule as defined above, wherein the antibody molecule comprises a binding domain specific for human serum albumin which comprises 3 light chain CDRs having the sequence given in SEQ ID NO:106 for CDRL1, SEQ ID NO:107 for CDRL2 and SEQ ID NO:108 for CDRL3. In one embodiment, the binding domain specific for human serum albumin additionally comprises 3 light chain CDRs and the sequence of CDRL1 has at least 60% identity or similarity to the sequence given in SEQ ID NO: 106, the sequence of CDRL2 has at least 60% identity or similarity to the sequence given in SEQ ID NO: 107 and the sequence of CDRL3 has at least 60% identity or similarity to the sequence given in SEQ ID NO: 108.

The present invention provides a multi-specific antibody molecule as defined above, wherein the antibody molecule comprises a binding domain specific for human serum albumin which comprises 3 heavy chain CDRs having the sequence given in SEQ ID NO: 103 for CDRH1, SEQ ID NO: 104 for CDRH2 and SEQ ID NO: 105 for CDRH3 and 3 light chain CDRs having the sequence given in SEQ ID NO:106 for CDRL1, SEQ ID NO:107 for CDRL2 and SEQ ID NO:108 for CDRL3.

The present invention also provides a multi-specific antibody binding molecule as defined above, wherein the binding domain specific for human serum albumin comprises a heavy chain variable domain comprising the sequence given in SEQ ID NO: 110 or SEQ ID NO: 114; a sequence having at least 80% identity or similarity to the sequence given in SEQ ID NO:110 or SEQ ID NO:114; or having at least 80% identity or similarity to the sequence given in SEQ ID NO:110 or SEQ ID NO: 114 wherein CDRH1, CDRH2 and CDRH3 have the sequences given in SEQ ID NO: 103 for CDRH1, SEQ ID NO: 104 for CDRH2 and SEQ ID NO: 105 for CDRH3.

The present invention provides a multi-specific antibody molecule as defined above, wherein the binding domain specific for human serum albumin comprises a light chain variable domain comprising the sequence given in SEQ ID NO: 109 or SEQ ID NO: 113 a sequence having at least 80% identity or similarity to the sequence given in SEQ ID NO: 109 or SEQ ID NO: 113; or a sequence having at least 80% identity or similarity to the sequence given in SEQ ID NO:109 or SEQ ID NO:113 wherein CDRL1, CDRL2 and CDRL3 have sequences given in SEQ ID NO:106 for CDRL1, SEQ ID NO:107 for CDRL2 and SEQ ID NO:108 for CDRL3.

The present invention also provides a multi-specific antibody binding molecule as defined above, wherein the binding domain specific for human serum albumin comprises a heavy chain variable domain comprising the sequence given in SEQ ID NO:110 and a light chain variable domain comprising the sequence given in SEQ ID NO: 109.

The present invention also provides a multi-specific antibody binding molecule as defined above, wherein the binding domain specific for human serum albumin comprises a heavy chain variable domain comprising the sequence given in SEQ ID NO:114 and a light chain variable domain comprising the sequence given in SEQ ID NO:113.

The present invention also provides a multi-specific antibody binding molecule as defined above, wherein the antibody comprises a dsscFv specific for human serum albumin and the dsscFv comprises the sequence given in SEQ ID NO:119; a sequence having at least 80% identity or similarity to the sequence given in SEQ ID NO: 119; or a sequence having at least 80% identity or similarity to the sequence of SEQ ID NO:119 wherein CDRH1, CDRH2 and CDRH3 have the sequences given in SEQ ID NO: 103 for CDRH1, SEQ ID NO: 104 for CDRH2 and SEQ ID NO: 105 for CDRH3 and CDRL1, CDRL2 and CDRL3 have the sequences given in SEQ ID NO:106 for CDRL1, SEQ ID NO:107 for CDRL2 and SEQ ID NO:108 for CDRL3.

The present invention also provides a multi-specific antibody binding molecule as defined above, wherein the antibody comprises a scFv specific for human serum albumin and the scFv comprises the sequence given in SEQ ID NO:117; a sequence having at least 80% identity or similarity to the sequence given in SEQ ID NO: 117; or a sequence having at least 80% identity or similarity to the sequence given in SEQ ID NO:117 wherein CDRH1, CDRH2 and CDRH3 have the sequences given in SEQ ID NO: 103 for CDRH1, SEQ ID NO: 104 for CDRH2 and SEQ ID NO: 105 for CDRH3 and CDRL1, CDRL2 and CDRL3 have the sequences given in SEQ ID NO:106 for CDRL1, SEQ ID NO:107 for CDRL2 and SEQ ID NO:108 for CDRL3.

In the aspect of the present invention, wherein the antibody molecule comprises or consists of the polypeptide chains of formula (I) and formula (II), as defined above, the antibody molecule may comprise 3 heavy chain CDRs having the sequence given in SEQ ID NO:103 for CDRH1, SEQ ID NO:104 for CDRH2 and SEQ ID NO:105 for CDRH3 and 3 light chain CDRs having the sequence given in SEQ ID NO:106 for CDRL1, SEQ ID NO: 107 for CDRL2 and SEQ ID NO:108 for CDRL3, are in the position V_{H1}/V_{L1} in the antibody molecule. In one embodiment the 3 heavy chain CDRs having the sequence given in SEQ ID NO:103 for CDRH1, SEQ ID NO:104 for CDRH2 and SEQ ID NO:105 for CDRH3 and 3 light chain CDRs having the sequence given in SEQ ID NO:106 for CDRL1, SEQ ID NO: 107 for CDRL2 and SEQ ID NO:108 for CDRL3 are in the position V₁ in the constructs of the present disclosure. In one embodiment the 3 heavy chain CDRs having the sequence given in SEQ ID NO:103 for CDRH1, SEQ ID NO:104 for CDRH2 and SEQ ID NO:105 for CDRH3 and 3 light chain CDRs having the sequence given in SEQ ID NO:106 for CDRL1, SEQ ID NO: 107 for CDRL2 and SEQ ID NO:108 for CDRL3 are in the position V₂ in the constructs of the present disclosure. In one embodiment the 3 heavy chain CDRs having the sequence given in SEQ ID NO:103 for CDRH1, SEQ ID NO:104 for CDRH2 and SEQ ID NO:105 for CDRH3 and 3 light chain CDRs having the sequence given in SEQ ID NO:106 for CDRL1, SEQ ID NO: 107 for CDRL2 and SEQ ID NO:108 for CDRL3 are in the position V₁ and V₂ in the constructs of the present disclosure. In one embodiment the 3 heavy chain CDRs having the sequence given in SEQ ID NO:103 for CDRH1, SEQ ID NO:104 for CDRH2 and SEQ ID NO:105 for CDRH3 and 3 light chain CDRs having the sequence given in SEQ ID NO:106 for CDRL1, SEQ ID NO: 107 for CDRL2 and SEQ ID NO:108 for CDRL3 are in the position V_{H1}/V_{L1} and V₁ in the constructs of the present disclosure. In one embodiment the 3 heavy chain CDRs having the sequence given in SEQ ID NO:103 for CDRH1, SEQ ID NO:104 for CDRH2 and SEQ ID NO:105 for CDRH3 and 3 light chain CDRs having the sequence given in SEQ ID NO:106 for CDRL1, SEQ ID NO: 107 for CDRL2 and SEQ ID NO:108 for CDRL3 are in the position V_{H1}/V_{L1} and V₂ in the constructs of the present disclosure.

In one embodiment the human serum albumin binding site in an antibody molecule comprising the polypeptide chains of formula (I) and formula (II) of the present disclosure comprises a heavy variable domain selected from SEQ ID NO: 110 and SEQ ID NO: 114 and a light chain variable domain selected from SEQ ID NO: 109 and SEQ ID NO: 113, in particular SEQ ID NO: 110 and 109 or SEQ ID NO: 114 and 113 for the heavy and light chain respectively. In one embodiment these domains are in the position V_{H1}/V_{L1} in the constructs of the present disclosure. In one embodiment these variable domains are in the position V₁. In one embodiment these variable domains are in the position V₂. In one embodiment these variable domains are in the position V₁ and V₂. In one embodiment these variable domains are in the position V_{H1}/V_{L1} and V₁ in the constructs of the present disclosure. In one embodiment these variable domains are in the position V_{H1}/V_{L1} and V₂ in the constructs of the present disclosure. When the variable domains are in two locations in the constructs of the present disclosure the same pair of variable domains may be in each location or two different pairs of variable domains may be employed.

In one embodiment the human serum albumin binding site in an antibody molecule comprising the polypeptide chains of formula (I) and formula (II) of the present disclosure comprises a dsscFv specific for human serum albumin and the dsscFv comprises the sequence given in SEQ ID NO:119. In one embodiment the dsscFv comprising the sequence given in SEQ ID NO:119 is in the position V₁ and/or V₂. In one embodiment the dsscFv comprising the sequence given in SEQ ID NO:119 is in the position V₁. In one embodiment the dsscFv comprising the sequence given in SEQ ID NO: 119 is in the position V₂.

In one aspect of the present invention, the multi-specific antibody molecule as defined above is provided as a dimer comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} represents a heavy chain variable domain comprising 3 heavy chain CDRs having the sequence given in SEQ ID NO: 71 for CDRH1, SEQ ID NO: 72 for CDRH2 and SEQ ID NO: 73 for CDRH3;
CH₁ represents a domain of a heavy chain constant region, for example domain 1 thereof;
X represents a bond or linker, for example having the sequence given in SEQ ID NO: 2;
Y represents a bond or linker, for example having the sequence given in SEQ ID NO: 2;
V₁ represents a dsFv, a sdAb, a scFv or a dsscFv comprising 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 for CDRL1, SEQ ID NO:89 for CDRL2 and SEQ ID NO:90 for CDRL3;
V_{L1} represents a light chain variable domain comprising 3 light chain CDRs having the sequence given in SEQ ID NO:74 for CDRL1, SEQ ID NO:75 for CDRL2 and SEQ ID NO:76 for CDRL3;
C_{L} represents a domain from a light chain constant region, such as Ckappa;
V₂ represents dsFv, a sdAb, a scFv or a dsscFv comprising 3 heavy chain CDRs having the sequence given in SEQ ID NO: 103 for CDRH1, SEQ ID NO: 104 for CDRH2 and SEQ ID NO: 105 for CDRH3 and 3 light chain CDRs having the sequence given in SEQ ID NO:106 for CDRL1, SEQ ID NO:107 for CDRL2 and SEQ ID NO:108 for CDRL3.

In one aspect of the present invention, the multi-specific antibody molecule as defined above is provided as a dimer comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} represents a heavy chain variable domain comprising 3 heavy chain CDRs having the sequence given in SEQ ID NO: 71 for CDRH1, SEQ ID NO: 72 for CDRH2 and SEQ ID NO: 73 for CDRH3;
CH₁ represents a domain of a heavy chain constant region, for example domain 1 thereof;
X represents a bond or linker, for example having the sequence given in SEQ ID NO: 2;
Y represents a bond or linker, for example having the sequence given in SEQ ID NO: 2;
V₁ represents a dsFv, a sdAb, a scFv or a dsscFv comprising 3 heavy chain CDRs having the sequence given in SEQ ID NO: 103 for CDRH1, SEQ ID NO: 104 for CDRH2 and SEQ ID NO: 105 for CDRH3 and 3 light chain CDRs having the sequence given in SEQ ID NO:106 for CDRL1, SEQ ID NO:107 for CDRL2 and SEQ ID NO:108 for CDRL3;
V_{L1} represents a light chain variable domain comprising 3 light chain CDRs having the sequence given in SEQ ID NO:74 for CDRL1, SEQ ID NO:75 for CDRL2 and SEQ ID NO:76 for CDRL3;
C_{L} represents a domain from a light chain constant region, such as Ckappa;
V₂ represents dsFv, a sdAb, a scFv or a dsscFv comprising 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 for CDRL1, SEQ ID NO:89 for CDRL2 and SEQ ID NO:90 for CDRL3.

In one aspect there is provided a multi-specific antibody molecule, comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} represents a heavy chain variable domain having the sequence given in SEQ ID NO: 78;
CH₁ represents a domain of a heavy chain constant region, for example domain 1 thereof;
X represents a linker, for example having the sequence given in SEQ ID NO: 2;
Y represents a linker, for example having the sequence given in SEQ ID NO: 2;
V₁ represents a dsscFv comprising a heavy chain variable domain having the sequence given in SEQ ID NO: 96 and a light chain variable domain having the sequence given in SEQ ID NO: 95;
V_{L1} represents a light chain variable domain having the sequence given in SEQ ID NO: 77;
C_{L} represents a domain from a light chain constant region, such as Ckappa;
V₂ represents a dsscFv comprising a heavy chain variable domain having the sequence given in SEQ ID NO: 114 and a light chain variable domain having the sequence given in SEQ ID NO: 113.

In one aspect there is provided a multi-specific antibody molecule, comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} represents a heavy chain variable domain having the sequence given in SEQ ID NO: 78;
CH₁ represents a domain of a heavy chain constant region, for example domain 1 thereof;
X represents a linker, for example having the sequence given in SEQ ID NO: 2;
Y represents a linker, for example having the sequence given in SEQ ID NO: 2;
V₁ represents a dsscFv comprising a heavy chain variable domain having the sequence given in SEQ ID NO: 114 and a light chain variable domain having the sequence given in SEQ ID NO: 113;
V_{L1} represents a light chain variable domain having the sequence given in SEQ ID NO: 77;
C_{L} represents a domain from a light chain constant region, such as Ckappa;
V₂ represents a dsscFv comprising a heavy chain variable domain having the sequence given in SEQ ID NO: 96 and a light chain variable domain having the sequence given in SEQ ID NO: 95.

In one aspect there is provided a multi-specific antibody molecule, comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} and CH₁ represent a heavy chain variable domain and heavy chain constant domain having the sequence given in SEQ ID NO: 82;
X represents a linker, for example having the sequence given in SEQ ID NO: 2;
Y represents a linker, for example having the sequence given in SEQ ID NO: 2;
V₁ represents a dsscFv comprising a heavy chain variable domain having the sequence given in SEQ ID NO: 96 and a light chain variable domain having the sequence given in SEQ ID NO: 95;
V_{L1} and C_{L} represent a light chain variable domain and light chain constant domain having the sequence given in SEQ ID NO: 81;
V₂ represents a dsscFv comprising a heavy chain variable domain having the sequence given in SEQ ID NO: 114 and a light chain variable domain having the sequence given in SEQ ID NO: 113.

In one aspect there is provided a multi-specific antibody molecule, comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} and CH₁ represent a heavy chain variable domain and heavy chain constant domain having the sequence given in SEQ ID NO: 82;
X represents a linker, for example having the sequence given in SEQ ID NO: 2;
Y represents a linker, for example having the sequence given in SEQ ID NO: 2;
V₁ represents a dsscFv comprising a heavy chain variable domain having the sequence given in SEQ ID NO: 114 and a light chain variable domain having the sequence given in SEQ ID NO: 113;
V_{L1} and C_{L} represent a light chain variable domain and light chain constant domain having the sequence given in SEQ ID NO: 81;
V₂ represents a dsscFv comprising a heavy chain variable domain having the sequence given in SEQ ID NO: 96 and a light chain variable domain having the sequence given in SEQ ID NO: 95.

In one aspect there is provided a multi-specific antibody molecule, comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} and CH₁ represent a heavy chain variable domain and heavy chain constant domain having the sequence given in SEQ ID NO: 82;
X represents a linker, for example having the sequence given in SEQ ID NO: 2;
Y represents a linker, for example having the sequence given in SEQ ID NO: 2;
V₁ represents a dsscFv comprising the sequence given in SEQ ID NO: 101;
V_{L1} and C_{L} represent a light chain variable domain and light chain constant domain having the sequence given in SEQ ID NO: 81;
V₂ represents a dsscFv comprising the sequence given in SEQ ID NO: 119.

In one aspect there is provided a multi-specific antibody molecule, comprising or consisting of:

V_{H1}-CH₁-X-V₁; and a) a polypeptide chain of formula (I):

V_{L1}-C_{L}-Y-V₂; b) a polypeptide chain of formula (II):
wherein:
V_{H1} and CH₁ represent a heavy chain variable domain and heavy chain constant domain having the sequence given in SEQ ID NO: 82;
X represents a linker, for example having the sequence given in SEQ ID NO: 2;
Y represents a linker, for example having the sequence given in SEQ ID NO: 2;
V₁ represents a dsscFv comprising the sequence given in SEQ ID NO: 119;
V_{L1} and C_{L} represent a light chain variable domain and light chain constant domain having the sequence given in SEQ ID NO: 81;
V₂ represents a dsscFv comprising the sequence given in SEQ ID NO: 101.

The present invention provides a tri-specific antibody molecule comprising a binding domain specific to human TNF-alpha, a binding domain specific to human IL-17A and human IL-17F and a binding domain specific to human serum albumin, wherein the antibody molecule comprises or consists of:
a) a first polypeptide comprising or consisting of:
   i. the sequence given in SEQ ID NO:125;
   ii. a sequence having at least 80% identity or similarity to the sequence given in SEQ ID NO: 125; or
   iii. a sequence having at least 80% identity or similarity to the sequence given in SEQ ID NO: 125 wherein the sequence comprises 3 heavy chain CDRs having the sequence given in SEQ ID NO: 71 for CDRH1, SEQ ID NO: 72 for CDRH2 and SEQ ID NO: 73 for CDRH3, 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 for CDRL1, SEQ ID NO:89 for CDRL2 and SEQ ID NO:90 for CDRL3; and
b) a second polypeptide comprising or consisting of:
   i. the sequence given in SEQ ID NO:131;
   ii. a sequence having at least 80% identity or similarity to the sequence given in SEQ ID NO:131; or
   iii. a sequence having at least 80% identity or similarity to the sequence given in SEQ ID NO: 131 wherein the sequence comprises 3 light chain CDRs having the sequence given in SEQ ID NO:74 for CDRL1, SEQ ID NO:75 for CDRL2 and SEQ ID NO:76 for CDRL3, 3 heavy chain CDRs having the sequence given in SEQ ID NO: 103 for CDRH1, SEQ ID NO: 104 for CDRH2 and SEQ ID NO: 105 for CDRH3 and 3 light chain CDRs having the sequence given in SEQ ID NO: 106 for CDRL1, SEQ ID NO: 107 for CDRL2 and SEQ ID NO: 108 for CDRL3.

In one embodiment, the antibody molecule comprises or consists of a first polypeptide having the sequence given in SEQ ID NO: 125; and a second polypeptide having the sequence given in SEQ ID NO:131.

The present invention also provides a tri-specific antibody molecule comprising a binding domain specific to human TNF-alpha, a binding domain specific to human IL-17A and human IL-17F and a binding domain specific to human serum albumin, and comprises or consists of:
a) a first polypeptide comprising or consisting of:
   i. the sequence given in SEQ ID NO:127;
   ii. a sequence having at least 80% identity or similarity to the sequence given in SEQ ID NO: 127; or
   iii. a sequence having at least 80% identity or similarity to the sequence given in SEQ ID NO: 127 wherein the sequence comprises 3 heavy chain CDRs having the sequence given in SEQ ID NO: 71 for CDRH1, SEQ ID NO: 72 for CDRH2 and SEQ ID NO: 73 for CDRH3, 3 heavy chain CDRs having the sequence given in SEQ ID NO:85 for CDRH1, SEQ ID NO:86 for CDRH2 and SEQ ID NO:87 for CDRH3 and 3 light chain CDRs having the sequence given in SEQ ID NO:88 for CDRL1, SEQ ID NO:89 for CDRL2 and SEQ ID NO:90 for CDRL3; and;
b) a second polypeptide comprising or consisting of:
   i. the sequence given in SEQ ID NO:131;
   ii. a sequence having at least 80% identity or similarity to the sequence given in SEQ ID NO:131; or
   iii. a sequence having at least 80% identity or similarity to the sequence given in SEQ ID NO:131 wherein the sequence comprises 3 light chain CDRs having the sequence given in SEQ ID NO:74 for CDRL1, SEQ ID NO:75 for CDRL2 and SEQ ID NO:76 for CDRL3, 3 heavy chain CDRs having the sequence given in SEQ ID NO: 103 for CDRH1, SEQ ID NO: 104 for CDRH2 and SEQ ID NO: 105 for CDRH3 and 3 light chain CDRs having the sequence given in SEQ ID NO: 106 for CDRL1, SEQ ID NO: 107 for CDRL2 and SEQ ID NO: 108 for CDRL3.

In one embodiment, the antibody molecule comprises or consists of a first polypeptide having the sequence given in SEQ ID NO: 127 and a second polypeptide having the sequence given in SEQ ID NO:131.

The present invention also provides a tri-specific antibody molecule comprising a binding domain specific to human TNF-alpha, a binding domain specific to human IL-17A and human IL-17F and a binding domain specific to human serum albumin, and comprises or consists of a first polypeptide having the sequence given in SEQ ID NO: 121; and a second polypeptide having the sequence given in SEQ ID NO: 129.

The present invention also provides a tri-specific antibody molecule comprising a binding domain specific to human TNF-alpha, a binding domain specific to human IL-17A and human IL-17F and a binding domain specific to human serum albumin, and comprises or consists of a first polypeptide having the sequence given in SEQ ID NO: 123; and a second polypeptide having the sequence given in SEQ ID NO: 129.

The present invention also provides a tri-specific antibody molecule comprising a binding domain specific to human TNF-alpha, a binding domain specific to human IL-17A and human IL-17F and a binding domain specific to human serum albumin, which comprises or consists of a first polypeptide encoded by the polynucleotide sequence given in SEQ ID NO:126 and a second polypeptide encoded by the polynucleotide sequence given in SEQ ID NO: 132.

The present invention also provides a tri-specific antibody molecule comprising a binding domain specific to human TNF-alpha, a binding domain specific to human IL-17A and human IL-17F and a binding domain specific to human serum albumin, which comprises or consists of a first polypeptide encoded by the polynucleotide sequence given in SEQ ID NO:144 and a second polypeptide encoded by the polynucleotide sequence given in SEQ ID NO:146.

The present invention also provides a tri-specific antibody molecule comprising a binding domain specific to human TNF-alpha, a binding domain specific to human IL-17A and human IL-17F and a binding domain specific to human serum albumin, which comprises or consists of a first polypeptide encoded by the polynucleotide sequence given in SEQ ID NO:143 and a second polypeptide encoded by the polynucleotide sequence given in SEQ ID NO:145.

In one embodiment the tri-specific antibody molecule according to the present invention comprises or consists of a first polypeptide encoded by a polynucleotide sequence selected from SEQ ID NO: 132, SEQ ID NO:146 or SEQ ID NO:145 and a second polypeptide encoded by a polynucleotide sequence selected from SEQ ID NO: 126, SEQ ID NO: 144 or SEQ ID NO: 143. The tri-specific antibody molecules as defined above, are preferably capable of neutralising the biological activity of human TNF-alpha, human IL-17A and human IL-17F.

The present disclosure also provides sequences which are 80%, 90%, 91%, 92%, 93% 94%, 95% 96%, 97%, 98% or 99% similar to a sequence disclosed herein.
“Identity”, as used herein, indicates that at any particular position in the aligned sequences, the amino acid residue is identical between the sequences.
“Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656).
Antibodies for use in the multispecific constructs of the present invention may be generated by any suitable method known in the art.

Antibodies generated against an antigen polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.
Monoclonal antibodies may be prepared by any method known in the art such as the hybridoma technique (Kohler & Milstein, 1975, Nature, 256:495-497), the trioma technique, the human B-cell hybridoma technique (Kozbor et al 1983, Immunology Today, 4:72) and the EBV-hybridoma technique (Cole et al., Monoclonal Antibodies and Cancer Therapy, pp 77-96, Alan R Liss, Inc., 1985).
Antibodies may also be generated using single lymphocyte antibody methods by cloning and expressing immunoglobulin variable region cDNAs generated from single lymphocytes selected for the production of specific antibodies by, for example, the methods described by Babcook, J. et al 1996, Proc. Natl. Acad. Sci. USA 93(15):7843-78481; WO92/02551; WO2004/051268 and WO2004/106377.
The antibodies for use in the present disclosure can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO90/02809; WO91/10737; WO92/01047; WO92/18619; WO93/11236; WO95/15982; WO95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; 5,969,108, and WO20011/30305.
In one embodiment the multi-specific molecules according to the disclosure are humanised.
Humanised (which include CDR-grafted antibodies) as employed herein refers to molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived.
As used herein, the term “humanised antibody molecule” refers to an antibody molecule wherein the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.
When the CDRs or specificity determining residues are grafted, any appropriate acceptor variable region framework sequence may be used having regard to the class/type of the donor antibody from which the CDRs are derived, including mouse, primate and human framework regions. Suitably, the humanised antibody according to the present invention has a variable domain comprising human acceptor framework regions as well as one or more of the CDRs provided herein.
Examples of human frameworks which can be used in the present disclosure are KOL, NEWM, REI, EU, TUR, TEI, LAY and POM (Kabat et al supra). For example, KOL and NEWM can be used for the heavy chain, REI can be used for the light chain and EU, LAY and POM can be used for both the heavy chain and the light chain. Alternatively, human germline sequences may be used; these are available at: http://www.imgt.org/ or http://www2.mrc-lmb.cam.ac.uk/vbase/list2.Ph.
In a humanised antibody molecule of the present disclosure, the acceptor heavy and light chains do not necessarily need to be derived from the same antibody and may, if desired, comprise composite chains having framework regions derived from different chains.
The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al 1998, Nature, 332, 323-324). Such changes should be kept to the minimum necessary to recover the affinity of the donor antibody. A protocol for selecting residues in the acceptor framework regions which may need to be changed is set forth in WO91/09967.

Derivatives of frameworks may have 1, 2, 3 or 4 amino acids replaced with an alternative amino acid, for example with a donor residue.

Donor residues are residues from the donor antibody, i.e. the antibody from which the CDRs were originally derived. Donor residues may be replaced by a suitable residue derived from a human receptor framework (acceptor residues).
In one embodiment the multi-specific antibodies of the present disclosure are fully human, in particular one or more of the variable domains are fully human.
Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP 0438474 and EP0463151.
In one embodiment the multi-specific antibody molecules of the present disclosure are processed to provide improved affinity for a target antigen or antigens. Such variants can be obtained by a number of affinity maturation protocols including mutating the CDRs (Yang et al J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al Nature, 391, 288-291, 1998). Vaughan et al (supra) discusses these methods of affinity maturation.
Improved affinity as employed herein in this context refers to an improvement over the starting molecule.
If desired a multispecific antibody construct for use in the present disclosure may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al Controlled Drug Delivery, 2nd Ed., Robinson et al eds., 1987, pp. 623-53; Thorpe et al 1982, Immunol. Rev., 62:119-58 and Dubowchik et al 1999, Pharmacology and Therapeutics, 83, 67-123). Particular chemical procedures include, for example, those described in WO93/06231, WO92/22583, WO89/00195, WO89/01476 and WO03031581. Alternatively, where the effector molecule is a protein or polypeptide the linkage may be achieved using recombinant DNA procedures, for example as described in WO86/01533 and EP0392745.
The term “effector molecule” as used herein includes, for example, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.
Other effector molecules may include chelated radionuclides such as 111In and 90Y, Lu177, Bismuth213, Californium252, Iridium192 and Tungsten188/Rhenium188; or drugs such as but not limited to, alkylphosphocholines, topoisomerase I inhibitors, taxoids and suramin.
Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.
Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 11 In and 99Tc.
In another embodiment the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.
Specific optional substituents which may be present on the above-mentioned synthetic polymers include one or more hydroxy, methyl or methoxy groups.
Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one embodiment antibodies for use in the present disclosure are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one embodiment the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, Ala., USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, a F(ab′)₂, Fab or Fab′ in the molecule is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one embodiment PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido)propionamido]ethyl amide of N,N′-bis(methoxypoly(ethylene glycol) MW 20,000) modified lysine, also known as PEG2MAL40K (obtainable from Nektar, formerly Shearwater).

Alternative sources of PEG linkers include NOF who supply GL2-400MA2 (wherein m in the structure below is 5) and GL2-400MA (where m is 2) and n is approximately 450:

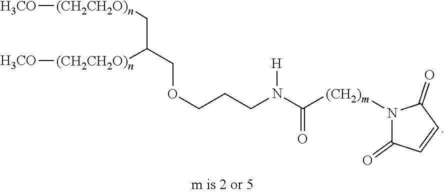

That is to say each PEG is about 20,000 Da.

Further alternative PEG effector molecules of the following type:

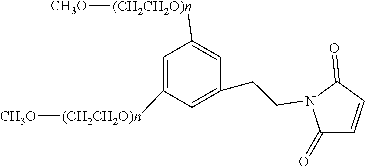

are available from Dr Reddy, NOF and Jenkem.

In one embodiment there is provided an antibody molecule which is PEGylated (for example with a PEG described herein), attached through a cysteine amino acid residue at or about amino acid 226 in the chain, for example amino acid 226 of the heavy chain (by sequential numbering).

In one embodiment there is provided a polynucleotide sequence encoding a molecule of the present disclosure, such as a DNA sequence.

In one embodiment there is provided a polynucleotide sequence encoding one or more, such as two or more, or three or more polypeptide components of a molecule of the present disclosure, for example:

V_{H1}-CH₁-X-V₁; and a) a polypeptide chain of formula (I):

V_{L1}-C_{L}-Y-V₂; b) a polypeptide chain of formula (II):
wherein:
V_{H1} represents a heavy chain variable domain;
CH₁ represents a domain of a heavy chain constant region, for example domain 1 thereof;
X represents a bond or linker;
Y represents a bond or linker;
V₁ represents a dsFv, a sdAb, a scFv or a dsscFv;
V_{L1} represents a light chain variable domain;
C_{L} represents a domain from a light chain constant region, such as Ckappa;
V₂ represents dsFv, a sdAb, a scFV or a dsscFv.

In one embodiment the polynucleotide, such as the DNA is comprised in a vector.

The skilled person will appreciate that when V₁ and/or V₂ represents a dsFv, the multi-specific antibody will comprise a third polypeptide encoding the corresponding free V_{H} or V_{L} domain which is not attached to X or Y. Accordingly the multispecific protein of the present invention may be encoded by one or more, two or more or three or more polynucleotides and these may be incorporated into one or more vectors.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding a multispecific protein of the present invention. Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma, NSO myeloma cells and SP2 cells, COS cells or hybridoma cells.

The present disclosure also provides a process for the production of a multispecific protein according to the present disclosure comprising culturing a host cell containing a vector of the present invention under conditions suitable for leading to expression of protein from DNA encoding the multispecific protein of the present invention, and isolating the multispecific protein.

For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides. In one example the cell line may be transfected with two vectors, each encoding a polypeptide chain of an antibody molecule of the present invention. Where V₁ and/or V₂ are a dsFv the cell line may be transfected with three vectors, each encoding a polypeptide chain of a multispecific protein of the invention.

In one embodiment the cell line is transfected with two vectors each one encoding a different polypeptide selected from:

V_{H1}-CH₁-X-V₁; a) a polypeptide chain of formula (I):

V_{L1}-C_{L}-Y-V₂; b) a polypeptide chain of formula (II):
wherein:
V_{H1} represents a heavy chain variable domain;
CH₁ represents a domain of a heavy chain constant region, for example domain 1 thereof;
X represents a bond or linker;
Y represents a bond or linker;
V₁ represents a dsFv, a sdAb, a scFv or a dsscFv;
V_{L1} represents a light chain variable domain;
C_{L} represents domain from a light chain constant region, such as Ckappa;
V₂ represents dsFv, a sdAb, a scFV or a dsscFv.

In one embodiment when V₁ is a dsFv and the V_{H} domain of V₁ is attached to X, the cell line may be transfected with a third vector which encodes the V_{L} domain of V₁.

In one embodiment when V₁ is a dsFv and the V_{L} domain of V₁ is attached to X, the cell line may be transfected with a third vector which encodes the V_{H} domain of V₁.

In one embodiment when V₂ is a dsFv and the V_{H} domain of V₂ is attached to Y, the cell line may be transfected with a third vector which encodes the V_{L} domain of V₂.

In one embodiment when V₂ is a dsFv and the V_{L} domain of V₂ is attached to Y, the cell line may be transfected with a third vector which encodes the V_{H} domain of V₂.

In one embodiment when both V₁ and V₂ are a dsFv and the V_{L} domain of V₂ is attached to Y and the V_{L} domain of V₁ is attached to X, the cell line may be transfected with a third vector which encodes the common V_{H} domain of both V₁ and V₂.

In one embodiment when both V₁ and V₂ are a dsFv and the V_{H} domain of V₂ is attached to Y and the V_{H} domain of V₁ is attached to X, the cell line may be transfected with a third vector which encodes the common V_{L} domain of both V₁ and V₂.

It will be appreciated that the ratio of each vector transfected into the host cell may be varied in order to optimise expression of the multi-specific antibody product. In one embodiment where two vectors are used the ratio of vectors may be 1:1. In one embodiment where three vectors are used the ratio of vectors may be 1:1:1. It will be appreciated that the skilled person is able to find an optimal ratio by routine testing of protein expression levels following transfection. Alternatively or in addition, the levels of expression of each polypeptide chain of the multi-specific construct from each vector may be controlled by using the same or different promoters.

It will be appreciated that two or more or where present, three of the polypeptide components may be encoded by a polynucleotide in a single vector. It will also be appreciated that where two or more, in particular three or more, of the polypeptide components are encoded by a polynucleotide in a single vector the relative expression of each polypeptide component can be varied by utilising different promoters for each polynucleotide encoding a polypeptide component of the present disclosure.

In one embodiment the vector comprises a single polynucleotide sequence encoding two or where present, three, polypeptide chains of the multispecific antibody molecule of the present invention under the control of a single promoter.

In one embodiment the vector comprises a single polynucleotide sequence encoding two, or where present, three, polypeptide chains of the multispecific antibody molecule of the present disclosure wherein each polynucleotide sequence encoding each polypeptide chain is under the control of a different promoter.

The multispecific proteins according to the present disclosure are expressed at good levels from host cells. Thus the properties of the antibodies and/or fragments appear to be optimised and conducive to commercial processing.

Advantageously, the multi-specific antibody molecules of the present disclosure minimise the amount of aggregation seen after purification and maximise the amount of monomer in the formulations of the construct at pharmaceutical concentrations, for example the monomer may be present as 50%, 60%, 70% or 75% or more, such as 80 or 90% or more such as 91, 92, 93, 94, 95, 96, 97, 98 or 99% or more of the total protein. In one example, a purified sample of a multi-specific antibody molecule of the present disclosure remains greater than 98% or 99% monomeric after 28 days storage at 4° C. In one example, a purified sample of a multi-specific antibody molecule of the present disclosure at 5 mg/ml in phosphate buffered saline (PBS) remains greater than 98% monomeric after 28 days storage at 4° C.

The antibody molecules of the present disclosure and compositions comprising the same are useful in the treatment, for example in the treatment and/or prophylaxis of a pathological condition.

The present disclosure also provides a pharmaceutical or diagnostic composition comprising an antibody molecule of the present disclosure in combination with one or more of a pharmaceutically acceptable excipient, diluent or carrier. Accordingly, provided is the use of an antibody of the present disclosure for use in treatment and for the manufacture of a medicament, in particular for an indication disclosed herein.

The composition will usually be supplied as part of a sterile, pharmaceutical composition that will normally include a pharmaceutically acceptable carrier. A pharmaceutical composition of the present disclosure may additionally comprise a pharmaceutically-acceptable adjuvant.

The present disclosure also provides a process for preparation of a pharmaceutical or diagnostic composition comprising adding and mixing the antibody molecule of the present disclosure together with one or more of a pharmaceutically acceptable excipient, diluent or carrier.

The antibody molecule may be the sole active ingredient in the pharmaceutical or diagnostic composition or may be accompanied by other active ingredients including other antibody ingredients, for example anti-IL-1β, anti-T cell, anti-IFNγ or anti-LPS antibodies, or non-antibody ingredients such as xanthines. Other suitable active ingredients include antibodies capable of inducing tolerance, for example, anti-CD3 or anti-CD4 antibodies.

In a further embodiment the antibody, fragment or composition according to the disclosure is employed in combination with a further pharmaceutically active agent.

The pharmaceutical compositions suitably comprise a therapeutically effective amount of the antibody molecule of the invention. The term “therapeutically effective amount” as used herein refers to an amount of a therapeutic agent needed to treat, ameliorate or prevent a targeted disease or condition, or to exhibit a detectable therapeutic or preventative effect. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 50 mg/kg, for example 0.1 mg/kg to 20 mg/kg. Alternatively, the dose may be 1 to 500 mg per day such as 10 to 100, 200, 300 or 400 mg per day. Pharmaceutical compositions may be conveniently presented in unit dose forms containing a predetermined amount of an active agent of the invention.

Compositions may be administered individually to a patient or may be administered in combination (e.g. simultaneously, sequentially or separately) with other agents, drugs or hormones.

The dose at which the antibody molecule of the present disclosure is administered depends on the nature of the condition to be treated, the extent of the inflammation present and on whether the antibody molecule is being used prophylactically or to treat an existing condition.

The frequency of dose will depend on the half-life of the antibody molecule and the duration of its effect. If the antibody molecule has a short half-life (e.g. 2 to 10 hours) it may be necessary to give one or more doses per day. Alternatively, if the antibody molecule has a long half-life (e.g. 2 to 15 days) it may only be necessary to give a dosage once per day, once per week or even once every 1 or 2 months.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

In one embodiment, in formulations according to the present disclosure, the pH of the final formulation is not similar to the value of the isoelectric point of the antibody or fragment, for if the pH of the formulation is 7 then a p of from 8-9 or above may be appropriate. Whilst not wishing to be bound by theory it is thought that this may ultimately provide a final formulation with improved stability, for example the antibody or fragment remains in solution.

The pharmaceutical compositions of this disclosure may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared. Preferably the antibody molecules of the present invention are administered subcutaneously, by inhalation or topically.

Direct delivery of the compositions will generally be accomplished by injection, subcutaneously, intraperitoneally, intravenously or intramuscularly, or delivered to the interstitial space of a tissue. The compositions can also be administered into a specific tissue of interest. Dosage treatment may be a single dose schedule or a multiple dose schedule.

It will be appreciated that the active ingredient in the composition will be an antibody molecule. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Remington's Pharmaceutical Sciences (Mack Publishing Company, N.J. 1991).

In one embodiment the formulation is provided as a formulation for topical administrations including inhalation.

Suitable inhalable preparations include inhalable powders, metering aerosols containing propellant gases or inhalable solutions free from propellant gases (such as nebulisable solutions or suspensions). Inhalable powders according to the disclosure containing the active substance may consist solely of the abovementioned active substances or of a mixture of the above mentioned active substances with physiologically acceptable excipient.

These inhalable powders may include monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these with one another. Mono- or disaccharides are suitably used, the use of lactose or glucose, particularly but not exclusively in the form of their hydrates.

Particles for deposition in the lung require a particle size less than 10 microns, such as 1-9 microns for example from 0.1 to 5 μm, in particular from 1 to 5 μm. The particle size of the active agent (such as the antibody or antibody fragment) is of primary importance.

The propellent gases which can be used to prepare the inhalable aerosols are known in the art. Suitable propellent gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellent gases may be used on their own or in mixtures thereof.

Particularly suitable propellent gases are halogenated alkane derivatives selected from among TG 11, TG 12, TG 134a and TG227. Of the abovementioned halogenated hydrocarbons, TG134a (1,1,1,2-tetrafluoroethane) and TG227 (1,1,1,2,3,3,3-heptafluoropropane) and mixtures thereof are particularly suitable.

The propellent-gas-containing inhalable aerosols may also contain other ingredients such as cosolvents, stabilisers, surface-active agents (surfactants), antioxidants, lubricants and means for adjusting the pH. All these ingredients are known in the art.

The propellant-gas-containing inhalable aerosols according to the invention may contain up to 5% by weight of active substance. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active.

Alternatively topical administrations to the lung may also be by administration of a liquid solution or suspension formulation, for example employing a device such as a nebulizer, for example, a nebulizer connected to a compressor (e.g., the Pari LC-Jet Plus® nebulizer connected to a Pari Master® compressor manufactured by Pari Respiratory Equipment, Inc., Richmond, Va.).

In one embodiment the formulation is provided as discrete ampoules containing a unit dose for delivery by nebulisation.

In one embodiment the antibody is supplied in lyophilised form, for reconstitutions or alternatively as a suspension formulation.

The antibody of the present disclosure can be delivered dispersed in a solvent, e.g., in the form of a solution or a suspension. It can be suspended in an appropriate physiological solution, e.g., physiological saline, a pharmacologically acceptable solvent or a buffered solution. Buffered solutions known in the art may contain 0.05 mg to 0.15 mg disodium edetate, 8.0 mg to 9.0 mg NaCl, 0.15 mg to 0.25 mg polysorbate, 0.25 mg to 0.30 mg anhydrous citric acid, and 0.45 mg to 0.55 mg sodium citrate per 1 ml of water so as to achieve a pH of about 4.0 to 5.0. As mentioned supra a suspension can made, for example, from lyophilised antibody.

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes.

This may include production and sterilization by filtration of the buffered solvent solution used for the formulation, aseptic suspension of the antibody in the sterile buffered solvent solution, and dispensing of the formulation into sterile receptacles by methods familiar to those of ordinary skill in the art.

Nebulisable formulation according to the present disclosure may be provided, for example, as single dose units (e.g., sealed plastic containers or vials) packed in foil envelopes. Each vial contains a unit dose in a volume, e.g., 2 ml, of solvent/solution buffer.

The antibodies of the present disclosure are thought to be suitable for delivery via nebulisation.

It is also envisaged that the antibody of the present invention may be administered by use of gene therapy. In order to achieve this, DNA sequences encoding the heavy and light chains of the antibody molecule under the control of appropriate DNA components are introduced into a patient such that the antibody chains are expressed from the DNA sequences and assembled in situ.

The present invention also provides a multi-specific antibody molecule as defined above for use in therapy.

The present invention also provides a multi-specific antibody molecule as defined above for the control of inflammatory diseases. Preferably, the multi-specific antibody molecule can be used to reduce the inflammatory process or to prevent the inflammatory process.

The present invention also provides the multi-specific antibody molecule of the present invention for use in the treatment or prophylaxis of a pathological disorder that is mediated by TNF-alpha and IL-17A and/or IL-17F or is associated with an increased level of TNF-alpha and IL-17A and/or IL-17F. Preferably, the pathological condition is selected from the group consisting of infections (viral, bacterial, fungal and parasitic), endotoxic shock associtated with infection, arthritis, rheumatoid arthritis, psoriatic arthritis, systemic onset juvenile idiopathic arthritis (JIA), systemic lupus erythematosus (SLE), asthma, chronic obstructive airways disease (COAD), chronic obstructive pulmonary disease (COPD), acute lung injury, pelvic inflammatory disease, Alzheimer's Disease, Crohn's disease, inflammatory bowel disease, irritable bowel syndrome, Ulcerative colitis, Castleman's disease, ankylosing spondylitis and other spondyloarthropathies, dermatomyositis, myocarditis, uveitis, exophthalmos, autoimmune thyroiditis, Peyronie's Disease, coeliac disease, gallbladder disease, Pilonidal disease, peritonitis, psoriasis, atopic dermatitis, vasculitis, surgical adhesions, stroke, autoimmune diabetes, Type I Diabetes, lyme arthritis, meningoencephalitis, immune mediated inflammatory disorders of the central and peripheral nervous system such as multiple sclerosis and Guillain-Barr syndrome, other autoimmune disorders, pancreatitis, trauma (surgery), graft-versus-host disease, transplant rejection, fibrosing disorders including pulmonary fibrosis, liver fibrosis, renal fibrosis, scleroderma or systemic sclerosis, cancer (both solid tumours such as melanomas, hepatoblastomas, sarcomas, squamous cell carcinomas, transitional cell cancers, ovarian cancers and hematologic malignancies and in particular acute myelogenous leukaemia, chronic myelogenous leukemia, chronic lymphatic leukemia, gastric cancer and colon cancer), heart disease including ischaemic diseases such as myocardial infarction as well as atherosclerosis, intravascular coagulation, bone resorption, osteoporosis, periodontitis and hypochlorhydia.

In one embodiment the antibody of the present invention is used in the treatment or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, scleroderma, systemic sclerosis, lung fibrosis, inflammatory bowel diseases, ankylosing spondylitis and other spondyloarthropathies and cancer.

In one embodiment the antibody of the present invention is used in the treatment or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, scleroderma, systemic sclerosis, lung fibrosis, Crohn's disease, ulcerative colitis, ankylosing spondylitis and other spondyloarthropathies and cancer.

In one embodiment the antibody of the present invention is used in the treatment or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, scleroderma, systemic sclerosis, lung fibrosis, Crohn's disease, ulcerative colitis, ankylosing spondylitis and other spondyloarthropathies.

In one embodiment the antibody of the present invention is used in the treatment or prophylaxis of a pathological disorder selected from the group consisting of arthritis, psoriatic arthritis, systemic onset juvenile idiopathic arthritis (JIA), Crohn's disease, inflammatory bowel disease, Ulcerative colitis, ankylosing spondylitis and other spondyloarthropathies, psoriasis, hidradenitis suppurativa, Behcet's disease, rheumatoid arthritis, systemic lupus erythematosus (SLE), asthma, myocarditis, uveitis, atopic dermatitis, vasculitis, immune mediated inflammatory disorders of the central and peripheral nervous system such as multiple sclerosis and Guillain-Barr syndrome, other autoimmune disorders, osteoporosis, bone resorption, gout, sarcoidosis, Sjogren syndrome and pyoderma gangrenosum.

In one embodiment the pathological disorder is rheumatoid arthritis.

In one embodiment the pathological disorder is Crohn's disease.

In one embodiment the pathological disorder is ulcerative colitis.

In one embodiment the pathological disorder is uveitis.

In one example the antibody of the present invention is used in the treatment of an inflammatory or immune related disease. In one example the inflammatory or immune related disease is selected from the group consisting of rheumatoid arthritis, Crohn's disease and ulcerative colitis.

The present invention also provides an antibody molecule according to the present invention for use in the treatment or prophylaxis of pain, particularly pain associated with inflammation.

The present invention further provides the use of an antibody molecule according to the present invention in the manufacture of a medicament for the treatment or prophylaxis of a pathological disorder that is mediated by TNF-alpha and IL-17A and/or IL-17F or associated with an increased level of IL-17A and/or IL-17F. Preferably the pathological disorder is one of the medical indications described herein above. The present invention further provides the use of an antibody molecule according to the present invention in the manufacture of a medicament for the treatment or prophylaxis of pain, particularly pain associated with inflammation.

An antibody molecule of the present invention may be utilised in any therapy where it is desired to reduce the effects of TNF-alpha and IL-17A and/or IL-17F in the human or animal body. TNF-alpha and IL-17A and/or IL-17F may be circulating in the body or may be present in an undesirably high level localised at a particular site in the body, for example a site of inflammation.

An antibody molecule according to the present invention is preferably used for the control of inflammatory disease, autoimmune disease or cancer.

The present invention also provides a method of treating human or animal subjects suffering from or at risk of a disorder mediated by TNF-alpha and IL-17A and/or IL-17F, the method comprising administering to the subject an effective amount of an antibody molecule of the present invention.

An antibody molecule according to the present invention may also be used in diagnosis, for example in the in vivo diagnosis and imaging of disease states involving TNF-alpha and IL-17A and/or IL-17F.

Thus there is provided a multi-specific antibody according to the present disclosure for use in treatment and methods of treatment employing same.

In one embodiment there is provided a process for purifying a multi-specific antibody (in particular an antibody or fragment according to the invention).

In one embodiment there is provided a process for purifying a multi-specific antibody (in particular an antibody or fragment according to the invention) comprising the steps: performing anion exchange chromatography in non-binding mode such that the impurities are retained on the column and the antibody is maintained in the unbound fraction. The step may, for example be performed at a pH about 6-8.

The process may further comprise an initial capture step employing cation exchange chromatography, performed for example at a pH of about 4 to 5.

The process may further comprise of additional chromatography step(s) to ensure product and process related impurities are appropriately resolved from the product stream.

The purification process may also comprise of one or more ultra-filtration steps, such as a concentration and diafiltration step.

Purified form as used supra is intended to refer to at least 90% purity, such as 91, 92, 93, 94, 95, 96, 97, 98, 99% w/w or more pure.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 μg per mg of antibody product or less such as 100 μg per mg or less, in particular 20 μg per mg, as appropriate.

The antibody molecule of the present invention may also be used in diagnosis, for example in the in vivo diagnosis and imaging of disease states.

In one embodiment there is provided a method of selecting a multi-specific protein construct according to the present invention comprising:
a) determining the yield of monomeric multi-specific protein when either V₁ or V₂ is a dsscFv for a given variable region pair
b) determining the yield of monomeric multi-specific protein when whichever of V₁ or V₂ was tested in (a) is a dsFv for the same variable region pair and
c) comparing the yield of monomer obtained in (a) and (b) and selecting the multi-specific protein with the highest monomeric yield.

Typically the “variable region pair” in step (a) and (b) of the method are a V_{H} and V_{L} pair. Generally V_{H} and V_{L} together form an antigen binding domain, V₁ or V₂. The method therefore allows the V_{H} and V_{L} pair to be tested as both a dsscFv and a dsFv in the constructs of the present invention and the most monomeric construct is selected in step (c).

Typically in step (a) and (b) of the method the yield is determined following purification, such as following affinity chromatography. Monomer yield may be determined using any suitable method, such as size exclusion chromatography.

“Comprising” in the context of the present specification is intended to meaning including. Where technically appropriate, embodiments of the invention may be combined. Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present disclosure is further described by way of illustration only in the following examples, which refer to the accompanying Figures, in which:

### BRIEF DESCRIPTION OF THE FIGURES

FIG. 1 to 7 show amino acid and nucleic acid sequences of antibody molecules according to the present invention

FIG. 8 shows the Fab-2xdsscFv and Fab-dsscFv-dsFv formats

FIG. 9 shows the Titration curves for CA2109 mouse Fab (fwk18 FAB) IgG1 (fwk18 IgG)

FIG. 10a shows representative cell survival curve for L929 cells treated with Actinomycin-D/and human TNF-α and TrYbe 18B or the control compound Enbrel.

FIG. 10b shows representative cell survival curve for L929 cells treated with Actinomycin-D/and cynomolgus TNF-α and TrYbe 18B or the control compound Enbrel.

FIG. 11 shows TrYbe 18B inhibits human TNFα-induced neutrophilia in mice.

FIG. 12 shows that TrYbe 18B inhibits combined human TNFα and human IL-17A-induced neutrophilia in mice.

FIG. 13 shows SDS PAGE Analysis of Different Batches of TrYbe 18B.

FIG. 14 shows TrYbe 18B inhibits neutrophil migration in vitro.

FIG. 15 shows serum concentrations of TrYbe18B following IV bolus doses of TrYbe 18B to cynomolgus monkeys.

### EXAMPLES

### Example 1: Isolation of Neutralizing Anti-Human TNF-Alpha Variable Regions

The following immunizations were performed in order to generate material for B cell culture and antibody screening:

5 Sprague Dawley rats were immunised with 3 shots of human TNF-alpha pre-complexed with a small molecule benzimidazole compound, Compound 1 (as described in WO2013/186229 and PCT/EP2015/074527). Sera was generated and tested for binding to human TNF-alpha in an ELISA. Titres were measurable beyond a 100,000 dilution and were therefore considered acceptable for B cell culturing.

B cell cultures were prepared using a method similar to that described by Zubler et al. (1985) and Lightwood et al. (2013). Briefly, splenocytes containing B cells, at a density of approximately 5000 cells per well were cultured in bar-coded 96-well tissue culture plates with 200 al/well RPMI 1640 medium (Gibco BRL) supplemented with 10% FCS (PAA laboratories ltd), 2% HEPES (Sigma Aldrich), 1% L-Glutamine (Gibco BRL), 1% penicillin/streptomycin solution (Gibco BRL), 0.1% β-mercaptoethanol (Gibco BRL), 2-5% activated splenocyte culture supernatant and gamma-irradiated murine thymoma cells (5×10⁴/well) for seven days at 37° C. in an atmosphere of 5% CO₂. Over 70 million B cells were screened during this project.

The presence of human TNF-specific antibodies in B cell culture supernatants was determined using a homogeneous fluorescence-based binding assay using 10 micron superavidin polymeric beads (Bangs laboratories) coated with biotinylated human TNF as a source of target antigen. 10 ul of supernatant was transferred from barcoded 96-well tissue culture plates into barcoded 384-well black-walled assay plates containing 5000 the coated beads using a Matrix Platemate liquid handler. Binding was revealed with a goat anti-rat or mouse IgG Fcγ-specific Cy-5 conjugate (Jackson). Plates were read on an Applied Biosystems 8200 cellular detection system.

Alternatively, ELISA assays were used to identify positive wells. 384-well ELISA plates were coated with 2 ug/ml TNF before 10 ul of B cell culture supernatant was added to the blocked plate. Following incubation for 1 hour plates were washed and binding revealed with a goat anti-rat Fc-specific HRP conjugate (Jackson).

Following primary screening, positive supernatants were consolidated onto 96-well bar-coded master plates using an Aviso Onyx hit-picking robot and B cells in cell culture plates frozen at −80 C. Master plates were then screened in a Biacore assay in order to identify wells containing antibodies of high affinity.

In order to identify antibodies capable of neutralising the biological activity of TNFα, we performed a cell-based TNFα reporter assay using the B cell culture supernatants in master plates. The assay utilised HEK-293-CD40-BLUE cells (Invivogen) which are engineered to secrete alkaline phosphatase in response to a number of stimuli operating through the NFkB pathway including human TNFα. Antibody-containing supernatants were used directly in this assay at a single dilution of 1:2.5. Wells containing high affinity blocking antibodies (sub 100 pM in Biacore and showing >90% inhibition in the reporter assay) were selected for further progression.

To allow recovery of antibody variable region genes from a selection of wells of interest, a deconvolution step had to be performed to enable identification of the antigen-specific B cells in a given well that contained a heterogeneous population of B cells. This was achieved using the Fluorescent foci method. Briefly, Immunoglobulin-secreting B cells from a positive well were mixed with streptavidin beads (New England Biolabs) coated with biotinylated human TNF and a 1:1200 final dilution of a goat anti-rat Fcγ fragment-specific FITC conjugate (Jackson). After static incubation at 37° C. for 1 hour, antigen-specific B cells could be identified due to the presence of a fluorescent halo surrounding that B cell. These individual B cells, identified using an Olympus microscope, were then picked with an Eppendorf micromanipulator and deposited into a PCR tube.

Antibody variable region genes for four different antibodies known as 2102, 2101, 2109 and 2111 were recovered from single cells by reverse transcription (RT)-PCR using heavy and light chain variable region-specific primers. Two rounds of PCR were performed on an Aviso Onyx liquid handling robot, with the nested 2° PCR incorporating restriction sites at the 3′ and 5′ ends allowing cloning of the rat variable region into a mouse γ1 IgG or Fab (VH) or mouse kappa (VL) mammalian expression vector. Heavy and light chain constructs were co-transfected into HEK-293 cells using Fectin 293 (Invitrogen) and recombinant antibody expressed in 48-well plates in a volume of 1 ml. After 5-7 days expression, supernatants were harvested and antibody subject to further screening.

Recombinant rat/mouse chimeric IgG and Fab molecules were screened in the HEK-293-CD40-BLUE reporter assay at a number of concentrations to enable the calculation of EC50 values and determine the maximum percentage inhibition. Fab fragments were tested in order to ensure the antibody was active when monovalent. IgG were also analysed in a Biacore experiment to determine binding affinities for human TNF. The assay format of the Biacore experiment was capture of the mouse IgG by immobilised anti-mouse IgG-Fc then titration of human TNF over the captured surface. BIA (Biamolecular Interaction Analysis) was performed using a Biacore T200 (GE Healthcare). Affinipure F(ab′)2 Fragment goat anti-mouse IgG, Fc fragment specific (Jackson ImmunoResearch) was immobilised on a CM5 Sensor Chip via amine coupling chemistry to a level of ˜5000 response units (RUs). HBS-EP buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of mouse IgG at 0.5 μg/mL was used for capture by the immobilised anti-mouse IgG-Fc. Human TNF, was injected over the captured mouse IgG twice at 20 nM at a flow rate of 30 μL/min. The surface was regenerated by 2×10 μL injection of 40 mM HCl, interspersed by a 5 μL injection of 5 mM NaOH at a flowrate of 10 μL/min. Background subtraction binding curves were analysed using the T200evaluation software (version 1.0) following standard procedures.

TABLE 4

| | EC50 | % | ka | kd | KD |
|---|---|---|---|---|---|
| Antibody | (ng/ml) | inhibition | (1/Ms) | (1/s) | (pM) |
| CA2109 IgG | 0.31 | 100 | 4.70E+06 | 1.19E−04 | 25 |
| CA2109 Fab | 3.8 | 100 | ND | ND | ND |

Neutralisation was determined using the HEK-293-CD40-BLUE reporter assay (Invivogen). EC50 and % neutralisation is shown. Biacore analysis was performed to determine binding kinetics. On rate (ka), off rate (kd) and affinity constant (KD) are shown.

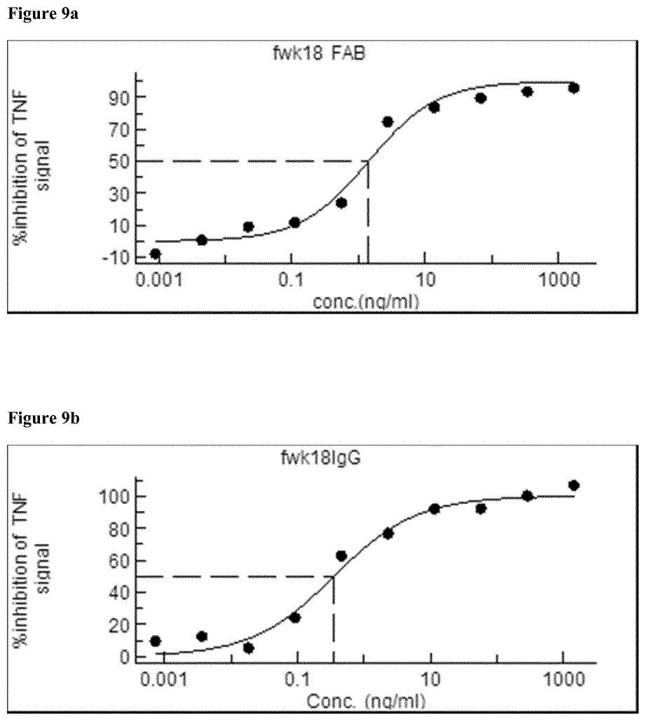

FIG. 9 shows the Titration curves for CA2109 rat/mouse Fab (fwk18 FAB) IgG1 (fwk18 IgG). Data determined using the HEK-293-CD40-BLUE reporter assay (Invivogen) as described above.

All four antibodies (2101, 2109, 2111 and 2102) isolated as described above were screened as rat/human chimeric Fabs for cyno and human TNF cross-reactivity on human TNF (hTNF) and cyno TNF (cTNF) as shown in Table 4a below.

The assay format of the Biacore experiment was capture of the rat/human chimeric Fabs by immobilised anti-human F(ab′) then titration of human or cynomolgus TNF over the captured surface. BIA (Biamolecular Interaction Analysis) was performed using a Biacore T200 (GE Healthcare). Affinipure F(ab′)2 Fragment goat anti-human IgG-F(ab′)2 fragment specific (Jackson ImmunoResearch) was immobilised on a CM5 Sensor Chip via amine coupling chemistry to a level of ˜5000 response units (RUs). HBS-EP buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of rat/human chimeric at 0.5 μg/mL was used for capture by the immobilised human IgG-F(ab′)2. Human or cynomolgus TNF, was injected over the captured rat/human chimeric Fab at (5 nM or 3.125 nM respectively) at a flow rate of 30 μL/min. The surface was regenerated by 2×10 μL injection of 50 mM HCl, interspersed by a 5 μL injection of 5 mM NaOH at a flowrate of 10 μL/min. Background subtraction binding curves were analysed using the T200evaluation software (version 1.0) following standard procedures.

TABLE 4a

| | | | ka | kd | KD | KD |
|---|---|---|---|---|---|---|
| | | | (1/Ms) | (1/s) | (M) | (pM) |
| 02101 | human Fab | hTNF | 4.63E+06 | 1.39E−04 | 2.99E−11 | 30 |
| | chimeric | | | | | |
| 02109 | human Fab | hTNF | 4.09E+06 | 1.43E−04 | 3.49E−11 | 35 |
| | chimeric | | | | | |
| 02111 | human Fab | hTNF | 2.89E+06 | 1.39E−04 | 4.81E−11 | 48 |
| | chimeric | | | | | |
| 02102 | human Fab | hTNF | 2.59E+06 | 2.27E−04 | 8.73E−11 | 87 |
| | chimeric | | | | | |
| 02101 | human Fab | cTNF | 4.54E+06 | 9.72E−05 | 2.14E−11 | 21 |
| | chimeric | | | | | |
| 02109 | human Fab | cTNF | 3.71E+06 | 1.02E−04 | 2.74E−11 | 27 |
| | chimeric | | | | | |
| 02111 | human Fab | cTNF | 2.26E+06 | 2.25E−04 | 9.98E−11 | 100 |
| | chimeric | | | | | |
| 02102 | human Fab | cTNF | 4.17E+05 | 8.64E−03 | 2.07E−08 | 20720 |
| | chimeric | | | | | |

Antibody 2102 was found to have a much lower affinity for cyno TNF. This antibody also lost affinity during humanisation so was not progressed any further.

Based on this work, CA2109 was selected as a lead candidate due to its cyno/human cross reactivity, high affinity (Table 4) and potent neutralisation activity (FIG. 9 and Table 4). This antibody was selected for humanisation based on these properties.

Two other antibodies having similar affinity and neutralisation properties were selected for humanisation in parallel to CA2109, these were 2101 and 2111. However, 2101 failed to retain affinity for TNF upon humanisation so this antibody was not progressed any further. Antibody 2109 and antibody 2111 were both successfully humanised and then converted to a scFv format and screened in an L929 TNF inhibition assay (assay described in Example 6 below) to confirm neutralisation activity (Table 4b).

TABLE 4b

| | IC50 |
|---|---|
| Antibody | (pM) |
| CA2111 Fab | 12.49 |
| CA2111 VH1 HLds scFv | 135.09 |
| CA2111 VH1 LHds scFv | 143.24 |
| CA2109 VH3 scFv | 16.55 |

As can be seen in Table 4b, antibody 2111 did not retain activity in this assay as a scFv so was not suitable for use in a multi-specific TrYbe antibody molecule. Only antibody 2109 retained cyno-human cross reactivity, high affinity and neutralisation capability and thermostability as a humanised scFv. The humanisation of antibody CA2109 is described in more detail below.

### Example 2: Humanization of Anti-TNF-Alpha Antibody CA2109

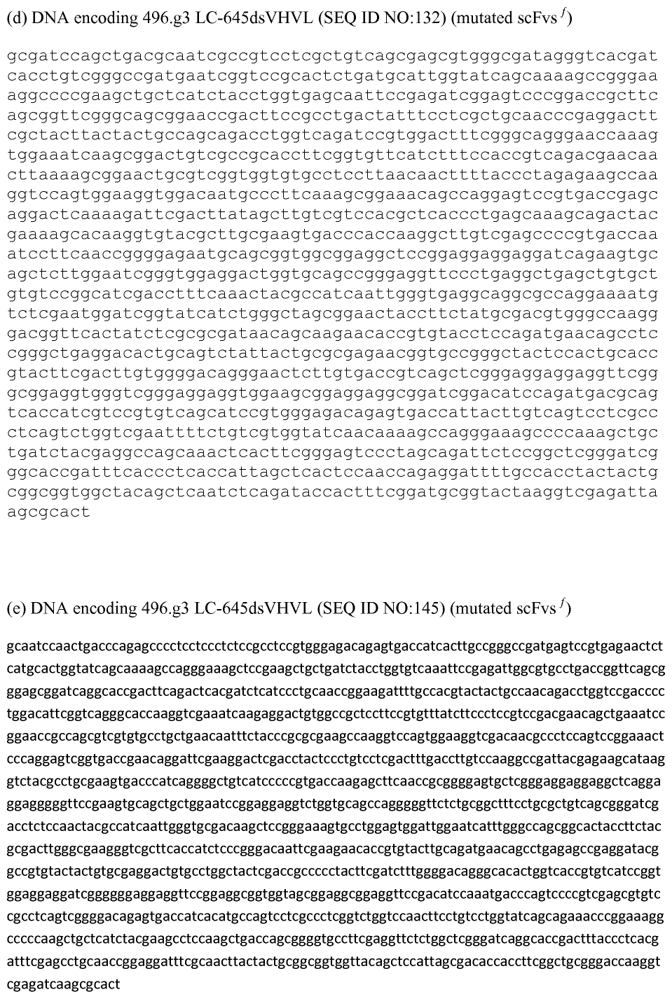

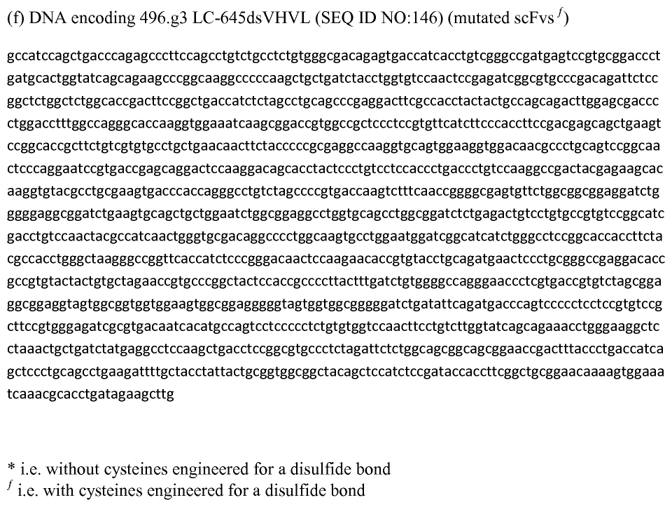

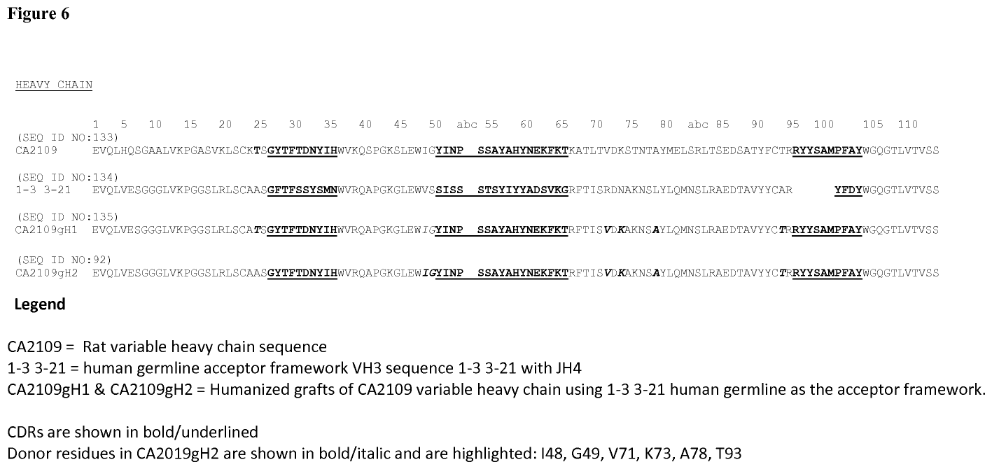

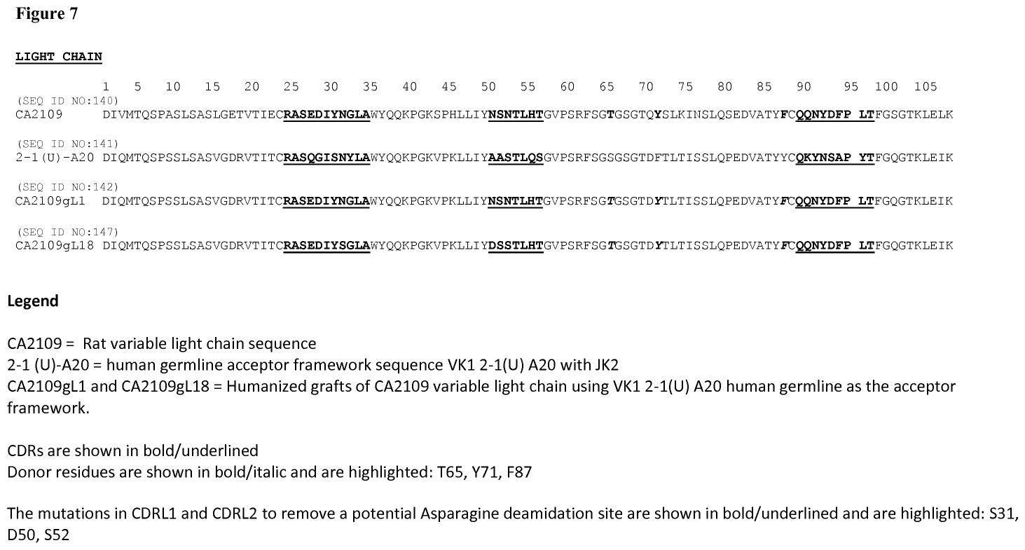

Antibody CA2109 was humanised by grafting the complementarity determining regions (CDR) onto human germline frameworks. Alignments of the rat antibody (donor) sequence with the human germline (acceptor) frameworks are shown in FIGS. 6 and 7, together with the designed humanised sequences.

The light chain germline acceptor sequence chosen was the human VK1 2-1(U) A20 V-region plus JK2 J-region (V BASE, http://vbase.mrc-cpe.cam.ac.uk/). The heavy chain germline acceptor sequence chosen was the human VH3 1-3 3-21 V-region plus JH4 J-region (V BASE, http://vbase.mrc-cpe.cam.ac.uk/). The CDRs grafted from the donor to the acceptor sequence are as defined by Kabat (Kabat et al. 1987), with the exception of CDR-H1 where the combined Chothia/Kabat definition is used.

Genes encoding initial V-region sequences were designed and constructed by an automated synthesis approach by Entelechon GmbH, and modified to generate the grafted versions μL1, μL18, gH1 and gH2 by oligonucleotide directed mutagenesis. The μL18 gene sequence was sub-cloned into the UCB Celltech human light chain expression vector pMhCK delta, which contains DNA encoding the human C-Kappa constant region (Km3 allotype). The gH2 sequence was sub-cloned into the UCB Celltech expression vector pMhg1Fab, which contains DNA encoding human heavy chain gamma-1 CH1 constant region.

In order to retain full activity and maintain high thermostability, donor residues at positions 48 (Isoleucine), 49 (Glycine), 71 (Valine), 73 (Lysine), 78 (Alanine) and 93 (Threonine) of the humanised heavy chain (Kabat numbering) were retained. Similarly, donor residues at positions 65 (Threonine), 71 (Tyrosine) and 87 (Phenylalanine) of the humanised light chain (Kabat numbering) were retained. In addition, 3 deamidation sites in the light chain were removed by mutating Asparagine residues at positions 31, 50 and 52 to Serine, Aspartic acid and Serine, respectively. The final selected variable graft sequences μL18 and gH2 are shown in FIGS. 6 and 7 and SEQ ID NO: 91 and 92. In the following Examples, any reference to antibody 2109 refers to these humanised sequences rather than the original rat parent sequences.

### Example 3: Construction of Fab496.g3-(HC)dsscFv(HL)2109 and Fab496.g3-(LC) dsscFv(HL)645 Plasmids and Expression in Mammalian Cells

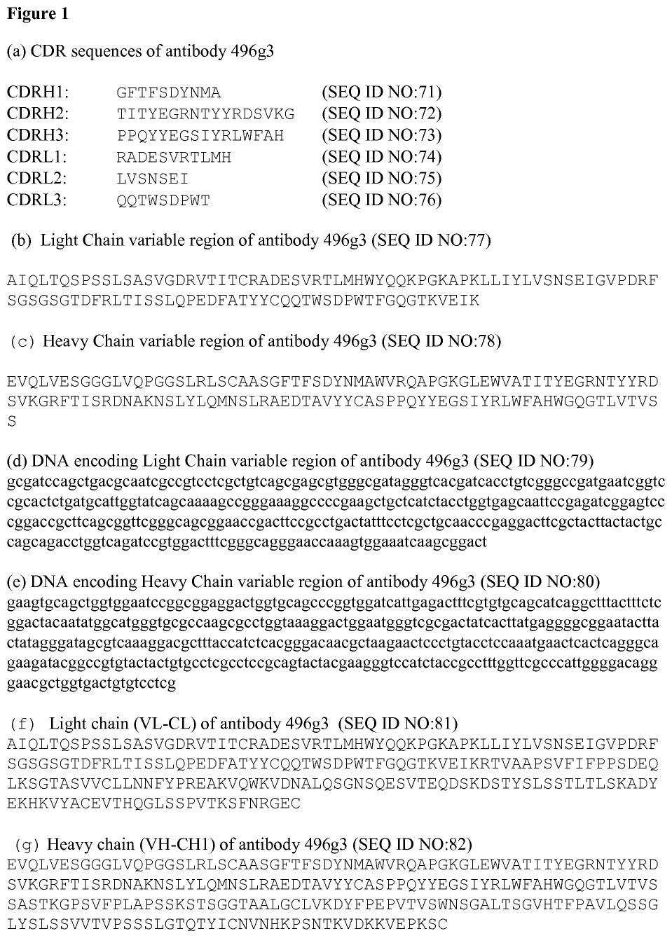

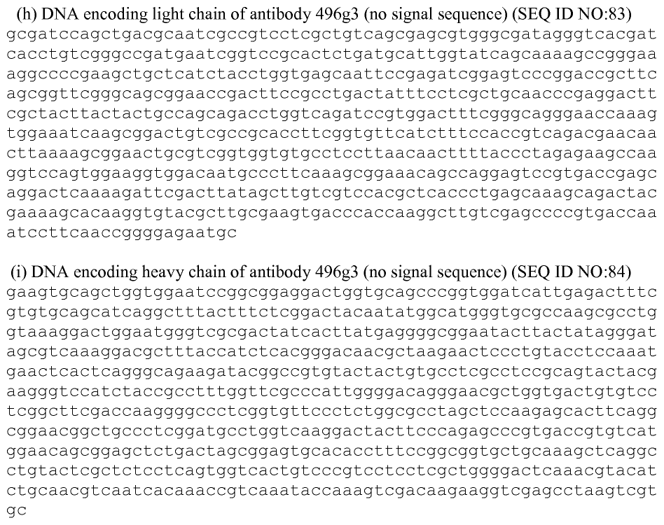

The production of the antibody CA028_00496.g3 (also referred to herein as antibody 496.g3) against human IL-17A and human IL-17F has been previously described in WO2008/047134 and WO2012/095662. The antibody binds human IL-17A, IL-17F and IL-17A/F heterodimer with pM affinity. The amino acid and DNA sequences encoding the CDRs, heavy and light variable regions and light chain and heavy chain of the Fab format of antibody 496.g3 are shown in FIG. 1. The 496.g3 (IL-17A/F binding) Fab constant regions comprised the human C-kappa constant region (K1m3 allotype) and the human gamma-1 CH1 constant region and hinge (G1m17 allotype).

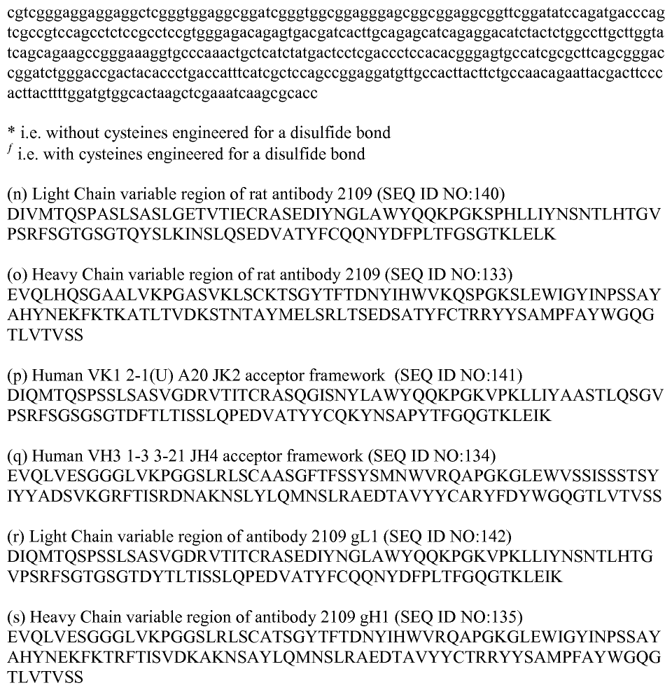

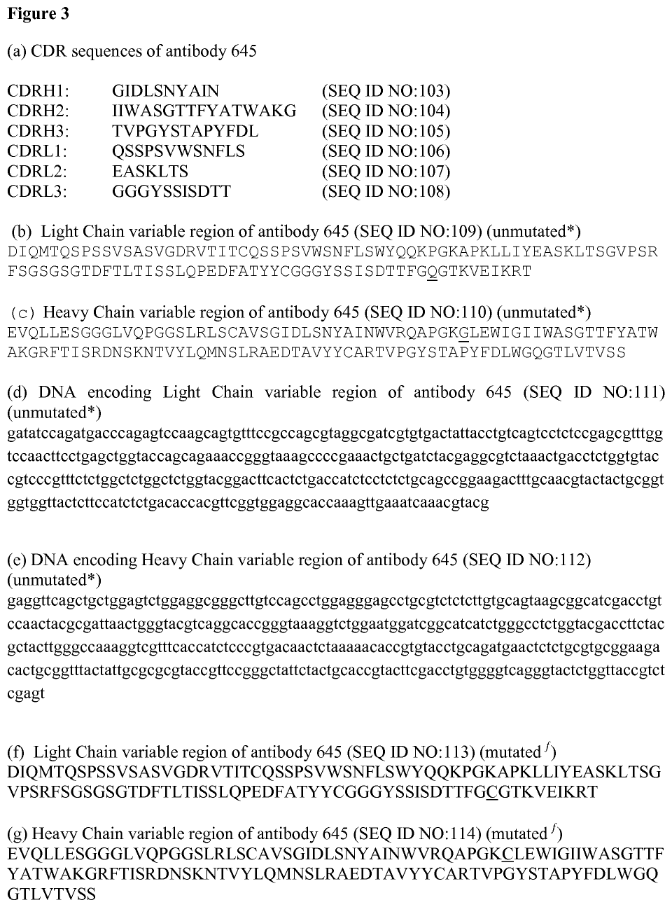

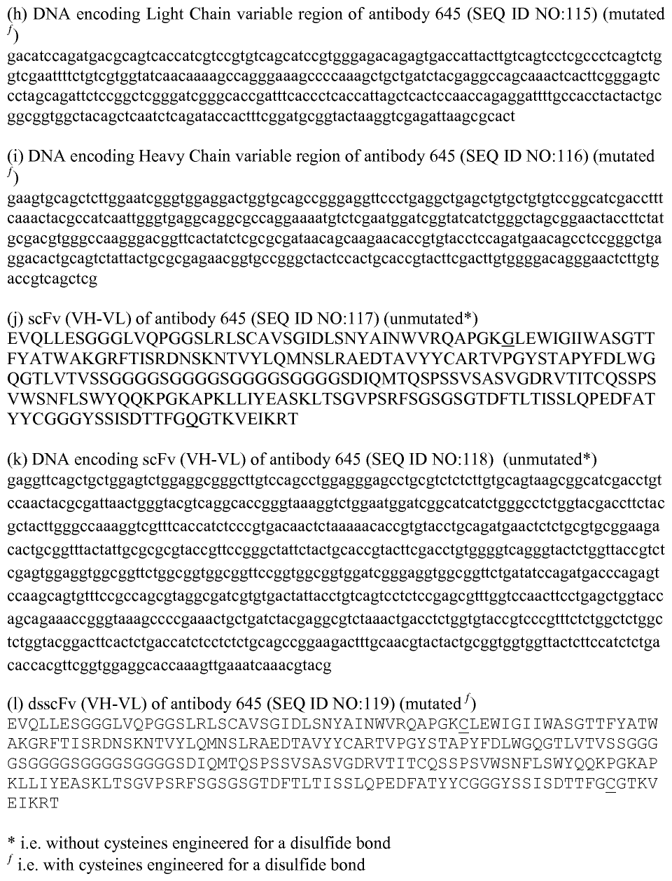

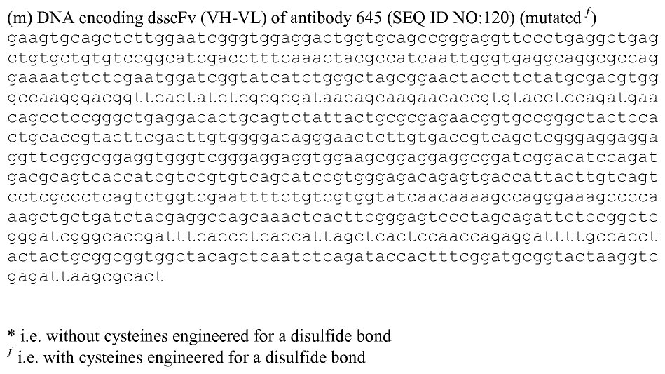

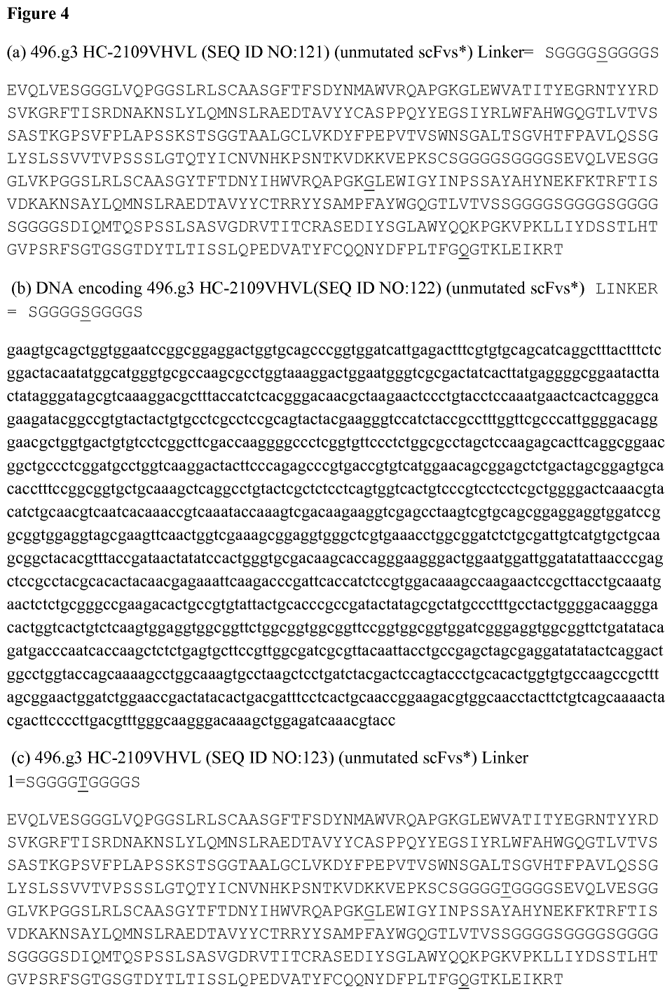

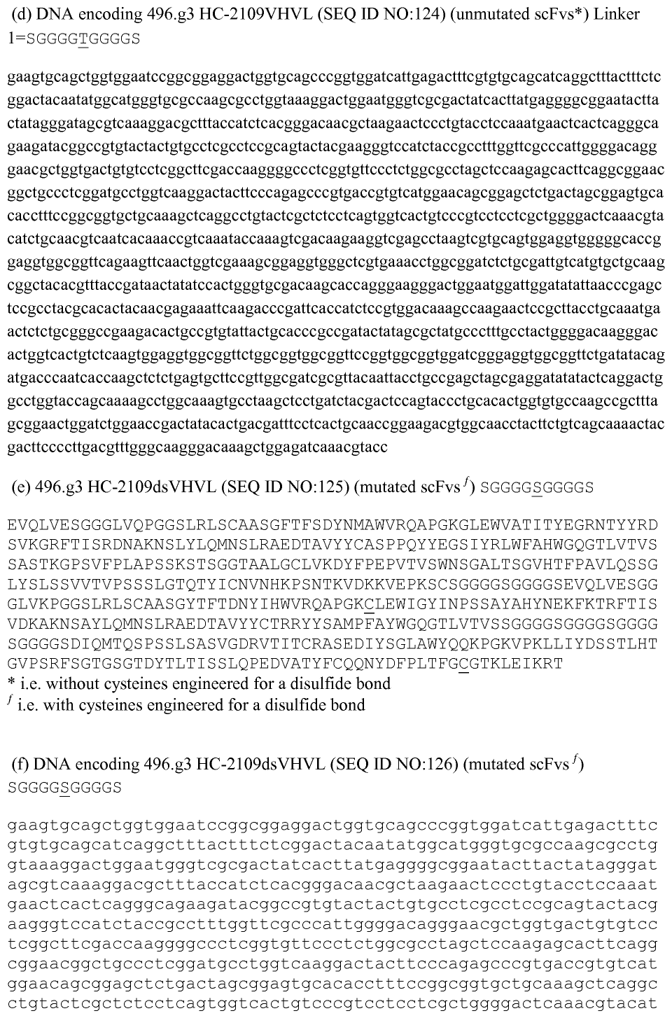

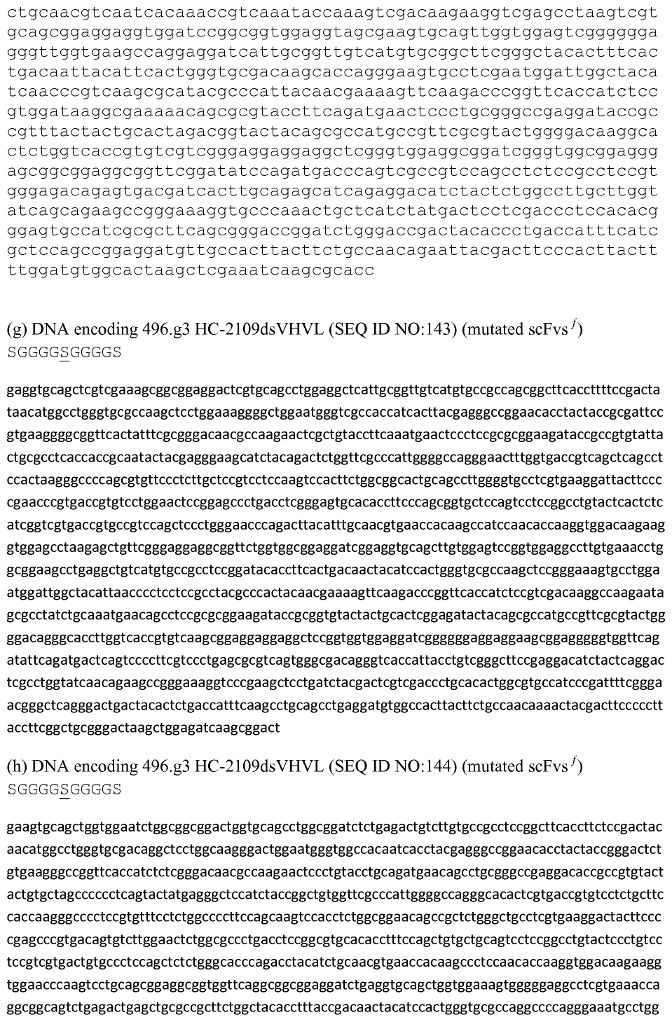

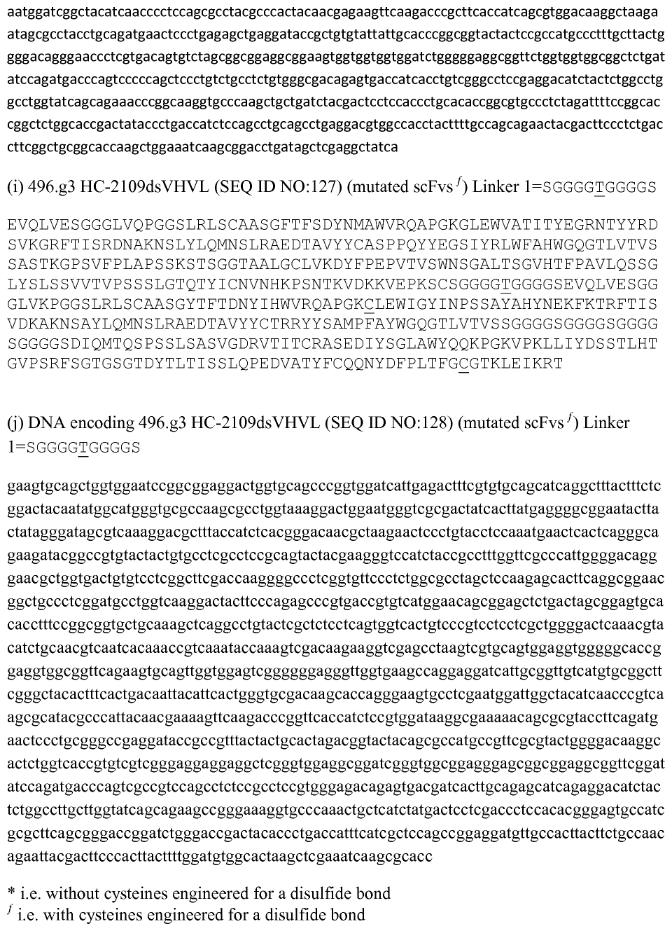

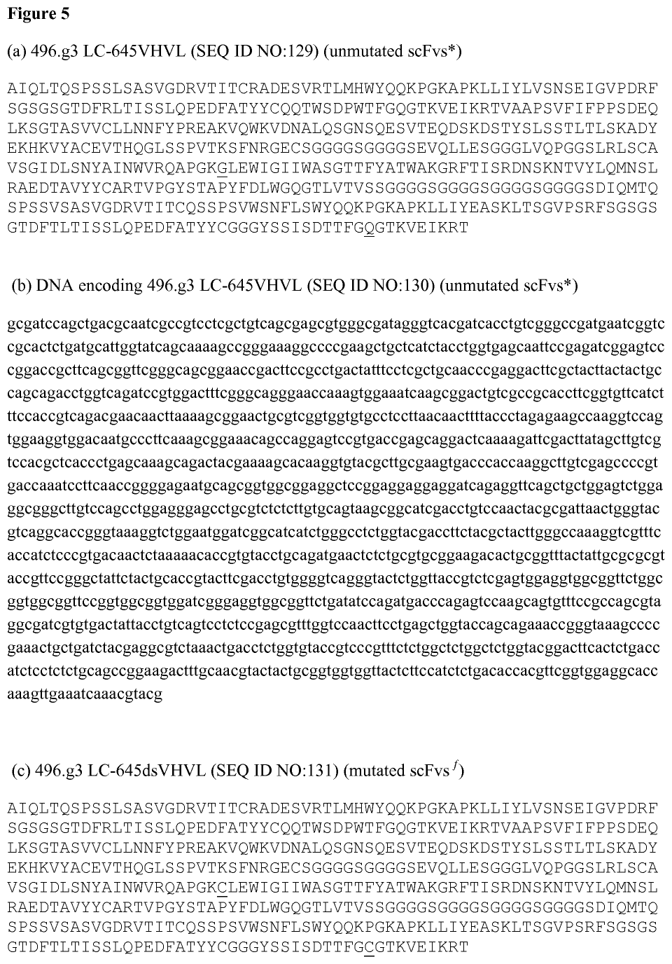

The production of the anti-human albumin antibody 645 has been previously described in WO2013/068571. The amino acid and DNA sequences encoding the CDRs, heavy and light variable regions, scFv and dsscFV formats of antibody 645 are shown in FIG. 3.

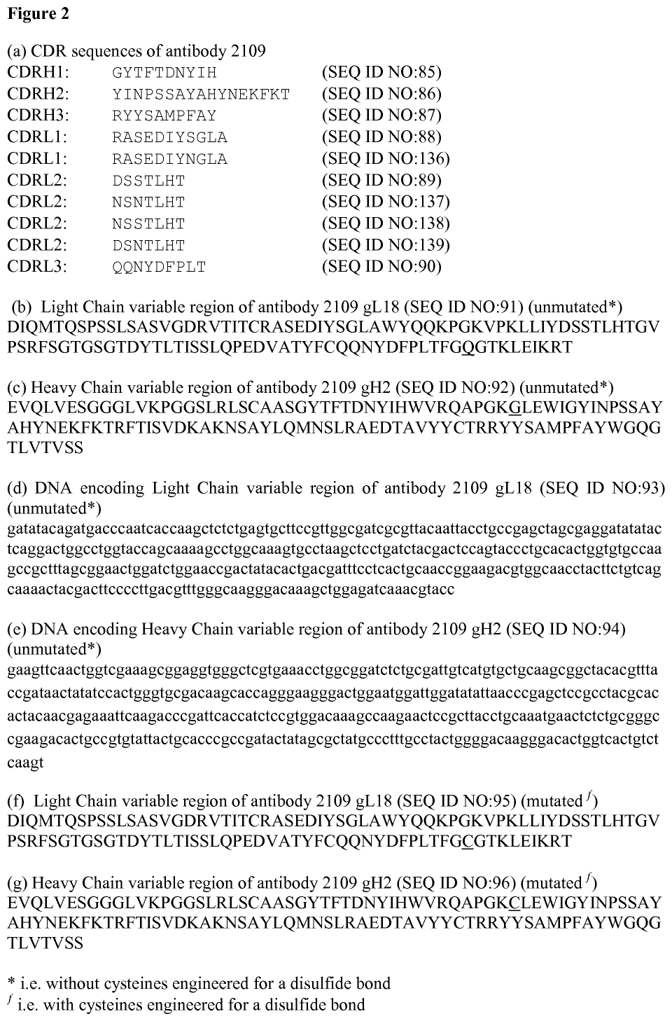

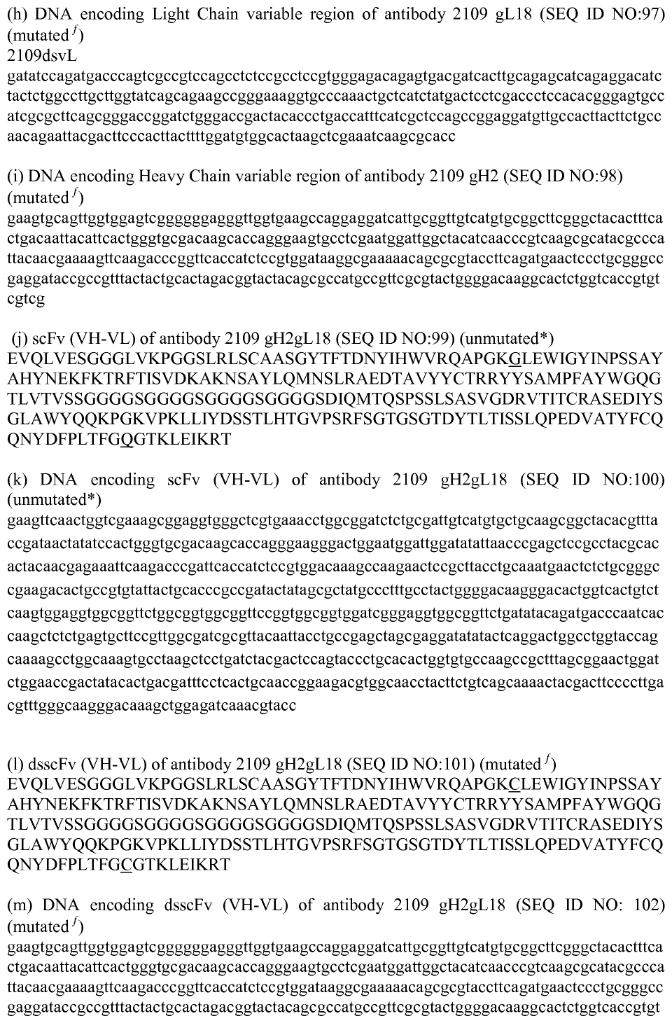

The production of anti-TNF antibody 2109 is described above and the amino acid and DNA sequences encoding the CDRs, heavy and light variable regions, scFv and dsscFV formats of antibody 2109 are shown in FIG. 2.

The dsscFv of antibody 2109 and antibody 645 each contained a stabilising disulphide bond between residues 44 (heavy chain) and 100 (light chain) using the Kabat numbering system.

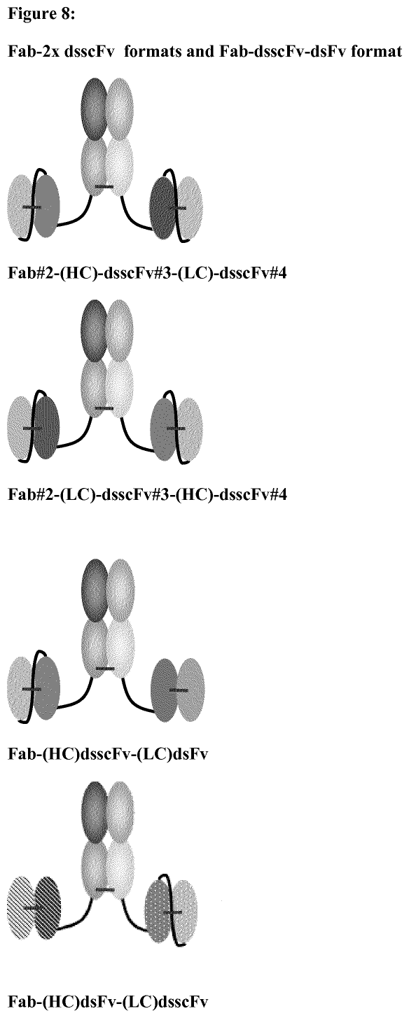

The dsscFv of antibody 645 HL was connected to the Fab cKappa fragment of antibody 496.g3 by an 11 amino acid light chain linker (SGGGGSGGGGS) and the dsscFv of antibody CA2109 HL was connected to the Fab CH1 fragment of antibody 496.g3 by an 11 amino acid heavy chain linker SGGGGTGGGGS [also referred herein as S, 2xG4S] (SEQ ID NO: 2) or SGGGGTGGGGS [also referred to herein as S, G4T, G4S] (SEQ ID NO: 1). The antibody format generated is illustrated in FIG. 8 as 2xdsscFv and known herein as TrYbe.

Transient expression was used to express Fab496.g3-(HC)dsscFv(HL)2109 (SEQ ID NO: 127) (having the linker SGGGGTGGGGS connecting the Fab HC and the dsscFv2109) and Fab496.g3-(LC) dsscFv(HL)645 (SEQ ID NO: 131) (having the linker SGGGGSGGGGS connecting the Fab LC and the dsscFv645). Genes encoding the entire light chain and the heavy chain Fab fragment were restriction cloned from plasmids that were previously optimised at the codon level for expression in E. coli. The Agel-EcoRI gene fragment encoding 645 dsHL scFv was excised from pTrYbe HC #1 and cloned into pED489 to generate a ‘light chain single gene vector’ 496.g3 LC-645 dsHL scFv in the UCB in-house expression vector pNAFL (Dhami, 2012). DNA encoding 2109 dsHL scFv (BspEI-EcoRI fragment) was synthesised by DNA2.0 and cloned into pTrYbe HC #1 (Dhami, 2012) to generate a ‘heavy chain single gene vector’ 496.g3 Fab-2109 dsHL scFv in the UCB in-house expression vector pNAFH (Dhami, 2012). Following transient co-expression of both single gene vectors in HEK293 cells, TrYbe protein comprising Fab496.g3-(HC)dsscFv(HL)2109 having the heavy chain linker SGGGGTGGGGS and Fab496.g3-(LC) dsscFv(HL)645 was purified from the cell culture supernatant. Purified TrYbe displayed a high level of monomer (89%) and high thermal stability (Tm 71° C.), and was thus progressed for stable cell line generation.

Stable expression was used to express Fab496.g3-(HC)dsscFv(HL)2109 (SEQ ID NO:125) (having the linker SGGGGSGGGGS connecting the Fab HC to the dsscFv2109), and Fab496.g3-(LC) dsscFv(HL)645 (SEQ ID NO: 131) (having the linker SGGGGSGGGGS connecting the FabLC to the dsscFv645), which is also referred to herein as antibody molecule Trybe 18B, from a mammalian CHO cell line with productivity levels of approximately 1 g/L and approximately 70% monomer. A downstream purification process was carried out in order to generate purified TrYbe18B.

### Example 4 Biacore Affinity and Simultaneous Binding of Antigen Targets of Fab496.g3-(HC)dsHLscFv2109 (LC)dsHLscFv645 (Trybe 18B)

The antibody molecule comprising Fab496.g3-(HC)dsscFv(HL)2109 (SEQ ID NO:125) (having the linker SGGGGSGGGGS connecting the Fab HC to the dsscFv2109), and Fab496.g3-(LC) dsscFv(HL)645 (SEQ ID NO: 131) (having the linker SGGGGSGGGGS connecting the FabLC to the dsscFv645), which is also referred to herein as antibody molecule Trybe 18B as produced according to the method in Example 3 was tested for affinity against human TNF-alpha, human IL-17A, human IL-17F and human serum albumin according to the method described below:

The assay format was capture of the TrYbe 18B by immobilised anti-human IgG-F(ab′)₂ then titration of Human TNF, Human IL-17A, Human IL-17F and Human Serum Albumin over the captured surface. BIA (Biamolecular Interaction Analysis) was performed using a Biacore T200 (GE Healthcare). Affinipure F(ab′)2 Fragment goat anti-human IgG, F(ab′)2 fragment specific (Jackson ImmunoResearch) was immobilised on a CM5 Sensor Chip via amine coupling chemistry to a capture level of ˜5000 response units (RUs). HBS-EP buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of TrYbe 18B at 0.5 μg/mL was used for capture by the immobilised anti-human IgG-F(ab′)2. Human TNF, Human IL-17A, Human IL-17F and HSA were titrated over the captured TrYbe 18B at various concentrations (5 nM to 0.15625 nM, 5 nM to 0.15625 nM, 10 nM to 0.3125 nM and 100 nM to 3.125 nM respectively) at a flow rate of 30 μL/min. The surface was regenerated by 2×10 μL injection of 50 mM HCl, interspersed by a 5 μL injection of 5 mM NaOH at a flowrate of 10 μL/min. Background subtraction binding curves were analysed using the T200evaluation software (version 1.0) following standard procedures. Kinetic parameters were determined from the fitting algorithm. The results are shown in Table 5.

TABLE 5

| Sample | ka(1/Ms) | kd(1/s) | KD(M) | KD(pM) |
|---|---|---|---|---|
| Human TNF | 5.54E+06 | 6.43E−05 | 1.16E−11 | 11.6 |
| Human IL-17A | 5.52E+06 | 1.00E−05* | 1.81E−12 | 1.81 |
| Human IL-17F | 5.06E+06 | 3.80E−05 | 7.51E−12 | 7.51 |
| Human | 7.75E+04 | 1.35E−04 | 1.74E−09 | 1740 |
| Albumin | | | | |

1.00E−05* Dissociation limited due to limit of the instrument

A further assay was performed to measure the binding of antibody molecule TrYbe 18B to Cynomolgus TNF, Cynomolgus IL-17A, Cynomolgus IL-17F and Cynomolgus Albumin. The assay format was capture of the TrYbe 18B by immobilised anti-human IgG-F(ab′)₂ then titration of Cynomolgus TNF, IL-17A, IL-17F and Albumin over the captured surface. BIA (Biamolecular Interaction Analysis) was performed using a Biacore T200 (GE Healthcare). Affinipure F(ab′)2 Fragment goat anti-human IgG, F(ab′)2 fragment specific (Jackson ImmunoResearch) was immobilised on a CM5 Sensor Chip via amine coupling chemistry to a capture level of ˜5000 response units (RUs). HBS-EP buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of TrYbe 18B at 0.5 μg/mL was used for capture by the immobilised anti-human IgG-F(ab′)2. Cynomolgus TNF, IL-17A, IL-17F and Albumin were titrated over the captured TrYbe 18B at various concentrations (5 nM to 0.15625 nM, 10 nM to 0.3125 nM, 40 nM to 1.25 nM and 100 nM to 3.125 nM respectively) at a flow rate of 30 μL/min. The surface was regenerated by 2×10 μL injection of 50 mM HCl, interspersed by a 5 μL injection of 5 mM NaOH at a flowrate of 10 L/min. Background subtraction binding curves were analysed using the T200evaluation software (version 1.0) following standard procedures. Kinetic parameters were determined from the fitting algorithm. The results are shown in Table 6.

TABLE 6

| Sample | ka(1/Ms) | kd(1/s) | KD(M) | KD(nM) |
|---|---|---|---|---|
| Cynomolgus TNF | 8.90E+06 | 6.38E−05 | 7.16E−12 | 7.16 |
| Cynomolgus IL-17A | 1.67E+06 | 1.00E−05* | 5.99E−12 | 5.99 |
| Cynomolgus IL-17F | 5.82E+05 | 1.67E−04 | 2.88E−10 | 288 |
| Cynomolgus | 5.83E+04 | 1.59E−04 | 2.72E−9 | 2723 |
| Albumin | | | | |

1.00E−05* Dissociation limited due to limit of the instrument

The simultaneous binding of Human IL-17A, Human TNF and Human Serum Albumin to TrYbe 18B was assessed. The TrYbe 18B construct was captured to the sensor chip by immobilised anti-human IgG-F(ab′)₂ then hIL-17A only, hTNF only, HSA only or a mixed solution of hIL-17A, hTNF and HSA were titrated separately over the captured TrYbe 18B.

BIA (Biamolecular Interaction Analysis) was performed using a Biacore T200 (GE Healthcare). The TrYbe 18B construct was captured to the sensor chip surface as stated in the method above for Biacore kinetics for TrYbe 18B binding human TNF, IL-17A, IL-17F and HSA. 100 nM HSA, 20 nM hIL-17A, 20 nM hTNF or a mixed solution with final concentration of 100 nM HSA, 20 nM hIL-17A, 20 nM hTNF were titrated separately over the captured TrYbe 18B.

The binding response for the combined hIL-17A/hTNF/HSA solution was equivalent to the sum of the responses of the independent injections, as shown in Table 7. This confirms that TrYbe 18B is capable of simultaneous binding to Human IL-17A, Human TNF and HSA.

TABLE 7

| Sample | Analyte | Binding (RU) |
|---|---|---|
| TrYbe18B | hTNF | 47 |
| | hIL- 17A | 38 |
| | HSA | 58 |
| | hTNF + hIL- | 130 |
| | 17A + HSA | (143) |

A further assay was performed to measure the simultaneous binding of Cynomolgus IL-17A, TNF and Albumin to TrYbe 18B. The TrYbe 18B construct was captured to the sensor chip by immobilised anti-human IgG-F(ab′)₂ then Cynomolgus IL-17A only, Cynomolgus TNF only, Cynomolgus HSA only or a mixed solution of Cynomolgus IL-17A, TNF and Albumin were titrated separately over the captured TrYbe 18B.

BIA (Biamolecular Interaction Analysis) was performed using a Biacore T200 (GE Healthcare). The TrYbe 18B construct was captured to the sensor chip surface as stated in the method above for Biacore kinetics for TrYbe 18B binding Cynomolgus TNF, IL-17A, IL-17F. 100 nM CSA, 20 nM cynoIL-17A, 20 nM cynoTNF or a mixed solution with final concentration of 100 nM HSA, 20 nM cynoIL-17A, 20 nM cynoTNF were titrated separately over the captured TrYbe 18B.

The binding response for the combined cynoIL-17A/cynoTNF/CSA solution was equivalent to the sum of the responses of the independent injections, as shown in Table 8. This confirms that TrYbe 18B is capable of simultaneous binding to Cynomolgus IL-17A, Cynomolgus TNF and CSA.

TABLE 8

| Sample | Analyte | Binding (RU) |
|---|---|---|
| TrYbe18B | cTNF | 42 |
| | cIL-17A | 44 |
| | CSA | 41 |
| | cTNF + cIL- | 118 |
| | 17A + CSA | (127) |

### Example 6 In Vitro Cell Based Activity of Fab496.g3-(HC)dsHLscFv2109 (LC)dsHLscFv645 (Trybe 18B) Against TNF (Human and Cyno)

The antibody molecule comprising Fab496.g3-(HC)dsscFv(HL)2109 (SEQ ID NO:125) (having the linker SGGGGSGGGGS connecting the Fab HC to the dsscFv2109), and Fab496.g3-(LC) dsscFv(HL)645 (SEQ ID NO: 131) (having the linker SGGGGSGGGGS connecting the FabLC to the dsscFv645), which is also referred to herein as antibody molecule Trybe 18B as produced according to the method in Example 3 was tested in an in vitro cell assay for activity against human TNF-alpha. The L929 cell line is a murine fibrosarcoma cell line which is sensitive to the cytotoxic effects of TNF-α. TNF stimulates via the TNF receptors, which bind Human, Cynomolgus or Murine TNF-α, to induce apoptosis. Cells are co-treated with Actinomycin D which increases the susceptibility to killing by TNF-α. The viability of the L929 cells following treatment with Actinomycin-D/TNF-α and TrYbe 18B was determined by detecting ATP levels (which decrease with decreasing viability) via a luciferase reaction (CellTiter-Glo, Promega).

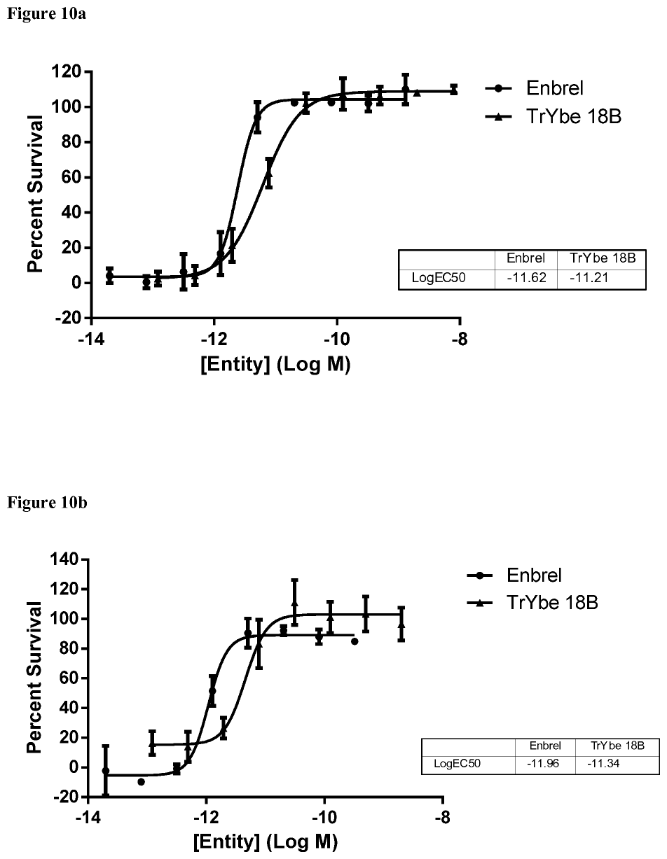

L929 cells were treated with 2.35 μg/ml Actinomycin D and human TNF alpha at 100 pg/ml in the presence of TrYbe 18B (concentration range 8.12E-9M to 1.23E-13M) or control compound Enbrel (concentration range 1.3E-9M-2E-14M) in a final volume of 30 μl in a 384 well flat bottom plate, following a 1 hour pre-incubation. After 24 hours at 37° C., 5% CO2 cell viability was measured by CellTiter-Glo (Promega Ltd). FIG. 10a shows representative cell survival curve for L929 cells treated with Actinomycin-D/and human TNF-α and TrYbe 18B or the control compound Enbrel.

L929 cells were also treated with 2.35 μg/ml Actinomycin D and cynomolgus TNF alpha at 20 pg/ml in the presence of TrYbe 18B (concentration range 8.12E⁻⁹M to 1.23E⁻¹³M) or control compound Enbrel (concentration range 1.3E⁻⁹M-2E⁻¹⁴M) in a final volume of 30 μl in a 384 well flat bottom plate, following a 1 hour pre-incubation. After 24 hours at 37° C., 5% CO₂ cell viability was measured by CellTiter-Glo (Promega Ltd). FIG. 10b shows representative cell survival curve for L929 cells treated with Actinomycin-D/and cynomolgus TNF-α and TrYbe 18B or the control compound Enbrel.

When stimulated by Human or Cynomolgus TNF-α, TrYbe 18B was able to fully inhibit the killing effect of TNF-α in a dose dependant manner; Mean EC₅₀ 5.87 pM (Human, N=3) and Mean EC₅₀ 4.97 pM (Cynomolgus, N=2). TrYbe 18B does not cross react with mouse TNF and therefore did not inhibit the killing effects of Murine TNF-α (N=3).

### Example 7 In Vitro Cell Based Activity of Fab496.g3-(HC)dsHLscFv2109 (LC)dsHLscFv645 (Trybe 18B) Against IL-17A and IL-17F

The antibody molecule comprising Fab496.g3-(HC)dsscFv(HL)2109 (SEQ ID NO:125) (having the linker SGGGGSGGGGS connecting the Fab HC to the dsscFv2109), and Fab496.g3-(LC) dsscFv(HL)645 (SEQ ID NO: 131) (having the linker SGGGGSGGGGS connecting the FabLC to the dsscFv645), which is also referred to herein as antibody molecule Trybe 18B as produced according to the method in Example 3 was tested in an in vitro cell assay for activity against human and cynomolgus IL-17A and F.

IL-17A and IL-17F in combination with IL-1β induce IL-6 release by the murine fibroblast cell line NIH 3T3. This forms the basis of an assay system in which to test the neutralising ability of anti-IL-17A and anti-IL-17F molecules.

TrYbe 18B or the anti-IL17A/F antibody CA028_00496.g3 described in WO2012/095662 (concentration range 5000 ng/mL to 19.5 ng/mL) were pre-incubated with recombinant human IL-17A (30 ng/mL) and recombinant human IL-1β (50 pg/mL) at 37° C. for 4 hr. The TrYbe/antibody protein complexes were then transferred to NIH-3T3 cells in 96-well flat-bottomed plates. After 3 days incubation at 37° C., 5% CO2, 100% humidity, cell-free supernatant was collected and the levels of IL-6 were determined by MSD.

TrYbe 18B or the anti-IL17A/F antibody CA028_00496.g3 (concentration range 5000 ng/mL to 19.5 ng/mL) were pre-incubated with recombinant human IL-17F (300 ng/mL) and recombinant human IL-1β (50 pg/mL) at 37° C. for 4 hr. The TrYbe/antibody protein complexes were then transferred to NIH-3T3 cells in 96-well flat-bottomed plates. After 3 days incubation at 37° C., 5% CO₂, 100% humidity, cell-free supernatant was collected and the levels of IL-6 were determined by MSD.

TrYbe 18B or the anti-IL17A/F antibody CA028_00496.g3 (concentration range 5000 ng/mL to 19.5 ng/mL) were pre-incubated with recombinant cyno IL-17A (30 ng/mL) and recombinant human IL-1β (50 pg/mL) at 37° C. for 4 hr. The TrYbe/antibody protein complexes were then transferred to NIH-3T3 cells in 96-well flat-bottomed plates. After 3 days incubation at 37° C., 5% CO₂, 100% humidity, cell-free supernatant was collected and the levels of IL-6 were determined by MSD.

TrYbe 18B or the anti-IL17A/F antibody CA028_00496.g3 (concentration range 5000 ng/mL to 19.5 ng/mL) were pre-incubated with recombinant cyno IL-17F (300 ng/mL) and IL-13 (50 pg/mL) at 37° C. for 4 hr. The tribody/antibody protein complexes were then transferred to NIH-3T3 cells in 96-well flat-bottomed plates. After 3 days incubation at 37° C., 5% CO₂, 100% humidity, cell-free supernatant was collected and the levels of IL-6 were determined by MSD.

TrYbe 18B or the anti-IL17A/F antibody CA028_00496.g3 (concentration range 5000 ng/mL to 19.5 ng/mL) were pre-incubated with recombinant cyno IL-17F (300 ng/mL) and IL-13 (50 pg/mL) at 37° C. for 4 hr. The tribody/antibody protein complexes were then transferred to NIH-3T3 cells in 96-well flat-bottomed plates. After 3 days incubation at 37° C., 5% CO₂, 100% humidity, cell-free supernatant was collected and the levels of IL-6 were determined by MSD.

In the NIH 3T3 bioassay TrYbe 18B inhibited both human and cynomolgus monkey (cyno) IL-17A and IL-17F induced IL-6 release in a concentration-dependent manner (data not shown). TrYbe 18B had equivalent potency to the control antibody CA028_00496.g3 (data not shown).

### Example 8 In Vivo Activity of Fab496.g3-(HC)dsHLscFv2109 (LC)dsHLscFv645 (Trybe 18B) Against TNF-Alpha

The antibody molecule comprising Fab496.g3-(HC)dsscFv(HL)2109 (SEQ ID NO:125) (having the linker SGGGGSGGGGS connecting the Fab HC to the dsscFv2109), and Fab496.g3-(LC) dsscFv(HL)645 (SEQ ID NO: 131) (having the linker SGGGGSGGGGS connecting the FabLC to the dsscFv645), which is also referred to herein as antibody molecule Trybe 18B as produced according to the method in Example 3 was tested in an in vivo assay to test inhibition of TNF-alpha.

The peritoneal administration of exogenous human TNF alpha in mice evokes a local inflammatory response associated with neutrophila. This response is largely elicited through the activation of mouse TNF receptor I (TNFRI) and the subsequent release of neutrophilic chemokines following NF-KB-mediated transcription. This model of human TNF alpha-induced neutrophilia can be used to determine the in vivo efficacy and potency of TrYbe 18B against human TNF alpha in a physiological system that is independent of human IL-17A/F. As human TNF alpha is the sole inflammatory stimulus in this model it was hypothesised that prophylactic treatment with TrYbe 18B would inhibit human TNF alpha-induced peritoneal neutrophilia.

Balb/c mice were treated intravenously with PBS or increasing concentrations of TrYbe 18B (0.1, 1 or 10 mg/kg). 1 hour post PBS or TrYbe 18B treatment, mice were challenged intraperitoneally with PBS or human TNF alpha (0.3 μg/kg). 4 hours later mice were humanely terminated and peritoneal lavage was collected for the assessment of neutrophilia by flow cytometry. Results are the mean (+/−SEM) of n=8/group. Statistical comparisons were calculated using one-way ANOVA with Dunnett's post-test (****=p<0.0001).

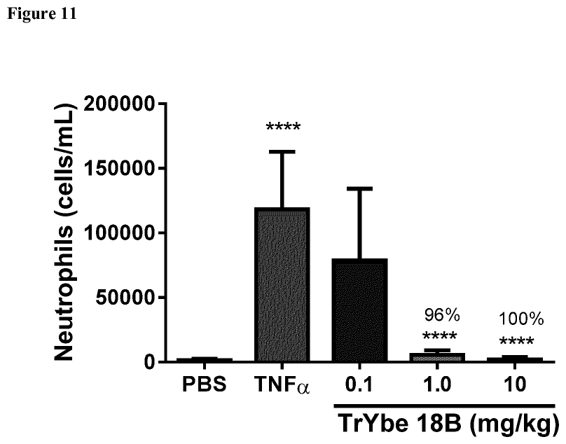

FIG. 11 shows that TrYbe 18B is able to dose-dependently inhibit human TNF alpha-induced neutrophila with almost complete ablation of the response at 1 and 10 mg/kg (96% and 100% inhibition respectively). As there is no human IL-17A/F in this system, this suggests that the anti-human TNF alpha dsscFv of TrYbe 18B is efficacious and potent in vivo.

### Example 9 In Vivo Activity of Fab496.g3-(HC)dsHLscFv2109 (LC)dsHLscFv645 (Trybe 18B) Against TNF-Alpha and IL-17A

The antibody molecule comprising Fab496.g3-(HC)dsscFv(HL)2109 (SEQ ID NO:125) (having the linker SGGGGSGGGGS connecting the Fab HC to the dsscFv2109), and Fab496.g3-(LC) dsscFv(HL)645 (SEQ ID NO: 131) (having the linker SGGGGSGGGGS connecting the FabLC to the dsscFv645), which is also referred to herein as antibody molecule Trybe 18B as produced according to the method in Example 3 was tested in an in vivo assay to test inhibition of TNF-alpha and IL-17A.

It is important to show that the TrYbe 18B antibody can inhibit a biological response that results from both cytokines being present at the same time. Previous studies have shown that the combination of human TNF alpha and IL-17A in vivo yields a synergistic neutrophilic response greater than the sum of either stimulus alone. The in vivo efficacy and potency of TrYbe 18B was therefore tested against human TNF alpha in combination with human IL-17A. To further discriminate between the synergistic contributions of human TNF alpha and human IL-17A in this model, monospecific antibodies against human TNF alpha (101.4) and human IL-17A/F (antibody CA028_00496.g3) were used.

Balb/c mice were treated intravenously with PBS, antibody CA028_00496.g3 (30 mg/kg), 101.4 (30 mg/kg) or increasing concentrations of TrYbe 18B (0.1, 1 or 10 mg/kg). 1 hour post antibody administration, mice were challenged intraperitoneally with PBS, human IL-17A (10 μg/kg), human TNF alpha (0.3 μg/kg) or a combination of human IL-17A (10 μg/kg) with human TNF alpha (0.3 μg/kg). 4 hours later mice were humanely terminated and peritoneal lavage was collected for the assessment of neutrophilia by flow cytometry. Results are the mean (+/−SEM) of n=8/group. Statistical comparisons were calculated using one-way ANOVA with Dunnett's post-test (*=p<0.05, **=p<0.01=***=p<0.001=****=p<0.0001).

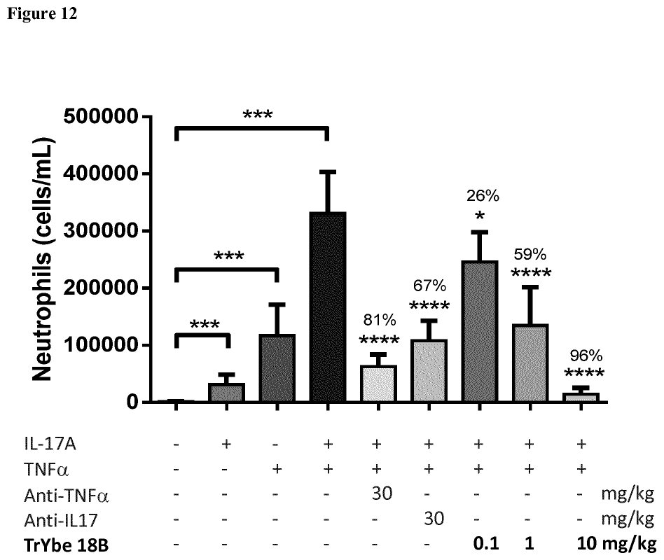

FIG. 12 shows that the intraperitoneal administration of human IL-17A in combination with human TNF alpha induces a local neutrophilic response much greater than the sum of either stimulus administered alone. Furthermore the inhibition of human TNF alpha in this model (using an excess of anti-TNFalpha antibody 101.4) reduced the combined IL-17A/TNF alpha-induced response to a level similar to that observed following IL-17A administration alone. Similarly, inhibition of human IL-17A/F (using an excess of antibody CA028_00496.g3) could only inhibit the combined IL-17A/TNF alpha-induced response to the same level observed following TNF alpha administration alone. In contrast, TrYbe 18B was able to dose-dependently inhibit IL-17A/TNF alpha-induced neutrophilia to a level beyond that achieved by either antibody CA028_00496.g3 or 101.4 (96% inhibition at 10 mg/kg TrYbe 18B vs. 81% and 67% inhibition with 30 mg/kg 101.4 and antibody CA028_00496.g3 respectively). Together this data shows that TrYbe 18B is able to simultaneously inhibit both human TNF alpha and human IL-17A in an in vivo model.

### Example 10 Biophysical Characterisation of Fab496.g3-(HC)dsHLscFv2109 (LC)dsHLscFv645 (Trybe 18B)

The antibody molecule comprising Fab496.g3-(HC)dsscFv(HL)2109 (SEQ ID NO:125) (having the linker SGGGGSGGGGS connecting the Fab HC to the dsscFv2109), and Fab496.g3-(LC) dsscFv(HL)645 (SEQ ID NO: 131) (having the linker SGGGGSGGGGS connecting the FabLC to the dsscFv645), which is also referred to herein as antibody molecule Trybe 18B, as produced according to the method in Example 3 and the antibody molecule comprising Fab496.g3-(HC)dsscFv(HL)2109 (SEQ ID NO:127) (having the linker SGGGGTGGGGS connecting the Fab HC and the dsscFv2109) and Fab496.g3-(LC) dsscFv(HL)645 (SEQ ID NO: 131) (having the linker SGGGGSGGGGS connecting the Fab LC and the dsscFv645), which is also referred to herein as antibody molecule Trybe 18T, as produced according to the method in Example 3 were tested in biophysical characterisation experiments.

Three batches of antibody molecules were analysed (see Table 9). This study resulted in determination of overall biochemical and biophysical characteristics of the TrYbe 18B molecule.

Additionally, a fraction (A3) from the purification of Stable batch 2 eluting differently from the parent TrYbe 18B molecule was analysed to confirm the identity and hence be able to explain unexpected impurities and/or biophysical characteristics of the TrYbe 18B molecule.

TABLE 9
Batches of TrYbe 18B used for biophysical characterisation

| | | Concentration |
|---|---|---|
| | Sample | (mg/ml) |
| Transient | TrYbe T (transient) | 4.6 |
| batch | | |
| Stable | TrYbe 18B (Mini | 2.64 |
| batch 1 | pool LP10) | |
| Stable | TrYbe 18B (Mini | 14.18 |
| batch 2 | pool MP77) | |

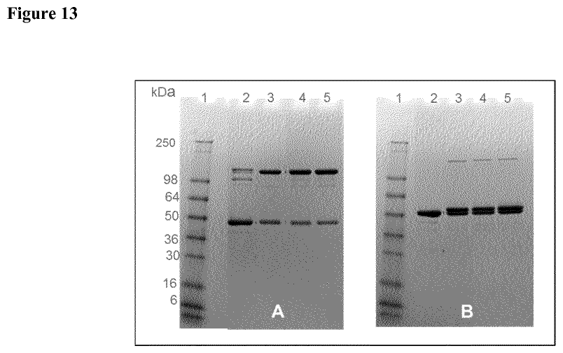

The purity of the batches was determined by Size Exclusion HPLC (SEC HPLC) (data not shown) and SDS PAGE (under non-reducing and reducing conditions) FIG. 13.

As judged by SEC HPLC, all samples eluted at the same retention time, with less than 2.2% high molecular weight species (Transient batch: 0.56%; Stable batch 1: 0.59%; Stable batch 2: 2.2%). The transient sample contained a low molecular weight contaminant as indicated by the broad peak eluting later than the main peak.

FIG. 13 shows SDS PAGE Analysis of Different Batches of TrYbe 18B using SDS PAGE 4-20% Novex Tris/Glycine (Invitrogen). Analysis under (A) non reducing (+N-ethylmaleimide (NEM) and (B) reducing conditions. Lane 1=See Blue Invitrogen Markers; Lane 2=A3 (fraction from purification of Stable batch 2); Lane 3=Transient batch; Lane 4=Stable batch 1; Lane 5=Stable batch 2. All three batches showed similar levels of purity by SDS PAGE with a band at ˜130 kDa (under non-reducing conditions) attributed to the fully assembled, disulphide paired antibody molecule and a doublet at ˜50 kDa (under reducing conditions) consistent with the respective reduced heavy and light chains. Under non reducing conditions, there was a band at ˜50 kDa which was equivalent to that observed with A3 (Lane 2). A3 was confirmed to be light chain capped with cysteine by intact mass spectrum analysis (50.8 kDa) and N terminal sequencing (predominant light chain). The band at 50 kDa (non-reducing gel) for the samples of interest was thought to be light chain since mass spectrum analysis did not discern any heavy chain. This was later confirmed by N terminal sequencing analysis of the bands, excised from a Ponseau S stained blot; only light chain N terminus (AIQLTQSPSS) was discerned.

pI Measurement:

Method 1: 30 μl protein sample at 1 mg/ml, 0.35% methylcellulose, 4% pH3-10 ampholytes (Pharmalyte), 1 μl of each synthetic pI marker (4.65 and 9.77) and HPLC grade water to make up the final volume to 100 μl. The mixture was then analysed using iCE3 IEF analyser (ProteinSimple), pre-focusing at 1500 V for 1 minute followed by focusing at 3000 V for 5 minutes.

The calibrated electropherograms were then integrated using Empower software (from Waters).

Method 2: 30 μl protein sample at 2 mg/ml, 105 ul of 1% methylcellulose, 12 ul pH3-8 ampholytes (Pharmalyte), 1.5 μl of each synthetic pI marker (4.65 and 9.77) and HPLC grade water to make up the final volume to 300 μl. The mixture was then analysed using iCE3 as for Method 1.

The experimental pI for all batches of TrYbe T and 18B was high (range 9.2-9.4) and hence is unlikely to have approximately zero overall molecular charge (when there is increased risk of aggregation) at the expected formulation pH (˜pH 5).

The pI of Fraction A3 was found to be pre-dominantly 8.31 (48.6%).

Molecular Stability:

The molecular stability was measured by melting temperature (Tm) (measure of unfolding) and effect of agitation (unfolding by physical stress followed by aggregation).

Tm analysis by Differential Scanning Calorimetry (DSC) of all batches of TrYbe T and 18B could distinguish two main domains. The lower unfolding event was attributed to the 2109 scFv. The buffer species and the pH did not appear to affect the melting temperatures.

The thermofluor assay resulted in Tm values (where observed) similar to those derived from the DSC analysis although it was not as easy to discriminate between the different domains. Only the higher unfolding domain was evident for Stable batch 1 and Stable batch 2. It was possible to discern earlier unfolding transitions (48-50° C.) for the transient batch, which could possibly be attributed to the presence of excess light chain.

TABLE 10
Derivation of Tm by Differential Scanning Calorimetry (DSC) and
Thermofluor assays: all TrYbe 18B molecules showed a lower initial unfolding event than an IgG4
molecule (~68° C.).

| | | | | Stable |
|---|---|---|---|---|
| | | Transient Batch | Stable batch 1 | batch 2 |
| Thermofluor | Analysis 1 (PBS | 3 transitions evident: | 61.63 ± 0.29° C.; | ND |
| | pH 7.40) | 48.85 ± 0.52° C.; 60.95 ± 0.35° C.; | 71.2 ± 0.23° C. | |
| | | 70.9 ± 0.2° C. | | |
| | Analysis 2 (PBS | 50.1 ± 0.6° C.; 71.3 ± 0.3° C. | 71.0 ± 0.2° C. | 71.9 ± 0.2 |
| | pH 7.40) | | | |
| | Analysis 2 (PBS | 71.0 ± 0.2; 61.4 ± 0.3° C. | 61.4 ± 0.3° C. | 71.5 ± 0.2° C. |
| | pH 7.40 + NEM) | | | |
| DSC | PBS pH 7.4 | 2 transitions: 63.8 ± 0.4° C.; | 2 transitions: | ND |
| | | 72.6 ± 0.1° C. | 62.9 ± 0° C.; | |
| | | | 71.8 ± 0.1° C. | |
| | Ac pH 5 | 2 transitions: 61.8 ± 0.1° C.; | 2 transitions: | ND |
| | | 72.2 ± 0.1° C. | 61.7 ± 0° C.; | |
| | | | 71.9 ± 0.1° C. | |

ND denotes that the analyses was not done (due to availability of material)

In summary, it was evident from the above analyses that there was no significant difference between the three batches of TrYbe T and 18B.

TrYbe 18B had a high pI, but exhibited a moderately low Tm compared to conventional formats (IgG and Fab′ molecules) which appeared to be governed by the CDR of the 2109scFv component of the molecule. All batches contained excess light chain which probably contributed to heterogeneity/instability that was observed, however, this could be removed by purification.

### Example 11 In Vitro Cell Based Activity of Fab496.g3-(HC)dsHLscFv2109 (LC)dsHLscFv645 (Trybe 18B) Against IL-17A and IL-17F

TNF alpha in addition to IL-17A and IL-17F isoforms are key cytokines produced by Th17 cells and known indirectly to induce the recruitment of neutrophils through the activation of non-haematopioetic tissue, such as synoviocytes of the joint. In this study, the efficacy of TrYbe18B in a complex in vitro model of neutrophil migration was compared against combined individual antibodies targeting IL-17AF and Enbrel targeting TNF alpha.

Activation of RA synoviocytes: Day 1: Cultured RA synoviocytes (passage 6) were seeded at 10⁴ cells per well in the lower chamber of a 24 well transwell plate and incubated for 24h at 37° C. Day 2: The following day, synoviocytes were activated with supernatant (at a 1:10 dilution) derived from Th17 cells (EWBE-037388) pre-blocked (1h room temperature) with IL-17A (antibody 497 as described in WO2008/001063) or IL-17F or IL-17AF (antibody CA028_00496.g3) specific antibodies with or without additional anti-TNF alpha neutralizing Enbrel. Th17 was also pre-incubated with the negative control A33 IgG or TrYbe18B to neutralize IL-17A+F and TNF alpha. Cultures were performed in a total volume of 0.5 ml of either control or stimulation media. All antibodies tested were used at in excess (10 ug/ml), to fully neutralize target cytokine present in Th17 supernatant. Stimulated fibroblasts were then incubated for an additional 24h prior to migration assay.

Neutrophil migration assay: Day 3: To isolate human White blood cells (leucocytes) from human whole blood, 40 ml ACK lysis buffer was mixed with 10 ml of human blood for 10 mins at room temperature to efficiently lysed RBC. Leucocytes were then spun at 400 g for 10 mins and further washed twice in 20 ml PBS prior to being counted at resuspended in plain RPMI Media (Gibco) at 10⁶ cells/ml. 5×10⁵ leucocytes (0.5 ml) were seeded onto the upper chamber of the transwell and incubated for 6h at 37° C. Leucocytes that migrated through the 3.0 uM permeable membrane into the lower chamber were isolated and labeled with anti-human CD18 specific antibody (Ebioscience) for 30 mins (2 ul/test on ice) to identify neutrophils. Antibody stained samples were then washed once in phosphate-buffered saline (PBS) pH7.4 prior to being prior to FACS acquisition on the BD LSRII Fortessa X20 cytometry analyser. All samples were resuspended in 200 μL of PBS spiked with Sigma reference beads. Statistical analysis was performed using a One-way Anova with Dunnet post test using IgG (-anti-TNF alpha) as comparator.

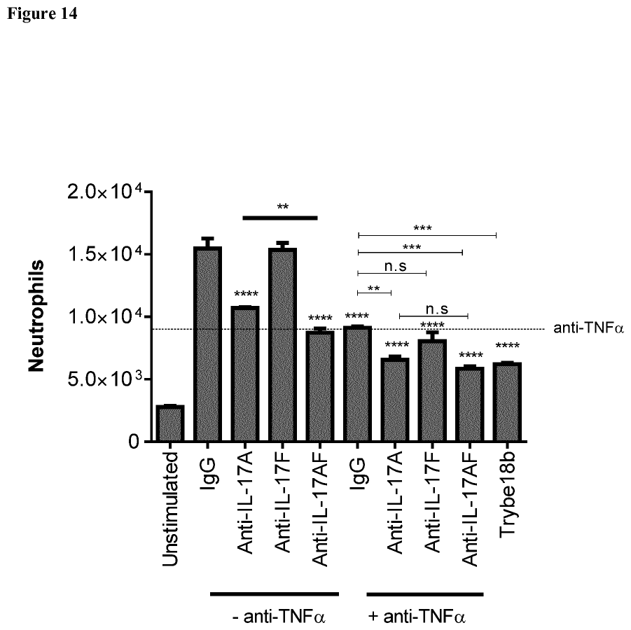

Using either TrYbe18B or IL-17 isoform specific and TNF alpha blocking antibodies it was possible to determine the individual and collective influence of IL-17 and TNF alpha in regulating migration of human neutrophils in a complex pre-clinical in vitro assay. RA synoviocytes activated using supernatants from Th17 cells significantly increased the chemotactic response of neutrophils (FIG. 14). Neutrophil migration was significantly suppressed by specific IL-17A blockade relative to isotype matched negative controls. Neutralization of IL-17F only in Th17 supernatant prior to RA synoviocyte activation did not alter neutrophil migratory capacity. In contrast, dual inhibition of IL-17A and IL-17F suppressed neutrophil migration to a greater degree than IL-17A inhibition only. Pre-blockade of TNF alpha using Enbrel also had a notable impact on neutrophil chemotaxis which was further suppressed by additional IL-17 isoform specific neutralizing antibodies. Importantly, tri-specific blockade of IL-17A, IL-17F and TNF using TrYbe18B demonstrated similar inhibition of neutrophil migration as using separate antibodies to neutralize these cytokines.

In this study, we demonstrated that optimal inhibition of neutrophil migration is achieved through neutralisation of IL-17AF and TNF alpha, either utilising separate blocking (Anti-IL-17AF+Enbrel) or through tri-specific blockade using TrYbe18B.

### Example 12 Pharmacokinetic (PK) Analysis of TrYbe 18B In Vivo

Four male cynomolgus monkeys were administered a 10 mg/kg intravenous (IV) bolus dose of TrYbe18B and serum samples collected at selected intervals for 28 days. Samples were analysed for concentrations of TrYbe18B using an immunoassay that confirms the presence of both the IL17A/F and TNF binding regions. Two compartmental PK analysis was conducted.

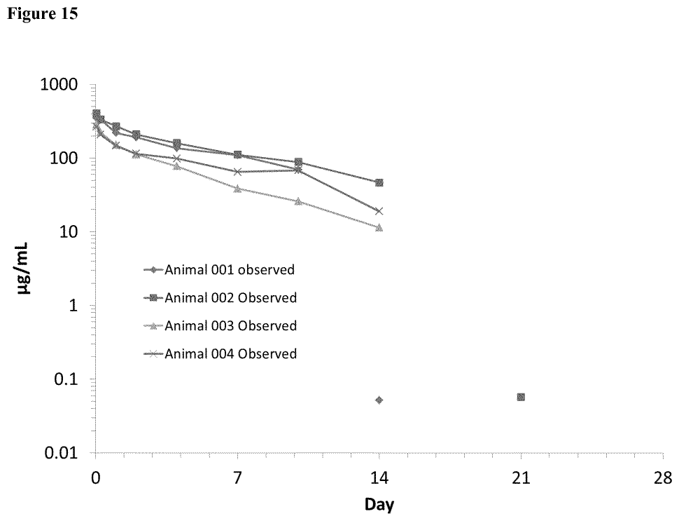

Serum concentrations of TrYbe18B were consistent with a large molecular weight protein with the capacity to bind to, and be recycled by, FcRn (FIG. 15). This property is conferred to TrYbe18B by its albumin binding domain. Initial concentrations after IV dosing of TrYbe18B were consistent with a molecule with a central compartment volume similar to plasma volume. Concentrations declined in a biexponential manner with a terminal (beta) half-life of approximately 5.6 days (Table 11). The slow terminal half-life is concordant with the relatively slow clearance of TrYbe18B; in alignment with the binding to, and recycling by, FcRn. The slow clearance also demonstrates stability of the TrYbe in vivo, as substantial cleavage of any of the binding components of the molecule would manifest as increased apparent clearance.

TABLE 11
Estimated PK parameters of TrYbe18B following an IV
dose to cynomolgus monkeys.

| | | Central | Steady | | |
|---|---|---|---|---|---|
| | | compartment | state | | Terminal |
| | Cₘₐₓ | volume | volume | CL | half-life |
| Animal | (μg/mL) | (mL/kg) | (mL/kg) | (mL/day/kg) | (days) |
| Mean | 329 | 31.2 | 48.1 | 6.70 | 5.59 |
| SD | 62.8 | 6.01 | 12.8 | 3.01 | 1.29 |

CL = Clearance
Cmax = maximum serum concentration
SD = standard deviation

1. A multi-specific antibody molecule comprising a binding domain specific to human TNF-alpha and a binding domain specific to human IL-17A and human IL-17F, wherein the antibody molecule is capable of neutralising the biological activity of human TNF-alpha, human IL-17A and human IL-17F.

2. A multi-specific antibody molecule according to claim 1, wherein the antibody has a binding affinity for human TNF-alpha of 200 pM or better.

3. A multi-specific antibody molecule according to claim 1 or claim 2, wherein the antibody has a binding affinity for human IL-17A of 100 pM or better.

4. A multi-specific antibody molecule according to claim 3, wherein the antibody has a binding affinity for human IL-17A of 20 pM or better.

5. A multi-specific antibody molecule according to any one of claims 1 to 4, wherein the antibody has a binding affinity for IL-17F of 100 pM or better.

6. A multi-specific antibody molecule according to claim 5, wherein the antibody has a binding affinity for IL-17 F of 20 pM or better.

7. A multi-specific antibody molecule according to any one of claims 1 to 6, wherein the binding domain specific to human IL-17A and human IL-17F also binds the IL-17A/IL-17F heterodimer.

8. A multispecific antibody molecule according to any one of claims 1 to 7, wherein each binding domain comprises two antibody variable domains.

9. A multispecific antibody molecule according to claim 8, wherein the two antibody variable domains are a VH/VL pair.

10. A multispecific antibody molecule according to any one of claims 1 to 9, wherein the molecule format is selected from diabody, scdiabody, triabody, tandem scFv, FabFv, Fab′Fv, FabdsFv, Fab-scFv, Fab-dsscFv, Fab-(dsscFv)₂ diFab, diFab′, tribody, tandem scFv-Fc, scFv-Fc-scFv, scdiabody-Fc, scdiabody-CH3, Ig-scFv, scFv-Ig, V-Ig, Ig-V, Duobody and DVD-Ig.

11. A multispecific antibody molecule according to any one of claims 1 to 9 wherein the binding domain which is specific to human TNF-alpha and the binding domain which is specific to IL-17A and human IL-17F are independently selected from a Fab, scFv, Fv, dsFv and dsscFv.

12. A multi-specific antibody molecule according to any one of claims 1 to 9 comprising or consisting of:
V_{H1}-CH₁-X-V₁; and c) a polypeptide chain of formula (I):
V_{L1}-C_{L}-Y-V₂; d) a polypeptide chain of formula (II):
wherein:
V_{H1} represents a heavy chain variable domain;
CH₁ represents a domain of a heavy chain constant region, for example domain 1 thereof;
X represents a bond or linker;
Y represents a bond or linker;
V₁ represents a dsFv, a sdAb, a scFv or a dsscFv;
V_{L1} represents a light chain variable domain;
C_{L} represents a domain from a light chain constant region, such as Ckappa;
V₂ represents dsFv, a sdAb, a scFv or a dsscFv.

13. A multi-specific antibody molecule according to claim 12, wherein at least one of V₁ and V₂ is a scFv or dsscFv.

14. A multi-specific antibody molecule according to claim 13, wherein V₁ is a scFv or dsscFv and V₂ is a scFv or dsscFv.

15. A multi-specific antibody molecule according to claim 14, wherein V1 is a dsscFv and V2 is a dsscFv.

16. A multi-specific antibody molecule according to any one of claims 12 to 15, wherein V1 is a scFv or dsscFv, and V1 comprises a polypeptide chain of:
a. formula (III): VH2-Z1-VL2 and VH2 is attached to X, for example through a peptide bond; or
b. formula (IV): VL2-Z1-VH2 and VL2 is attached to X, for example through a peptide bond;
wherein VH2 represents a heavy chain variable domain, Z1 represents a peptide linker and VL2 represents a light chain variable domain.

17. A multispecific antibody molecule according to any one of claims 12 to 16, wherein V2 is a scFv or dsscFv, and V2 comprises a polypeptide chain of:
a. formula (V): VH3-Z2-VL3 and VH3 is attached to Y, for example through a peptide bond; or
b. formula (VI): VL3-Z2-VH3 and VL3 is attached to Y, for example through a peptide bond;
wherein VH3 represents a heavy chain variable domain, Z2 represents a peptide linker and VL3 represents a light chain variable domain.

18. A multispecific antibody according to claim 16 or 17, wherein Z1 and/or Z2 are a peptide linker of 12 to 25 amino acids in length, for example the sequence given in SEQ ID NO: 68.

19. A multi-specific antibody molecule according to any one of claims 12 to 18, wherein X is a peptide linker, for example the sequence given in SEQ ID NO: 1 or 2.

20. A multi-specific antibody molecule according to any one of claims 12 to 19, wherein Y is a peptide linker, for example the sequence given in SEQ ID NO: 1 or 2.

21. A multi-specific antibody molecule according to claim 19 or claim 20, wherein X is the sequence given in SEQ ID NO: 2 and Y is the sequence given in SEQ ID NO:2.

22. A multi-specific antibody molecule according to claim 19 or claim 20, wherein X is the sequence given in SEQ ID NO: 1 and Y is the sequence given in SEQ ID NO:2.

23. A multi-specific antibody molecule according to any one of claims 12 to 22 wherein at least one of V1 and V2 is a dsscFv or dsFv; and the light chain and heavy chain variable domains of V₁ and/or the light chain and heavy chain variable domains of V₂ are linked by a disulfide bond between two engineered cysteine residues.

24. A multi-specific antibody molecule according to any one of claims 12-23, wherein at least one of V1 and V2 is a dsscFv or dsFv; and the heavy chain and light chain variable domains of V₁ and/or V₂ are linked by a disulfide bond between two cysteine residues, wherein the position of the pair of cysteine residues is selected from the group comprising or consisting of: V_{H}37 and V_{L}95, V_{H}44 and V_{L}100, V_{H}44 and V_{L}105, V_{H}45 and V_{L}87, V_{H}100 and V_{L}50, V_{H}100b and V_{L}49, V_{H}98 and V_{L}46, V_{H}101 and V_{L}46, V_{H}105 and V_{L}43 and V_{H}106 and V_{L}57, wherein the V_{H} and V_{L} values are independently selected within a given V₁ or V₂.

25. A multi-specific antibody molecule according to claim 24, wherein the position of the pair of engineered cysteine residues is V_{H}44 and V_{L}100.

26. A multi-specific antibody molecule according to any one of claims 12 to 25, wherein VH1 and VL1 comprise the binding domain specific to human IL-17A and human IL-17F and V1 and/or V2 comprise the binding domain specific to human TNF-alpha.

27. A multi-specific antibody molecule according to any one of claims 1 to 26 which comprises three binding domains.

28. A multi-specific antibody molecule according to claim 27 wherein each of the three binding domains bind different antigens.

29. A multi-specific antibody molecule according to claim 28, wherein the antibody molecule comprises a binding domain specific to a human serum carrier protein.

30. A multi-specific antibody molecule according to claim 29, wherein the human serum carrier protein is selected from the group consisting of thyroxine-binding protein, transthyretin, α1-acid glycoprotein, transferrin, fibrinogen and albumin, or a fragment of any thereof.

31. A multi-specific antibody molecule according to any one of claims 12 to 30, wherein:
a. VH1 and VL1 comprise the binding domain specific to human IL-17A and human IL-17F; and
b. V1 comprises the binding domain specific to human TNF-alpha and V2 comprises a binding domain specific to a human serum carrier protein, for example human serum albumin; or V2 comprises the binding domain specific to human TNF-alpha and V₁ comprises a binding domain specific to a human serum carrier protein, for example human serum albumin.

32. A multispecific antibody molecule according to any one of claims 1 to 31 wherein the antibody molecule comprises no more than one binding domain which is specific to human TNF-alpha and no more than one binding domain which is specific to human IL-17A and human IL-17F.

33. A multispecific antibody molecule according to any one of claims 1 to 32, wherein the antibody molecule does not comprise a CH2 domain and/or a CH3 domain.

34. A multi-specific antibody molecule according to any one of claims 1 to 33, wherein the binding domain specific for human TNF-alpha comprises at least one of a CDR having the sequence given in SEQ ID NO:85 for CDRH1, a CDR having the sequence given in SEQ ID NO:86 for CDRH2 and a CDR having the sequence given in SEQ ID NO:87 for CDRH3.

35. A multi-specific antibody molecule according to claim 34, wherein the binding domain specific for human TNF-alpha comprises 3 heavy chain CDRs having the sequence given in SEQ ID NO:85 for CDRH1, SEQ ID NO:86 for CDRH2 and SEQ ID NO:87 for CDRH3.

36. A multi-specific antibody molecule according to any one of claims 1 to 35, wherein the binding domain specific for human TNF-alpha comprises at least one of a CDR having the sequence given in SEQ ID NO: 88 for CDRL1, a CDR having the sequence given in SEQ ID NO:89 for CDRL2 and a CDR having the sequence given in SEQ ID NO:90 for CDRL3.

37. A multi-specific antibody molecule according to claim 36, wherein the binding domain specific for human TNF-alpha comprises 3 light chain CDRs having the sequence given in SEQ ID NO:88 for CDRL1, SEQ ID NO:89 for CDRL2 and SEQ ID NO:90 for CDRL3.

38. A multi-specific antibody molecule according to any one of claims 1 to 37, wherein the binding domain specific for human TNF-alpha comprises a heavy chain variable domain comprising the sequence given in SEQ ID NO:92 or SEQ ID NO:96; a sequence having at least 80% identity or similarity to the sequence given in SEQ ID NO:92 or SEQ ID NO:96; or a sequence having at least 80% identity or similarity to the sequence given in SEQ ID NO: 92 or SEQ ID NO:96 wherein CDRH1, CDRH2 and CDRH2 have the sequences given in claim 35.

39. A multispecific antibody molecule according to any one of claims 1 to 38, wherein the binding domain specific for human TNF-alpha comprises a light chain variable domain comprising the sequence given in SEQ ID NO:91 or SEQ ID NO:95; a sequence having at least 80% identity or similarity to the sequence given in SEQ ID NO:91 or SEQ ID NO:95; or a sequence having at least 80% identity or similarity to the sequence given in SEQ ID NO:91 or SEQ ID NO:95 wherein CDRL1, CDRL2 and CDRL3 have the sequences given in claim 37.

40. A multispecific antibody according to any one of claims 1 to 39, wherein the antibody comprises a dsscFv specific for human TNF-alpha and the dsscFv comprises the sequence given in SEQ ID NO: 101; a sequence having at least 80% identity or similarity to the sequence given in SEQ ID NO: 101; or a sequence having at least 80% identity or similarity to the sequence given in SEQ ID NO:101 wherein CDRH1, CDRH2 and CDRH3 have the sequences given in claim 35 and CDRL1, CDRL2 and CDRL3 have the sequences given in claim 37.

41. A multispecific antibody according to any one of claims 1 to 39, wherein the antibody comprises a scFv specific for human TNF-alpha and the scFv comprises the sequence given in SEQ ID NO:99; a sequence having at least 80% identity or similarity to the sequence given in SEQ ID NO:99; or a sequence having at least 80% identity or similarity to the sequence given in SEQ ID NO:99 wherein CDRH1, CDRH2 and CDRH3 have the sequences given in claim 35 and CDRL1, CDRL2 and CDRL3 have the sequences given in claim 37.

42. A multi-specific antibody molecule according to any one of claims 1 to 41, wherein the binding domain specific for human IL-17A and human IL-17F comprises 3 heavy chain CDRs having the sequence given in SEQ ID NO: 71 for CDRH1, SEQ ID NO: 72 for CDRH2 and SEQ ID NO: 73 for CDRH3.

43. A multi-specific antibody molecule according to any one of claims 1 to 42, wherein the binding domain specific for human IL-17A and human IL-17F comprises 3 light chain CDRs having the sequence given in SEQ ID NO:74 for CDRL1, SEQ ID NO:75 for CDRL2 and SEQ ID NO:76 for CDRL3.

44. A multi-specific antibody molecule according to any one of claims 1 to 43, wherein the binding domain specific for human IL-17A and human IL-17F comprises a heavy chain variable domain comprising the sequence given in SEQ ID NO:78; a sequence having at least 80% identity or similarity to the sequence given in SEQ ID NO:78; or a sequence having at least 80% identity or similarity to the sequence given in SEQ ID NO:78 wherein CDRH1, CDRH2 and CDRH3 have the sequences given in claim 42.

45. A multi-specific antibody molecule according to any one of claims 1 to 44, wherein the binding domain specific for human IL-17A and human IL-17F comprises a light chain variable domain comprising the sequence given in SEQ ID NO:77; a sequence having at least 80% identity or similarity to the sequence given in SEQ ID NO:77; or a sequence having at least 80% identity or similarity to the sequence given in SEQ ID NO:77 wherein CDRL1, CDRL2 and CDRL3 have the sequences given in claim 43.

46. A multi-specific antibody molecule according to any one of claims 1 to 45, wherein the antibody comprises a heavy chain comprising the sequence given in SEQ ID NO:82; a sequence having at least 80% identity or similarity to the sequence given in SEQ ID NO:82; or a sequence having at least 80% identity or similarity to the sequence given in SEQ ID NO:82 wherein CDRH1, CDRH2 and CDRH3 have the sequences given in claim 42.

47. A multi-specific antibody molecule according to any one of claims 1 to 46, wherein the antibody comprises a light chain comprising the sequence given in SEQ ID NO:81; a sequence having at least 80% identity or similarity to the sequence given in SEQ ID NO:81; or a sequence having at least 80% identity or similarity to the sequence given in SEQ ID NO:81 wherein CDRL1, CDRL2 and CDRL3 have the sequences given in claim 43.

48. A multi-specific antibody molecule according to any one of claims 1 to 47, wherein the antibody molecule comprises a binding domain specific for human serum albumin which comprises 3 heavy chain CDRs having the sequence given in SEQ ID NO: 103 for CDRH1, SEQ ID NO: 104 for CDRH2 and SEQ ID NO: 105 for CDRH3.

49. A multi-specific antibody molecule according to any one of claims 1 to 48, wherein the antibody molecule comprises a binding domain specific for human serum albumin which comprises 3 light chain CDRs having the sequence given in SEQ ID NO:106 for CDRL1, SEQ ID NO: 107 for CDRL2 and SEQ ID NO: 108 for CDRL3.

50. A multi-specific antibody molecule according to any one of claims 1 to 49, wherein the binding domain specific for human serum albumin comprises a heavy chain variable domain comprising the sequence given in SEQ ID NO:110 or SEQ ID NO:114; a sequence having at least 80% identity or similarity to the sequence given in SEQ ID NO:110 or SEQ ID NO:114; or having at least 80% identity or similarity to the sequence given in SEQ ID NO:110 or SEQ ID NO: 114 wherein CDRH1, CDRH2 and CDRH3 have the sequences given in claim 48.

51. A multispecific antibody molecule according to any one of claims 1 to 50, wherein the binding domain specific for human serum albumin comprises a light chain variable domain comprising the sequence given in SEQ ID NO: 109 or SEQ ID NO: 113 a sequence having at least 80% identity or similarity to the sequence given in SEQ ID NO: 109 or SEQ ID NO: 113; or a sequence having at least 80% identity or similarity to the sequence given in SEQ ID NO: 109 or SEQ ID NO: 113 wherein CDRL1, CDRL2 and CDRL3 have sequences given in claim 49.

52. A multispecific antibody according to any one of claims 1 to 51, wherein the antibody comprises a dsscFv specific for human serum albumin and the dsscFv comprises the sequence given in SEQ ID NO: 119; a sequence having at least 80% identity or similarity to the sequence given in SEQ ID NO: 119; or a sequence having at least 80% identity or similarity to the sequence of SEQ ID NO:119 wherein CDRH1, CDRH2 and CDRH3 have the sequences given in claim 48 and CDRL1, CDRL2 and CDRL3 have the sequences given in claim 49.

53. A multispecific antibody according to any one of claims 1 to 51, wherein the antibody comprises a scFv specific for human serum albumin and the scFv comprises the sequence given in SEQ ID NO: 117; a sequence having at least 80% identity or similarity to the sequence given in SEQ ID NO: 117; or a sequence having at least 80% identity or similarity to the sequence given in SEQ ID NO: 117 wherein CDRH1, CDRH2 and CDRH3 have the sequences given in claim 48 and CDRL1, CDRL2 and CDRL3 have the sequences given in claim 49.

54. A tri-specific antibody molecule comprising a binding domain specific to human TNF-alpha, a binding domain specific to human IL-17A and human IL-17F and a binding domain specific to human serum albumin, wherein the antibody molecule comprises or consists of:
a. a first polypeptide comprising or consisting of the sequence given in SEQ ID NO: 125; a sequence having at least 80% identity or similarity to the sequence given in SEQ ID NO: 125; or a sequence having at least 80% identity or similarity to the sequence given in SEQ ID NO: 125 wherein the CDRs have the sequences given in claims 35, 37 and 42; and
b. a second polypeptide comprising or consisting of the sequence given in SEQ ID NO: 131; a sequence having at least 80% identity or similarity to the sequence given in SEQ ID NO: 131; or a sequence having at least 80% identity or similarity to the sequence given in SEQ ID NO: 131 wherein the CDRs have the sequences given in claims 43, 48 and 49.

55. A tri-specific antibody molecule comprising a binding domain specific to human TNF-alpha, a binding domain specific to human IL-17A and human IL-17F and a binding domain specific to human serum albumin, and comprises or consists of:
a. a first polypeptide comprising or consisting of the sequence given in SEQ ID NO: 127; a sequence having at least 80% identity or similarity to the sequence given in SEQ ID NO: 127; or a sequence having at least 80% identity or similarity to the sequence given in SEQ ID NO:127 wherein the CDRs have the sequences given in claims 35, 37 and 42; and
b. a second polypeptide comprising or consisting of the sequence given in SEQ ID NO:131; a sequence having at least 80% identity or similarity to the sequence given in SEQ ID NO:131; or a sequence having at least 80% identity or similarity to the sequence given in SEQ ID NO: 131 wherein the CDRs have the sequences given in claims 43, 48 and 49.

56. A tri-specific antibody molecule according to claim 54 or claim 55, wherein the antibody molecule is capable of neutralising the biological activity of human TNF-alpha, human IL-17A and human IL-17F.

57. A polynucleotide encoding a multi-specific antibody molecule or polypeptide chain thereof according to any one of claims 1 to 56.

58. A vector comprising a polynucleotide or polynucleotides defined in claim 57.

59. A host cell comprising one or more polynucleotides or vectors of claim 57 or 58 respectively.

60. A host cell comprising two vectors each vector comprising a polynucleotide encoding a different polypeptide chain of a multi-specific antibody molecule according to any one of claims 1 to 56.

61. A process comprising expressing a multi-specific antibody molecule from a host cell defined in claim 59 or claim 60.

62. A pharmaceutical composition comprising a multi-specific antibody molecule according to any one of claims 1 to 56 and at least one excipient.

63. A multi-specific antibody molecule according to any one of claims 1 to 56 or a pharmaceutical composition according to claim 62 for use in therapy.

64. A method of treating a patient in need thereof comprising administering a therapeutically effective amount of a multi-specific antibody molecule according to any one of claims 1 to 56 or a pharmaceutical composition according to claim 62.

65. A tri-specific antibody molecule comprising a binding domain specific to human TNF-alpha, a binding domain specific to human IL-17A and human IL-17F and a binding domain specific to human serum albumin, and comprises or consists of a first polypeptide encoded by the polynucleotide sequence given in SEQ ID NO:126 and a second polypeptide encoded by the polynucleotide sequence given in SEQ ID NO: 132.
